SEQUENCE LISTING (90 sequences)

SEQ ID NO: 1 - DNA, 21 bp, Artificial Sequence
  Synthetic forward primer
ttcctggtccacaacgtcaag

SEQ ID NO: 2 - DNA, 18 bp, Artificial Sequence
  Synthetic reverse primer
tgtgagcgatctcggcac

SEQ ID NO: 3 - DNA, 14 bp, Artificial Sequence
  Synthetic forward primer
gcggcgcggaccat

SEQ ID NO: 4 - DNA, 21 bp, Artificial Sequence
  Synthetic reverse primer
cctggacaggaacagcagcta

SEQ ID NO: 5 - DNA, 22 bp, Artificial Sequence
  Synthetic forward primer
gggcagggcattattaggctat

SEQ ID NO: 6 - DNA, 25 bp, Artificial Sequence
  Synthetic reverse primer
ttaggttgtcagcctctatgttgaa

SEQ ID NO: 7 - DNA, 18 bp, Artificial Sequence
  Synthetic forward primer
ggcccagttgtgcgtctt

SEQ ID NO: 8 - DNA, 20 bp, Artificial Sequence
  Synthetic reverse primer
cgagccaggctttcacttct

SEQ ID NO: 9 - DNA, 21 bp, Artificial Sequence
  Synthetic forward primer
tggacctcagcttacccaaca

SEQ ID NO: 10 - DNA, 20 bp, Artificial Sequence
  Synthetic reverse primer
gactgaagcctggccacatc

SEQ ID NO: 11 - DNA, 18 bp, Artificial Sequence
  Synthetic forward primer
ccgcgtgtctcacccttt

SEQ ID NO: 12 - DNA, 19 bp, Artificial Sequence
  Synthetic reverse primer
gaccgtgccctcagctcat

SEQ ID NO: 13 - DNA, 19 bp, Artificial Sequence
  Synthetic forward primer
tgaactccgcgtgctcatc

SEQ ID NO: 14 - DNA, 21 bp, Artificial Sequence
  Synthetic reverse primer
cggtactgcctgtcagcttct

SEQ ID NO: 15 - DNA, 24 bp, Artificial Sequence
  Synthetic forward primer
tttccaggttgttttacgaatacg

SEQ ID NO: 16 - DNA, 19 bp, Artificial Sequence
  Synthetic reverse primer
tcctcaagctcggctggat

SEQ ID NO: 17 - DNA, 24 bp, Artificial Sequence
  Synthetic forward primer
cttcctatagctgcagccatgtac

SEQ ID NO: 18 - DNA, 19 bp, Artificial Sequence
  Synthetic reverse primer
gcattggcgtgctccttct

SEQ ID NO: 19 - DNA, 20 bp, Artificial Sequence
  Synthetic forward primer
tcagaccagtcgcagtttcg

SEQ ID NO: 20 - DNA, 17 bp, Artificial Sequence
  Synthetic reverse primer
ctgcgttgcgcatttcc

SEQ ID NO: 21 - DNA, 22 bp, Artificial Sequence
  Synthetic forward primer
cgtgctcctcttggtacctcat

SEQ ID NO: 22 - DNA, 20 bp, Artificial Sequence
  Synthetic reverse primer
cggtcaaggcggagattatc

SEQ ID NO: 23 - DNA, 22 bp, Artificial Sequence
  Synthetic forward primer
tgcagaaggctcatgagttcct

SEQ ID NO: 24 - DNA, 22 bp, Artificial Sequence
  Synthetic reverse primer
tctggtagtcgggagggttatc

SEQ ID NO: 25 - DNA, 19 bp, Artificial Sequence
  Synthetic forward primer
tgcagcctggctcttacca

SEQ ID NO: 26 - DNA, 20 bp, Artificial Sequence
  Synthetic reverse primer
agctctgtccctgcgtctga

SEQ ID NO: 27 - DNA, 17 bp, Artificial Sequence
  Synthetic forward primer
gccaccctcaccgcttt

SEQ ID NO: 28 - DNA, 21 bp, Artificial Sequence
  Synthetic reverse primer
gctacctgcgccttcatgtag

SEQ ID NO: 29 - DNA, 18 bp, Artificial Sequence
  Synthetic forward primer
gaaaagccggcaccaaga

SEQ ID NO: 30 - DNA, 25 bp, Artificial Sequence
  Synthetic reverse primer
tcaaagagagaatcagctcaaactg

SEQ ID NO: 31 - DNA, 21 bp, Artificial Sequence
  Synthetic forward primer
agaatcaggccaagagatgca

SEQ ID NO: 32 - DNA, 18 bp, Artificial Sequence
  Synthetic reverse primer
tgtgctgccccaggaatc

SEQ ID NO: 33 - DNA, 19 bp, Artificial Sequence
  Synthetic forward primer
ggcatcccagctccaactc

SEQ ID NO: 34 - DNA, 24 bp, Artificial Sequence
  Synthetic reverse primer
gggctctctccagtttacagatga

SEQ ID NO: 35 - DNA, 21 bp, Artificial Sequence
  Synthetic forward primer
caagtacggcctgttccaaca

SEQ ID NO: 36 - DNA, 18 bp, Artificial Sequence
  Synthetic reverse primer
cgcacaaagctgccatca

SEQ ID NO: 37 - DNA, 19 bp, Artificial Sequence
  Synthetic forward primer
cccaacacctggcatcatc

SEQ ID NO: 38 - DNA, 20 bp, Artificial Sequence
  Synthetic reverse primer
accaccccaaccgagaagag

SEQ ID NO: 39 - DNA, 19 bp, Artificial Sequence
  Synthetic forward primer
gcagcgaagaggctcaatg

SEQ ID NO: 40 - DNA, 21 bp, Artificial Sequence
  Synthetic reverse primer
gcagcgtcagcaaatgctact

SEQ ID NO: 41 - DNA, 24 bp, Artificial Sequence
  Synthetic forward primer
acccactgttcatcatgttctcat

SEQ ID NO: 42 - DNA, 19 bp, Artificial Sequence
  Synthetic reverse primer
cggaatgcaccatgcactt

SEQ ID NO: 43 - DNA, 18 bp, Artificial Sequence
  Synthetic forward primer
cgctcggcatggctatct

SEQ ID NO: 44 - DNA, 20 bp, Artificial Sequence
  Synthetic reverse primer
ctcgttgaagaacgcatcca

SEQ ID NO: 45 - DNA, 21 bp, Artificial Sequence
  Synthetic forward primer
aagccattcacttccccaatc

SEQ ID NO: 46 - DNA, 20 bp, Artificial Sequence
  Synthetic reverse primer
gcctcaccgtgcatgtttta

SEQ ID NO: 47 - DNA, 21 bp, Artificial Sequence
  Synthetic forward primer
ccagtcaactcctcgcacttt

SEQ ID NO: 48 - DNA, 20 bp, Artificial Sequence
  Synthetic reverse primer
tgaccgtgtccggtatttcc

SEQ ID NO: 49 - DNA, 15 bp, Artificial Sequence
  Synthetic forward primer
cgggcggcaggtttc

SEQ ID NO: 50 - DNA, 22 bp, Artificial Sequence
  Synthetic reverse primer
ctgggacaaggtgatgaacatg

SEQ ID NO: 51 - DNA, 18 bp, Artificial Sequence
  Synthetic forward primer
gaggaagcggcacatgga

SEQ ID NO: 52 - DNA, 24 bp, Artificial Sequence
  Synthetic reverse primer
tggtatggacacaaggtagaaagg

SEQ ID NO: 53 - DNA, 21 bp, Artificial Sequence
  Synthetic forward primer
ttttcaaggtcgggagtgatg

SEQ ID NO: 54 - DNA, 24 bp, Artificial Sequence
  Synthetic reverse primer
actttttcatatgccacctccttt

SEQ ID NO: 55 - DNA, 20 bp, Artificial Sequence
  Synthetic forward primer
tgggactcgaacggctattg

SEQ ID NO: 56 - DNA, 18 bp, Artificial Sequence
  Synthetic reverse primer
gaacaggcaccgcaccat

SEQ ID NO: 57 - DNA, 23 bp, Artificial Sequence
  Synthetic forward primer
gcagagtcgtaggaagcatttgt

SEQ ID NO: 58 - DNA, 18 bp, Artificial Sequence
  Synthetic reverse primer
tgggacgccgaaatcatg

SEQ ID NO: 59 - DNA, 23 bp, Artificial Sequence
  Synthetic forward primer
gatgaaggtggacgattgaattc

SEQ ID NO: 60 - DNA, 19 bp, Artificial Sequence
  Synthetic reverse primer
ccgttgccctgtgattacg

SEQ ID NO: 61 - DNA, 22 bp, Artificial Sequence
  Synthetic forward primer
ttggtctttcccctaacccttt

SEQ ID NO: 62 - DNA, 24 bp, Artificial Sequence
  Synthetic reverse primer
aactgccactctagcaagaattca

SEQ ID NO: 63 - DNA, 22 bp, Artificial Sequence
  Synthetic forward primer
gagggaaaatcctagcgagtca

SEQ ID NO: 64 - DNA, 22 bp, Artificial Sequence
  Synthetic reverse primer
agtccggcttctaccaatcaaa

SEQ ID NO: 65 - DNA, 21 bp, Artificial Sequence
  Synthetic forward primer
cactcccagggacttgtttcc

SEQ ID NO: 66 - DNA, 17 bp, Artificial Sequence
  Synthetic reverse primer
gccgacacgggttttcc

SEQ ID NO: 67 - DNA, 21 bp, Artificial Sequence
  Synthetic forward primer
cagctgcccaggaagataatg

SEQ ID NO: 68 - DNA, 16 bp, Artificial Sequence
  Synthetic reverse primer
ccccgctgtggctttg

SEQ ID NO: 69 - DNA, 27 bp, Artificial Sequence
  Synthetic forward primer
ctccaatgagcttctagagtgttatca

SEQ ID NO: 70 - DNA, 17 bp, Artificial Sequence
  Synthetic reverse primer
ggaagaccccggccaat

SEQ ID NO: 71 - DNA, 20 bp, Artificial Sequence
  Synthetic forward primer
gcggaatgaatggaaacgtt

SEQ ID NO: 72 - DNA, 21 bp, Artificial Sequence
  Synthetic reverse primer
ttgaggagaagcctggagtga

SEQ ID NO: 73 - DNA, 16 bp, Artificial Sequence
  Synthetic forward primer
gcacccgggcacacaa

SEQ ID NO: 74 - DNA, 20 bp, Artificial Sequence
  Synthetic reverse primer
aggcgatcaatccctgagaa

SEQ ID NO: 75 - DNA, 20 bp, Artificial Sequence
  Synthetic forward primer
tctgtctcggcagctgacat

SEQ ID NO: 76 - DNA, 24 bp, Artificial Sequence
  Synthetic reverse primer
accacaaaagatcaaggtgagtga

SEQ ID NO: 77 - DNA, 36 bp, Artificial Sequence
  Synthetic forward primer
acggaggttgtgaggcactgcccccaccatgagcgc

SEQ ID NO: 78 - DNA, 36 bp, Artificial Sequence
  Synthetic reverse primer
gcgctcatggtgggggcagtgcctcacaacctccgt

SEQ ID NO: 79 - DNA, 36 bp, Artificial Sequence
  Synthetic forward primer
aacagctttgaggtgcatgtttgtgcctgtcctggg

SEQ ID NO: 80 - DNA, 36 bp, Artificial Sequence
  Synthetic reverse primer
cccaggacaggcacaaacatgcacctcaaagctgtt

SEQ ID NO: 81 - DNA, 33 bp, Artificial Sequence
  Synthetic forward primer
atgggcggcatgaactggaggcccatcctcacc

SEQ ID NO: 82 - DNA, 33 bp, Artificial Sequence
  Synthetic reverse primer
ggtgaggatgggcctccagttcatgccgcccat

SEQ ID NO: 83 - DNA, 33 bp, Artificial Sequence
  Synthetic forward primer
atgggcggcatgaaccagaggcccatcctcacc

SEQ ID NO: 84 - DNA, 33 bp, Artificial Sequence
  Synthetic reverse primer
ggtgaggatgggcctctggttcatgccgcccat

SEQ ID NO: 85 - DNA, 33 bp, Artificial Sequence
  Synthetic forward primer
agttcctgcatgggctccatgaaccggaggccc

SEQ ID NO: 86 - DNA, 33 bp, Artificial Sequence
  Synthetic reverse primer
gggcctccggttcatggagcccatgcaggaact

SEQ ID NO: 87 - DNA, 45 bp, Artificial Sequence
  Synthetic forward primer
ctctgagtcaggaaacattttcagaccaatcgaaactacttcctg

SEQ ID NO: 88 - DNA, 45 bp, Artificial Sequence
  Synthetic reverse primer
caggaagtagtttcgattggtctgaaaatgtttcctgactcagag

SEQ ID NO: 89 - DNA, 43 bp, Artificial Sequence
  Synthetic forward primer
ccccggacgatattgaacaacagtccactgaagacccaggtcc

SEQ ID NO: 90 - DNA, 43 bp, Artificial Sequence
  Synthetic reverse primer
ggacctgggtcttcagtggactgttgttcaatatcgtccgggg

(19) United States
(12) Patent Application Publication
(10) Pub. No.: US 2013/0281493 A1
(43) Pub. Date: Oct. 24, 2013

(54) Method for Treating Cancer Harboring a p53 Mutation

(71) Applicants: William Allen Freed-Pastor, Canfield, OH (US); Carol Prives, Greenlawn, NY (US)

(72) Inventors: William Allen Freed-Pastor, Canfield, OH (US); Carol Prives, Greenlawn, NY (US)

(73) Assignee: The Trustees of The University of Columbia in the City of New York, New York, NY (US)

(21) Appl. No.: 13/878,300

(22) PCT Filed: Oct. 7, 2011
(86) PCT No.: PCT/US11/55488

§ 371 Date: Jul. 8, 2013

Related U.S. Application Data
(60) Provisional application No. 61/391,068, filed on Oct. 7, 2010.

(51) Int. Cl. A61K 31/4439; A61K 31/366

(52) U.S. Cl. CPC A61K 31/4439; A61K 31/366
USPC 514/342; 435/7.1; 514/460; 549/292; 506/9; 435/6.12; 546/270.4

(57) ABSTRACT

A method for determining if a subject with cancer or precancerous lesions or a benign tumor, will respond to treatment with an inhibitor selected from the group comprising an inhibitor of one or more enzymes in the mevalonate pathway, an inhibitor of geranylgeranyl transferase, an inhibitor of farnesyl transferase or an inhibitor of squalene synthase, by (i) obtaining a sample of the cancer cells, precancerous cells or benign tumor cells from the subject, (ii) assaying the cells in the sample for the presence of a mutated p53 gene or a mutant form of p53 protein or a biologically active fragment thereof, and (iii) if the cells have the mutated p53 gene or mutant form of the p53 protein, then determining that the subject will respond to treatment with the inhibitor or combinations thereof. Some embodiments are directed to treatment with the inhibitors.

### CROSS-REFERENCE TO RELATED APPLICATIONS

This application claims benefit of Provisional Application No. 61/391,068, filed Oct. 7, 2010, and is a 371 application of PCT/US11/55488, filed Oct. 7, 2011, the entire contents of which are hereby incorporated by reference as if fully set forth herein, under 35 U.S.C. §119(e).

### STATEMENT OF GOVERNMENTAL INTEREST

This invention was made with Government support under Contract No. NCI CA87497 awarded by NIH HHS/United States. The Government has certain rights in the invention.

### BACKGROUND

The TP53 gene, which encodes the p53 protein, is the most frequent target for mutation in tumors, with over half of all human cancers exhibiting mutation at this locus (Vogelstein et al., 2000). Wild-type p53 functions primarily as a transcription factor and possesses an N-terminal transactivation domain, a centrally located sequence specific DNA binding domain, followed by a tetramerization domain and a C-terminal regulatory domain (Laptenko and Prives, 2006). In response to a number of stressors, including DNA damage, hypoxia and oncogenic activation, p53 becomes activated to promote cell cycle arrest, apoptosis or senescence thereby suppressing tumor growth. It also plays many additional roles including regulating cellular metabolism (Muller et al., 2009).

Unlike most tumor suppressor genes, which are predominantly inactivated as a result of deletion or truncation, the majority of mutations in TP53 are missense mutations, a few of which cluster at “hotspot” residues in the DNA binding core domain (Petitjean et al., 2007), while the N- and C-terminal domains of this protein are relatively spared from mutation (Hussain and Harris, 1998; Soussi and Lozano, 2005; Unger et al., 1993). In contrast to wild-type p53, which under unstressed conditions is a very short-lived protein, these missense mutations lead to the production of full-length p53 protein with a prolonged half-life (Davidoff et al., 1991; Rotter, 1983). While many tumor-derived mutant forms of p53 can exert a dominant-negative effect on the remaining wild-type allele, serving to abrogate the ability of wild-type p53 to inhibit cellular transformation, the end result in many forms of human cancer is frequently loss of heterozygosity (LOH), where the wild-type version of p53 is lost and the mutant form is retained, suggesting that there is a selective advantage conferred by losing the remaining wild-type p53, even after one allele has been mutated (Brosh and Rotter, 2009).

There is substantial evidence that certain mutants of p53 can exert oncogenic, or gain-of-function, activity independent of their effects on wild-type p53. In vivo models, in which mice harboring two tumor-derived mutants of p53 (equivalent to R175H and R273H in humans) that were substituted for the endogenous wild-type p53 locus within the mouse genome, display an altered tumor spectrum as well as more metastatic tumors (Lang et al., 2004; Olive et al., 2004). The mutational status of p53 has been shown to predict poor outcomes in multiple types of human tumors, including breast cancer, and certain mutants of p53 associate with an even worse prognosis (Olivier et al., 2006; Petitjean et al., 2007). Mutant p53 has also been demonstrated to lead to increased survival, invasion, migration and metastasis in preclinical breast cancer models (Adorno et al., 2009; Muller et al., 2009; Stambolsky et al., 2010). Despite these findings, mutant p53-induced phenotypic alterations in mammary tissue architecture have not been fully explored.

The association between mutated p53 protein and TP53 and cancer has been widely studied for most tumor sites in most human ethnic groups (Varley, Hum Mutat 2003; 21:313-20; Royds et al. Cell Death Differ 2006; 13:1017-26, Savage et al. Pediatr Blood Cancer 2007; 49:28-33, Ueda et al. Gynecol Oncol 2006; 100:173-8; Ignaszak-Szczepaniak et al. Oncol Rep 2006; 16:65-7; Wang-Gohrke et al. Br J Cancer 1999; 81:179-83; Wu et al. Cancer Res 2006; 66:8287-92). Different single polymorphisms and haplotypes are associated with different risk increments. The risk for Li-Fraumeni syndrome (multisite cancer syndrome) that involves a germline mutation in p53 increases risk of cancer 100-fold for men and 1000-fold for women. Thus, there is a great need for methods of treating cancer having mutated p53 protein and TP53.

### BRIEF DESCRIPTION OF THE DRAWINGS

FIG. 1. Depletion of mutant p53 from breast cancer cells induces a phenotypic reversion in 3D culture: (A) Depletion of mutant p53 dramatically affects 3D morphology of MDA-231 cells. MDA-231.shp53 cells were grown under 3D conditions for 8 days in the absence of DOX, thus retaining full levels of mutant p53, or grown in the presence of DOX to knockdown endogenous mutant p53. Representative differential interference contrast (DIC) images are shown. Scale Bar, 200 μm. (B) shRNA mediated reduction of mutant p53 in MDA-231 cells. MDA-231.shp53 cells were grown in 3D culture for 8 days in the presence or absence of DOX as indicated prior to lysis and immunoblotting analysis as in Methods. p53 was detected using anti-p53 antibody (PAb1801). Actin serves as a loading control. (C) Morphologic categories in MDA-468 cells. MDA-468.shp53 cells were grown in 3D cultures for 8 days and structures were grouped into three morphological categories: Malignant, Intermediate and Hollow Lumen. Actin cytoskeleton was stained with Phalloidin (Green) and nuclei were stained with DRAQ5 (Red). Structures were analyzed by confocal microscopy. Scale bar, 50 μm. (D) Depletion of mutant p53 induces a phenotypic reversion in MDA-468 cells. MDA-468.shp53 cells were grown in 3D cultures for 8 days in the presence of DOX, leading to induction of an shRNA targeting p53, and thus to depleted levels of mutant p53. Left panel: GFP (Green) serves as a marker for shRNA induction. Right panel: Nuclei were stained with DRAQ5 (Red) and analyzed by confocal microscopy. The larger structure is representative of intermediate colony morphologies, while the smaller structure is representative of acinus-like structures with hollow lumen morphology. White arrow indicates cell debris from apoptosis within the luminal space. Scale bar, 50 μm. (E) Knockdown of mutant p53 in MDA-468 cells. MDA-468.shp53 cells were grown in 3D culture for 8 days in the presence or absence of DOX as indicated and processed as in (B). p53 was detected using an anti-p53 antibody (PAb1801). Actin serves as a loading control. (F) Morphometry of MDA-468.shp53 pooled population. A stable pool of MDA-468.shp53 cells were grown in 3D cultures for 8 days in the presence or absence of DOX as indicated and structures were analyzed by confocal microscopy and categorized as in (C). Left panel illustrates population distribution. Right panel shows the percent of acinus-like structures with hollow lumens without (−) or with (+) mutant p53 depletion by treatment with DOX. Structures (50-100) were counted for each condition. *denotes p<0.01. (G) Morphometry of MDA-468.shp53 clonal population. A stable clone of MDA-468.shp53 cells were grown in 3D cultures for 8 days in the presence or absence of DOX as indicated and structures were analyzed by confocal microscopy as in (F). Structures (50-100) were counted for each condition and plotted as a percentage of the population. An average of two experiments is shown.

FIG. 2. Mutant p53 requires functional transactivation sub-domains to disrupt morphology of mammary cells in 3D culture: (A) MDA-468.shp53 cells expressing a control vector (pLNCX) were grown in 3D cultures for 5 days in the absence of DOX thus retaining full levels of mutant p53 (left panel), or grown in the presence of DOX inducing an shRNA that targets p53 (right panel), leading to depleted levels of mutant p53 as in FIG. 1 or in (D) below. Representative DIC images are shown. Scale bar, 200 μm. (B) MDA-468.shp53 cells expressing an shRNA-resistant Flag-tagged p53-R273H were grown in 3D cultures for 5 days in the absence or presence of DOX as in (A). Representative DIC images are shown. Scale bar, 200 μm. (C) MDA-468.shp53 cells expressing an shRNA-resistant Flag-tagged p53-R273H-mTD (mutant p53 with non-functional transactivation region) were grown in 3D cultures for 5 days in the absence or presence of DOX as in (A). Representative DIC images are shown. Scale bar, 200 μm. (D) Immunoblot of mutant p53 in MDA-468 cells. Cells either with control vector or expressing shRNA resistant versions of p53-R273H mutant p53 or transactivation defective p53-R273-mTD were grown in 3D culture for 5 days in the absence or presence of DOX as indicated followed by lysis and processing for immunoblotting as in FIG. 1B. p53 was detected using an anti-p53 antibody (PAb240). Note that exogenously expressed tagged mutant p53 variants migrate more slowly than endogenously expressed mutant p53. Actin serves as a loading control.

FIG. 3. Knockdown of mutant p53 from breast cancer cells in 3D culture significantly downregulates the mevalonate pathway: (A) Pathway analysis of breast cancer cells following mutant p53 depletion. Data were analyzed through the use of Ingenuity Pathways Analysis (Ingenuity® Systems, www.ingenuity.com). Significant (p<0.01) expression changes from genome-wide expression analysis were queried. Blue bars that cross the threshold line (p<0.05) represent pathways that are significantly changed following mutant p53 depletion from MDA-468 cells. (B) Biological processes significantly altered by mutant p53 in 3D culture. Significant expression changes from genome-wide expression analysis were analyzed by Gene Ontology (GO) analysis. 1, 2, 3 represent three independent experiments. GO terms were sorted based their significance and redundant terms were discarded. (C) Validation of sterol biosynthesis genes. MDA-468.shp53 cells were grown in 3D cultures for 8 days in the presence or absence of DOX as indicated to deplete cells of mutant p53. Isolated RNA was reverse transcribed and qRT-PCR was performed for the seven sterol biosynthesis genes identified by Ingenuity Pathway Analysis: HMGCR, HMG-CoA reductase; MVK, Mevalonate Kinase; MVD, Mevalonate Decarboxylase; FDPS, Farnesyl Diphosphate Synthase; SQLE, Squalene Epoxidase; LSS, Lanosterol Synthase; DHCR7, 7-Dehydrocholesterol reductase. Data is presented as mean±st dev of three independent experiments. **indicates p<0.005 by two-sided t-test.

FIG. 4. Downregulation of the mevalonate pathway is both necessary and sufficient to induce phenotypic reversion of breast cancer cells in 3D culture to a normal phenotype: (A) Intermediate metabolites rescue the phenotypic effects of depleting breast cancer cells of mutant p53. MDA-468.shp53 cells were grown in 3D cultures for 8 days in the presence or absence of DOX to deplete mutant p53. Parallel wells of cells which were grown in the presence of DOX were supplemented with metabolites produced within the mevalonate pathway: mevalonic acid/mevalonic acid-phosphate (MVA/MVAP) beginning on Day 1. Morphological categories as indicated were determined for 50-100 structures using confocal microscopy which were then plotted as a percentage of the population. A representative experiment is shown here and a second representative experiment is shown in FIG. 10.

(B) Inhibition of the mevalonate pathway affects MDA-468 cell morphology in 3D cultures. MDA-468 cells were grown in 3D culture conditions for 13 days untreated or treated with vehicle (DMSO), Simvastatin (100 nM) or Simvastatin (1 μM) as indicated. Drugs were added on Day 4. Scale Bar, 200 μm. (C) Inhibition of the mevalonate pathway affects MDA-231 cell morphology in 3D cultures. MDA-231 cells were grown in 3D culture conditions for 13 days untreated or treated with vehicle (DMSO), Simvastatin (100 nM) or Simvastatin (1 μM) as indicated. Drugs were added on Day 4. Scale Bar, 200 μm. (D) The effects of Simvastatin are due to inhibition of HMG-CoA reductase. MDA-468 cells (top panel) or MDA-231 cells (bottom panel) were grown in 3D cultures for 13 days with Simvastatin (1 μM) as in (B) and (C), respectively, but were supplemented with mevalonic acid/mevalonic acid-phosphate, the early enzymatic products after HMG-CoA reductase. Scale Bar, 200 μm, (E) Supplementation with mevalonic acid is sufficient to block luminal clearance in MCF10A cells. MCF10A cells were grown in 3D culture for 8 days in the absence (Control) or presence (MVA) of 1 mM mevalonic acid. Nuclei were stained with DRAQ5 (Blue) and structures were analyzed by confocal microscopy for the presence of a hollow or filled lumen (right panel). Structures (50-100) were counted for each condition. An average of two experiments is presented. Scale Bar, 50 μm.

FIG. 5. Geranylgeranylation mediates many of the phenotypic effects of mutant p53 depletion and HMG-CoA reductase inhibition: (A) Inhibition of downstream enzymes in the mevalonate pathway affects MDA-231 cell morphology in 3D cultures. MDA-231 cells were grown in 3D culture conditions for 8 days untreated or treated with vehicle (DMSO), YM-53601 (1 μM), FTI-277 (1 μM) or GGTI-2133 (1 μM) as indicated. Drugs were added on Day 1. Scale Bar, 200 μm. (B) Geranylgeranyl pyrophosphate can partially rescue the morphological effects of mutant p53 depletion. MDA-231.shp53 cells were grown in 3D culture conditions for 8 days in the absence (−DOX) or presence (+DOX) of doxycycline as indicated. Parallel wells of cells which were grown in the presence of DOX were supplemented with geranylgeranyl pyrophosphate (GGPP) beginning on Day 1. Scale Bar, 200 μm. (C) Geranylgeranyl pyrophosphate can partially rescue the morphological effects of HMG-CoA reductase inhibition. MDA-231 cells were grown in 3D culture conditions for 8 days either treated with vehicle (DMSO) or Simvastatin (1 μM) as indicated. Parallel wells of cells which were grown in the presence of Simvastatin (1 μM) were supplemented with geranylgeranyl pyrophosphate (GGPP) beginning on Day 1. Scale Bar, 200 μm.

FIG. 6. Mutant p53 is correlated with higher expression of a subset of sterol biosynthesis genes in human breast cancer patient datasets: (A) Five human breast cancer patient datasets were analyzed to determine whether tumors bearing mutant p53 correlate with higher expression of sterol biosynthesis genes. Patients were stratified based on TP53 status (wild-type vs. mutant) and expression levels for sterol biosynthesis genes were analyzed. One of the significantly associated genes, Isopentenyl Pyrophosphate Isomerase (IDI1), exhibited higher expression levels in mutant p53 tumors compared to wild-type p53 tumors (p<0.05) across all five datasets. p-value represents the result of a one-sided t-test. See Table 1 for all genes. (B) Unsupervised hierarchical clustering with Euclidean distance and ward linkage of expression matrix from the 17 sterol biosynthesis genes on 812 samples. MVD was not present in the DBCG dataset and its missing expression values were grayed out on the heatmap. (C) The Kaplan-Meier curves for the resulting clusters from the unsupervised hierarchical clustering in (B). (D) Estimated hazard ratios (HRs; the relative risk for 1 unit increasing in the gene expression) with 95% confidence interval for risk of breast cancer specific death. Expression levels of following genes were positively associated with the risk of breast cancer specific death at FDR 5%: ACAT2 (HR=1.23, q=0.0069), HMGCS1 (HR=1.21, q=0.007), HMGCR(HR=1.17, q=0.032), IDI1 (HR=1.26, q<0.001), FDPS(HR=1.17, q=0.012), SQLE (HR=1.35, q<0.001), LSS (HR=1.16, q=0.032), NSDHL (HR=1.17, q=0.032), DHCR7 (HR=1.26, q<0.001).

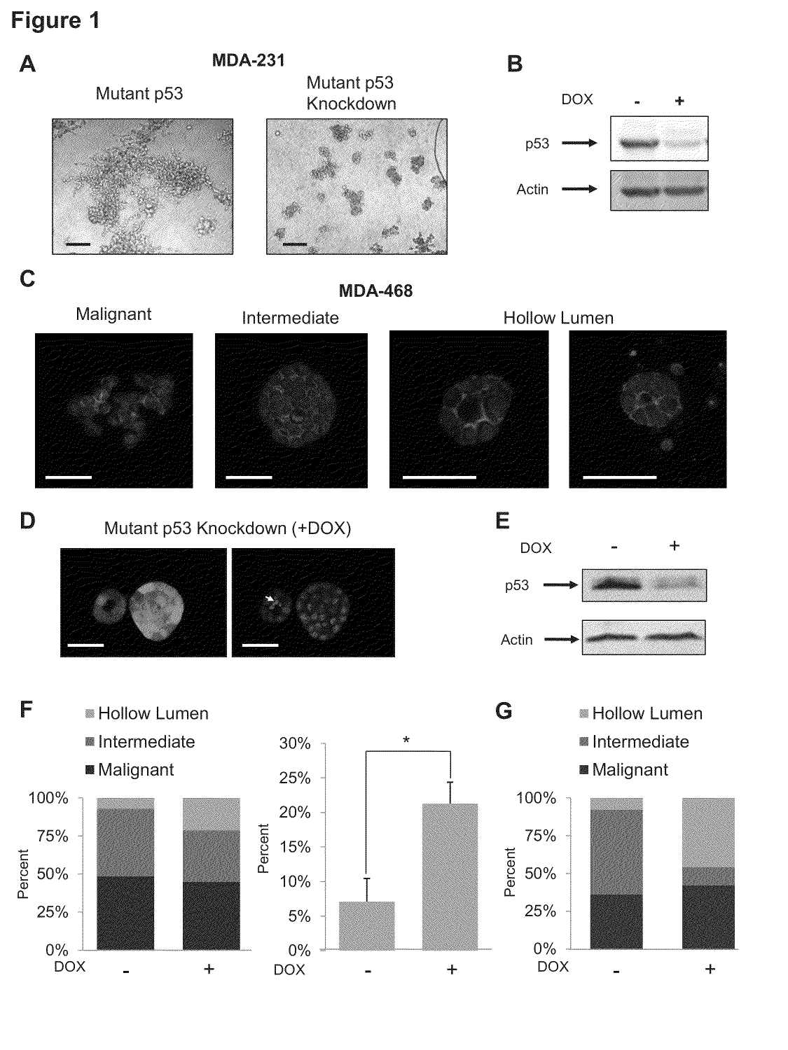

FIG. 7. Depletion of mutant p53 from breast cancer cells induces a phenotypic reversion in 3D culture (Related to FIG. 1): (A) Doxycycline curve in MDA-231.shp53 cells. MDA-231.shp53 cells were grown in 2D culture in the presence of the indicated concentrations of DOX for 8 days. p53 was detected using an anti-p53 antibody (PAb1801). Actin serves as a loading control. (B) Doxycycline curve in MDA-231.shp53 cells in 3D culture. MDA-231.shp53 cells were grown in 3D culture for 8 days in the presence of the indicated concentrations of DOX and imaged using differential interference microscopy. Scale Bar, 200 μm. (C) Doxycycline curve in MDA-468.shp53 cells. MDA-468.shp53 cells were grown in 2D culture in the presence of the indicated concentrations of DOX for 8 days. p53 was detected using an anti-p53 antibody (PAb1801). Actin serves as a loading control. (D) Doxycycline curve in MDA-468.shp53 cells in 3D culture. MDA-468.shp53 cells were grown in 3D culture for 8 days in the presence of the indicated concentrations of DOX and imaged using differential interference microscopy. Scale Bar, 200 μm. (E) Reverted MDA-468.shp53 cells regain proper localization of a6 integrin. MDA-468.shp53 cells well grown in 3D culture for 8 days in the presence (top panels) or absence (bottom panels) of DOX to deplete levels of endogenous mutant p53. Alpha 6 integrin (red) was immunostained using a monoclonal antibody directed against a6 integrin and nuclei were stained with DRAQ5 (Blue). Structures were analyzed by confocal microscopy. Scale bar, 50 μm. (F) Expression of shRNA-resistant p53-R273H can compensate for depletion of p53-R280K from MDA-231 cells. MDA-231.shp53 cells expressing a control vector were grown in 3D culture for 8 days in the absence of DOX (top left panel), thus retaining full levels of mutant p53, or grown in the presence of DOX (top right panel) to ablate endogenous mutant p53. MDA-231.shp53 cells expressing a shRNA-resistant Flag-tagged version of mutant p53 (p53-R273H) were grown in 3D culture for 8 days in the absence of DOX (bottom left panel), thus retaining full levels of both exogenous and endogenous mutant p53, or grown in the presence of DOX (bottom right panel) to ablate endogenous mutant p53, but retain exogenous p53-R273H. Scale Bar, 200 μm. (G) Levels of endogenous mutant p53 and retention of exogenous mutant p53. Cells were grown for 8 days in the presence or absence of DOX as indicated. p53 was detected using an anti-p53 antibody (PAb1801). Actin serves as a loading control.

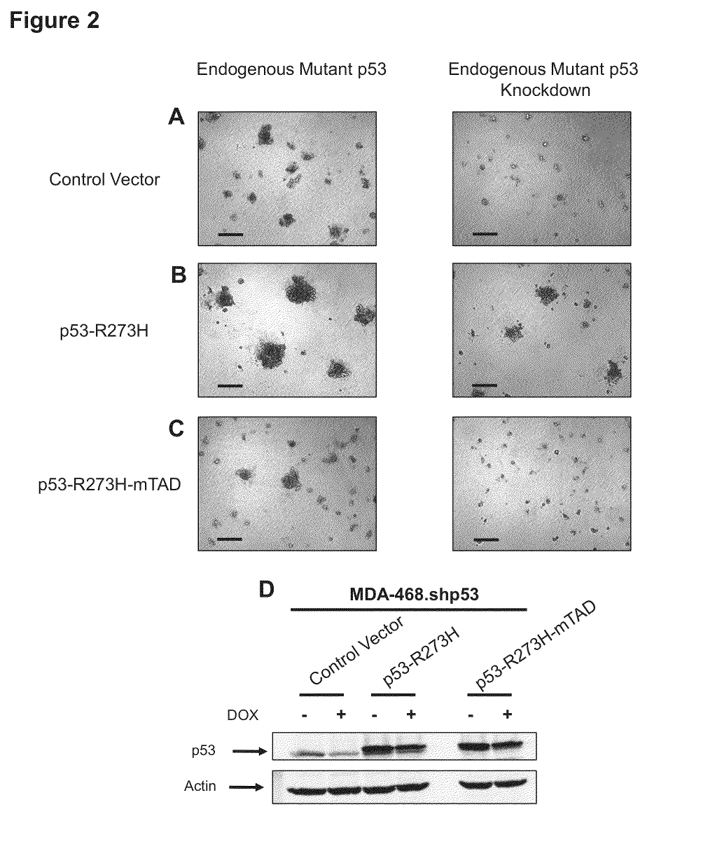

FIG. 8 Tumor-derived mutants of p53 disrupt acinar morphogenesis in non-malignant mammary epithelial cells. (Related to FIG. 2): (A) Schematic of normal mammary acinar development. (B-G) Mutant p53 disrupts normal mammary morphogenesis. MCF10A cells expressing an empty vector (B) or Flag-tagged versions of p53-R175H(C), p53-R273H (D), p53-R248W (E), p53-R248Q (F) or p53-G245S (G) were grown in 3D culture for 8 days. Structures were analyzed by confocal microscopy. Nuclei were stained with DRAQ5 (Red). Scale Bar, 50 μm. (H) Immunoblot for p53 expression demonstrating endogenous wild-type p53 and exogenous Flag-tagged mutants. MCF10A cells expressing Flag-tagged versions of mutant p53 were grown in 3D culture for 8 days. p53 was detected using an anti-p53 antibody (PAb1801). Actin serves as a loading control. (I) Morphometry of structures. MCF10A cells expressing tumor-derived mutants of p53, or their transactivation-deficient (mTAD) equivalents, were grown in 3D culture for 8 days and analyzed by confocal microscopy for the presence of a hollow or filled lumen. Structures (50-100) were counted for each condition. A representative experiment is shown. (J) Immunoblot for p53 expression demonstrating exogenous Flag-tagged mutants. MCF10A cells expressing Flag-tagged versions of mutant p53 were grown in 2D culture, lysed and whole cell extracts were subjected to SDS-PAGE and then immunoblotted. Exogenous p53 was detected using an anti-Flag antibody. Actin serves as a loading control.

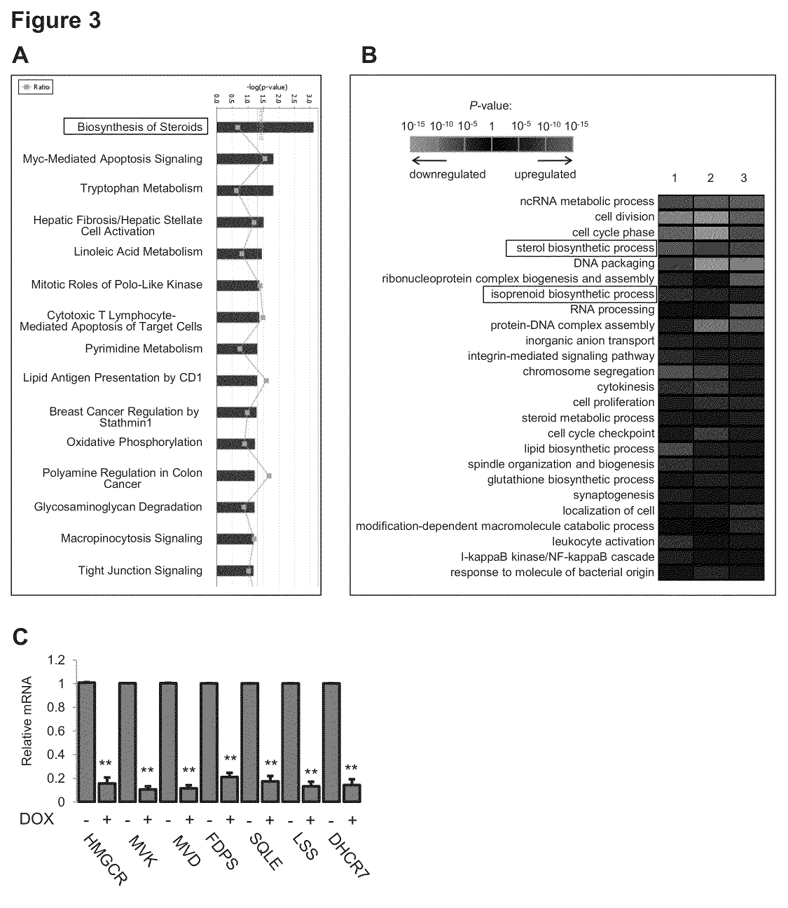

FIG. 9. Schematic of the mevalonate pathway, (Related to FIG. 3): (A) Schematic of the mevalonate pathway. Key intermediate metabolites are shown in bold. Gene names are shown in parentheses. Inhibitors are indicated using gray boxes.

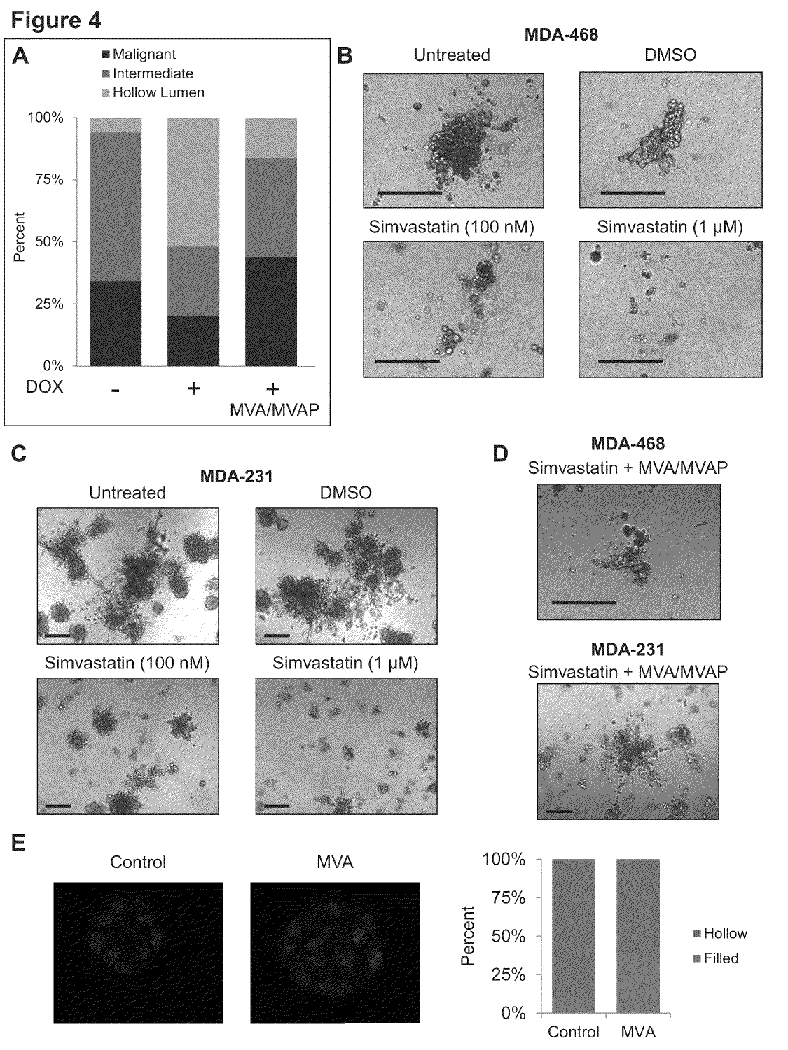

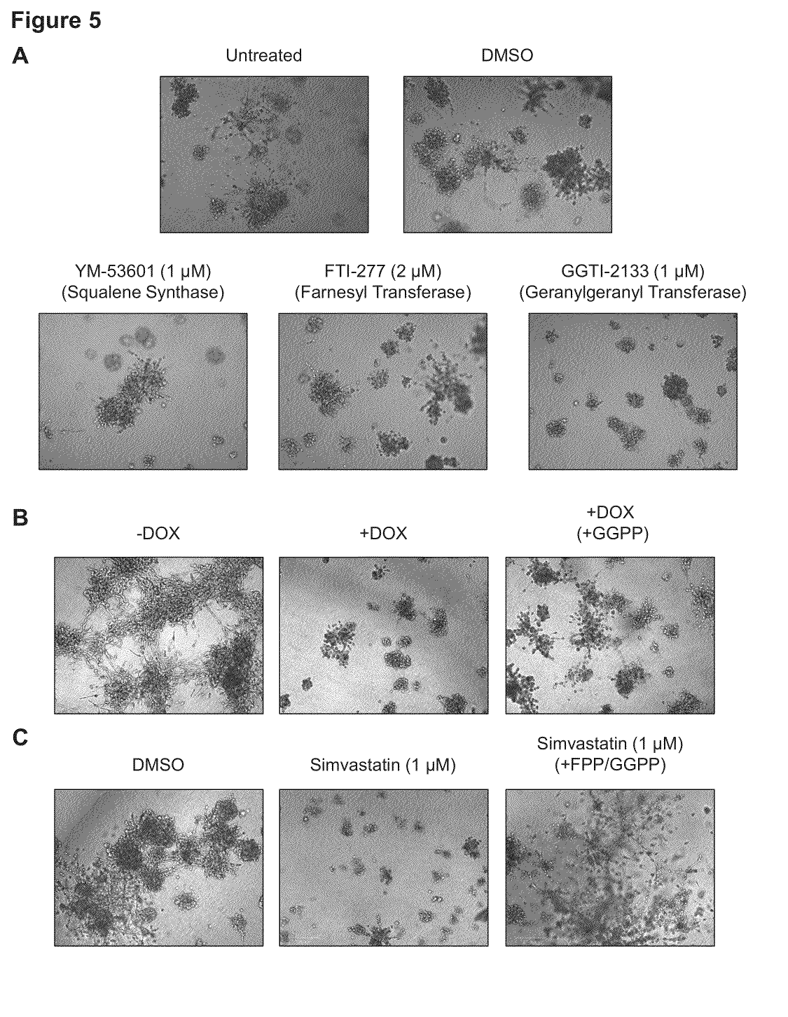

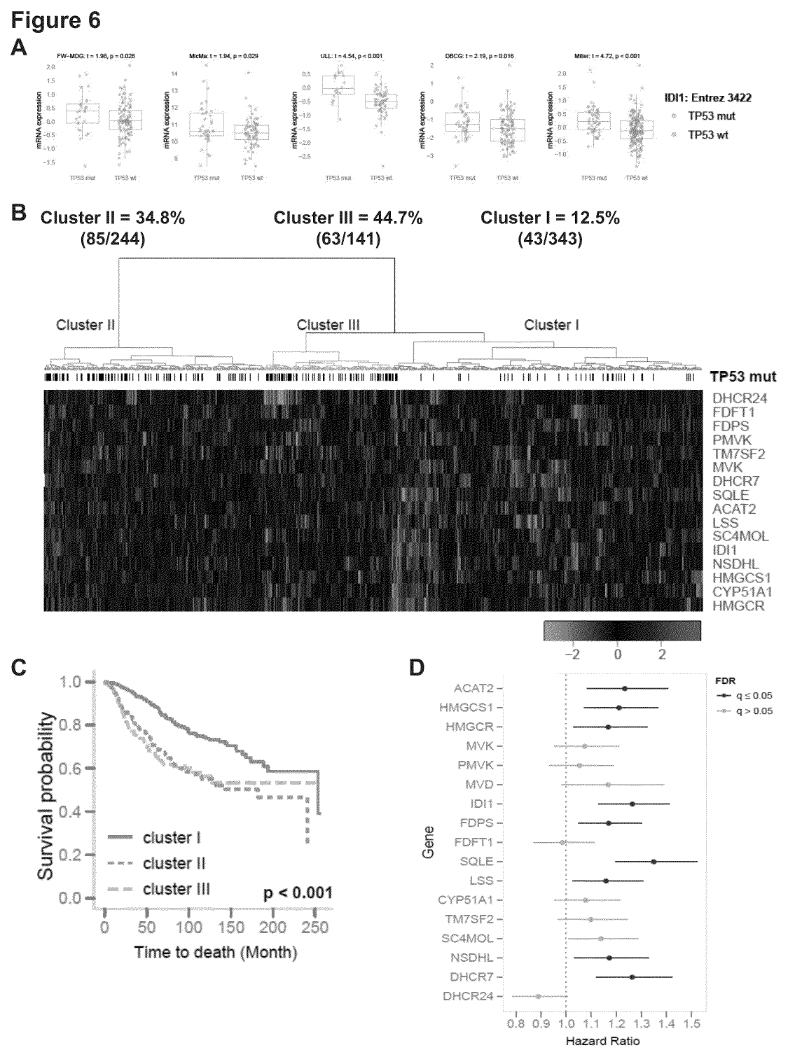

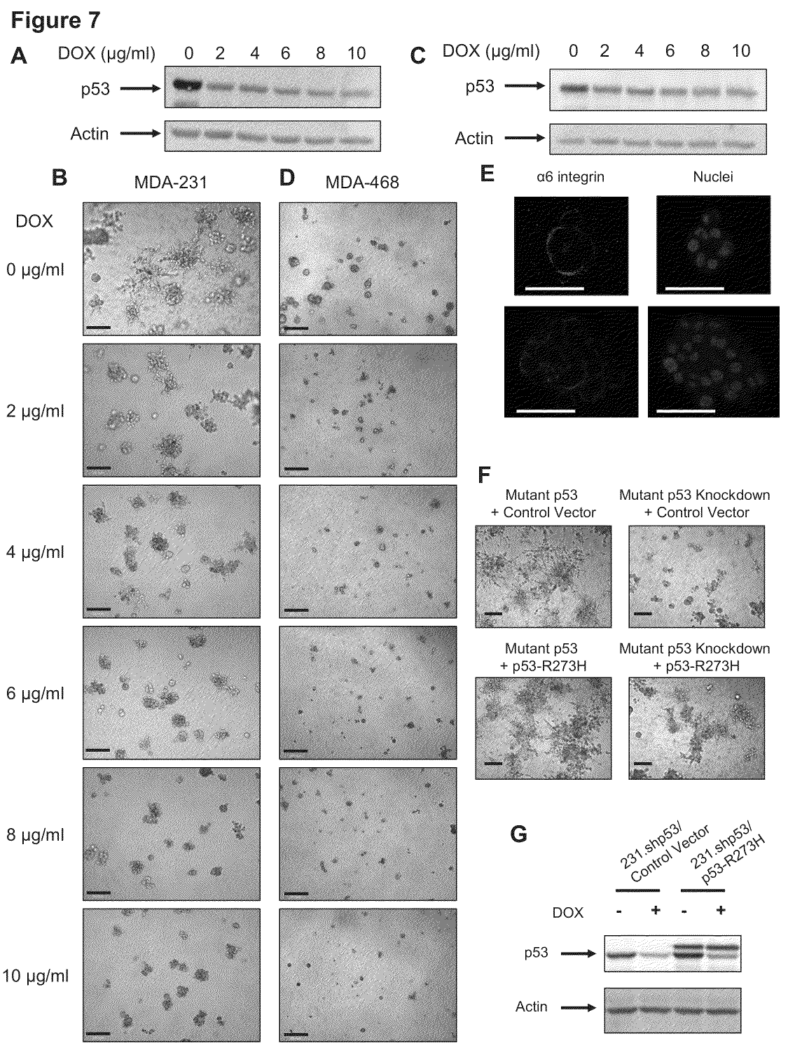

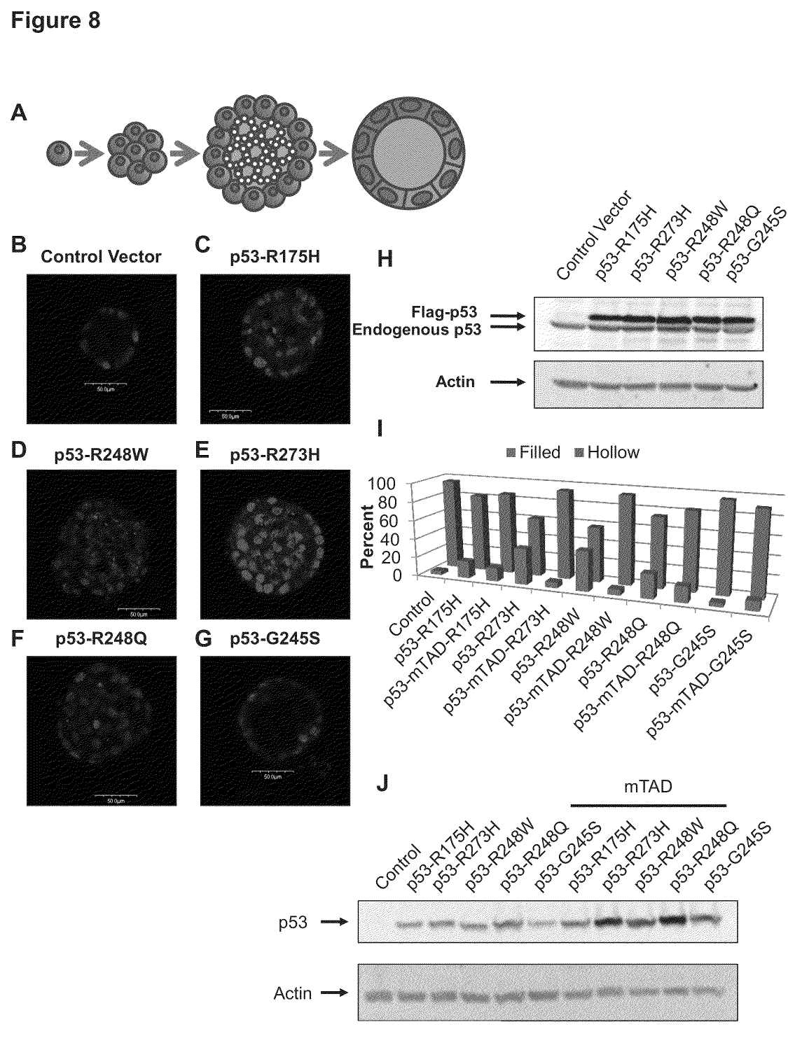

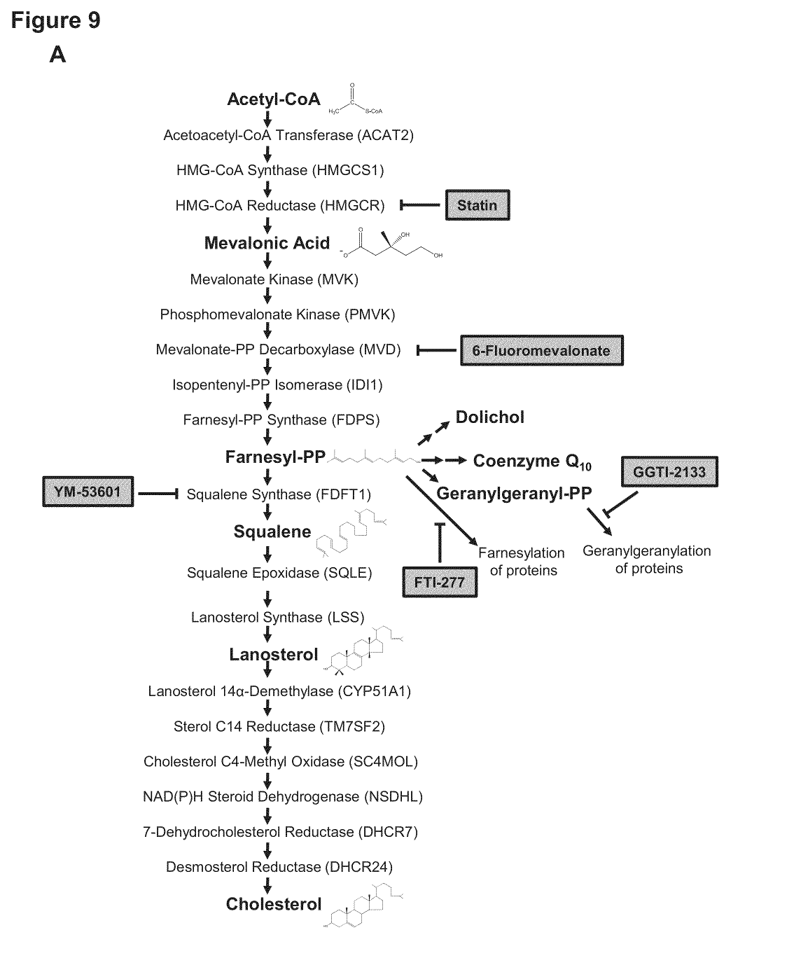

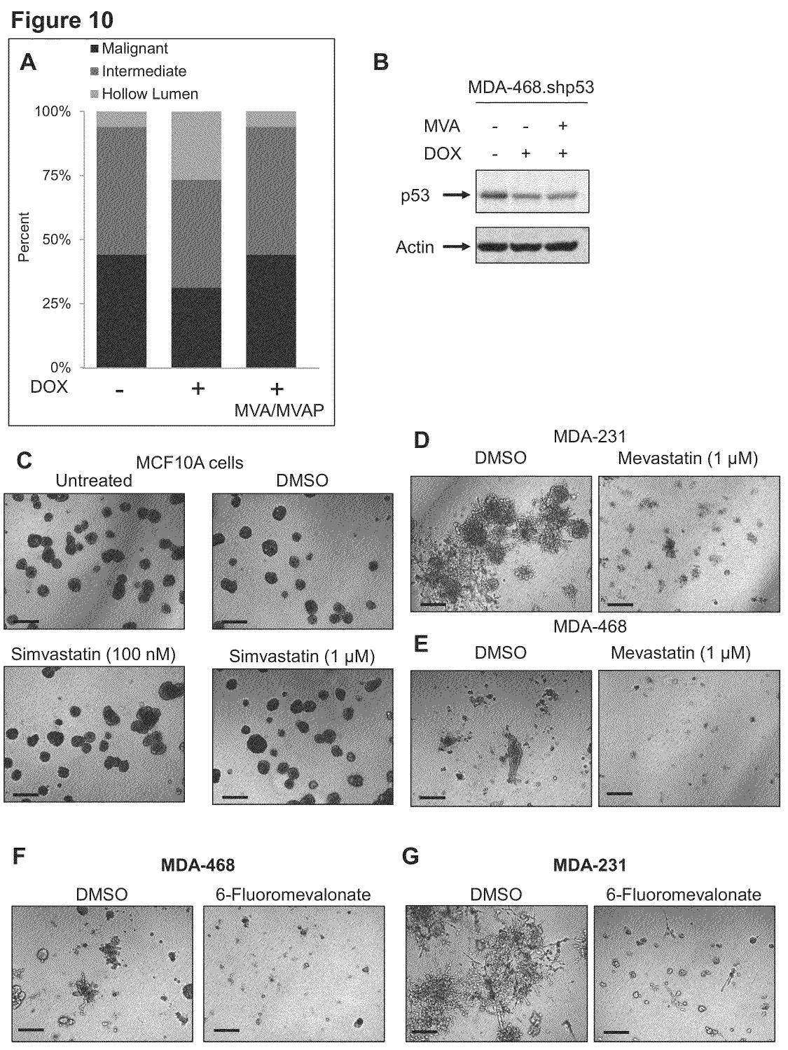

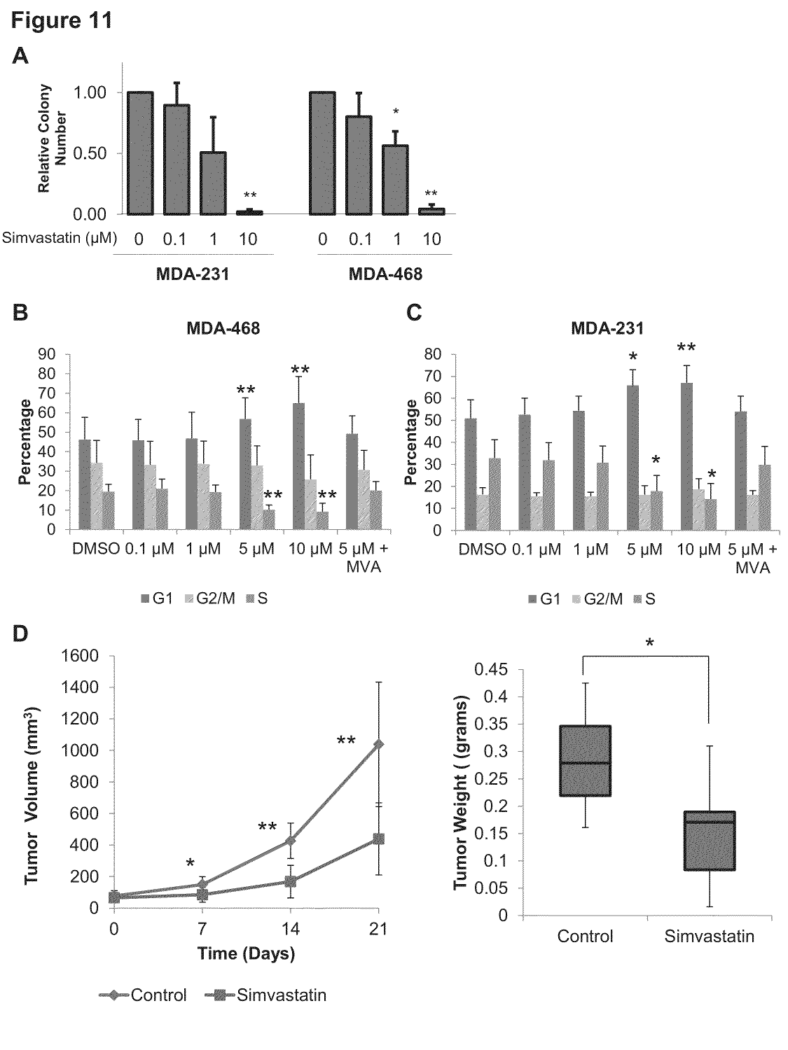

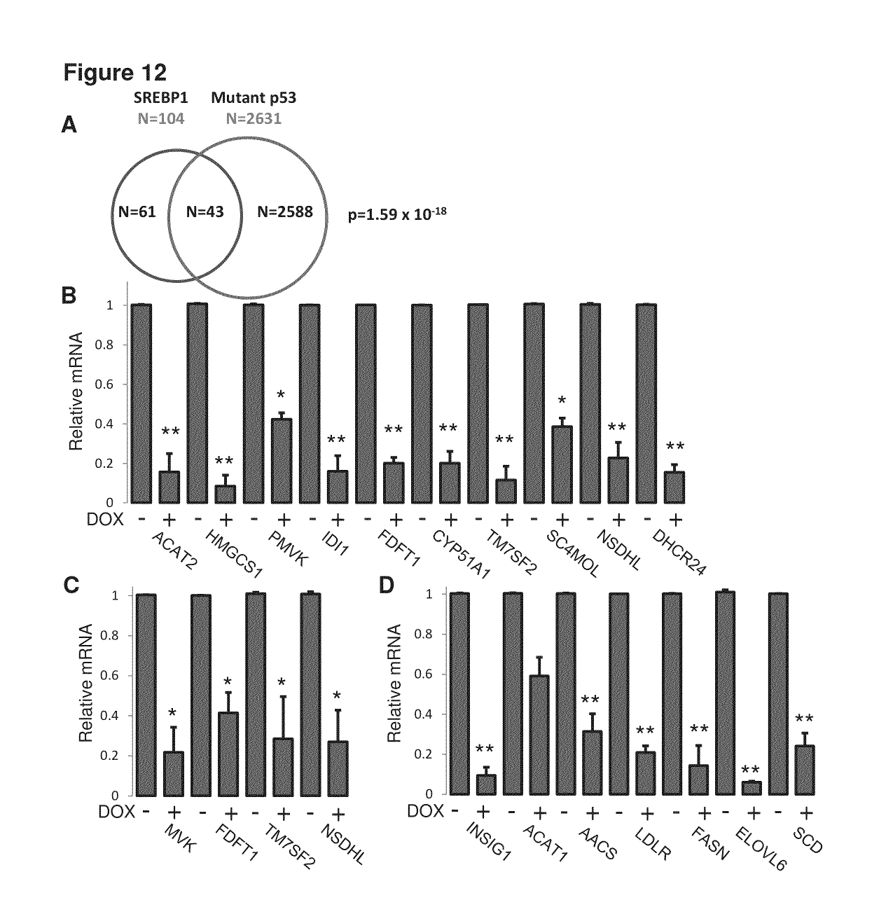

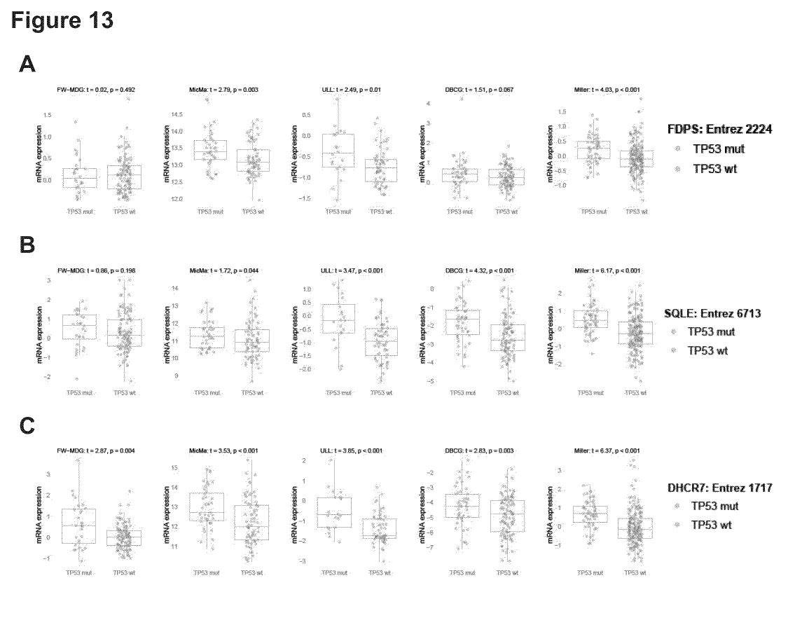

FIG. 10 Downregulation of the mevalonate pathway is both necessary and sufficient to phenotypically revert breast cancer cells in 3D culture, (Related to FIG. 4): (A) Intermediate metabolites rescue the phenotypic effects of depleting breast cancer cells of mutant p53. MDA-468.shp53 cells were grown in 3D culture for 8 days in the presence or absence of DOX to knockdown mutant p53. Cells which were grown in the presence of DOX were then supplemented with two metabolites produced within the mevalonate pathway: mevalonic acid/mevalonic acid-phosphate (MVA/MVAP). Morphological categories were determined for 50-100 structures using confocal microscopy which were then plotted as a percentage of the population. A representative experiment is shown. (B) Add-back of mevalonic acid/mevalonic acid-phosphate (MVA/MVAP) does not affect mutant p53 depletion by doxycycline. MDA-468.shp53 cells were cultured in the presence of doxycycline to knockdown mutant p53 with or without supplementation of 1 mM MVA/MVAP. Whole cell extracts were then subjected to SDS-PAGE and then immunoblotted. p53 was detected using an anti-p53 antibody (PAb1801). Actin serves as a loading control. (C) Simvastatin treatment does not affect the morphology of MCF10A cells. MCF10A cells were grown in 3D culture for 13 days untreated or treated with vehicle (DMSO), Simvastatin (100 nM) or Simvastatin (1 μM) as indicated. Drugs were added on Day 4. Scale Bar, 200 μm. (D) Mevastatin profoundly affects the 3D morphology of MDA-231 cells. MDA-231 cells were grown in 3D culture for 13 days. On Day 4, vehicle (DMSO) or Mevastatin (1 μM) were added as indicated for the remainder of the experiment. Scale Bar, 200 μm. (E) Mevastatin profoundly affects the 3D morphology of MDA-468 cells. MDA-468 cells were grown in 3D culture for 13 days. On Day 4, vehicle (DMSO) or Mevastatin (1 μM) were added as indicated for the remainder of the experiment. Scale Bar, 200 μm. (F) Inhibition of Mevalonate Decarboxylase affects the 3D morphology of MDA-468 cells. MDA-468 cells were grown in 3D cultures for 8 days. On Day 1, vehicle (DMSO) or 6-Fluoromevalonate (200 μM) was added for the remainder of the experiment. Scale Bar, 200 μm. (G) Inhibition of Mevalonate Decarboxylase affects the 3D morphology of MDA-231 cells. MDA-231 cells were grown in 3D cultures for 8 days. On Day 1, vehicle (DMSO) or 6-Fluoromevalonate (200 μM) was added for the remainder of the experiment. Scale Bar, 200 μm.

FIG. 11. Simvastatin prevents growth in breast cancer cells in vivo and induces a cell cycle arrest in cells grown in 2D culture, (Related to FIG. 5):

(A) Simvastatin prevents anchorage-independent growth in breast cancer cells. MDA-231 or MDA-468 cells were grown in soft-agar for 21 days in the presence of DMSO vehicle control (0 μM) or presence of Simvastatin (0.1, 1, 10 μM). Plates were subsequently stained with crystal violet and colonies were counted for each condition. Quantitation of three independent experiments illustrating relative colony number for MDA-468 (left) and MDA-231 (right) cells. Data presented as mean±st dev. *denotes p<0.05, **denotes p<0.01 using a two-tailed students t-test. (B-C) Simvastatin induces a G1 arrest in breast cancer cells grown in 2D culture. Flow cytometric analysis of cell cycle distribution for three independent experiments in MDA-468 cells (B) or MDA-231 cells (C). Data presented as mean±st dev. *denotes p<0.05, **denotes p<0.01 using a two-tailed students t-test. (D) Simvastatin significantly impacts tumor growth in vivo. 2×10⁶ MDA-231 cells were injected subcutaneously into 8 week-old NOD-SCID mice. Fourteen days after implantation mice were paired by equal tumor volumes and randomized to either a Simvastatin (200 mg/kg/day) or Control (placebo) group (N=5 for each group). Tumor measurements were performed weekly using calipers. After 21 days of treatment, mice were sacrificed and tumors were extracted and weighed. Tumor volumes as a function of time (left) and tumor weights at day 21 (right) are presented. *denotes p<0.01, **denotes p<0.001 using a two-tailed students t-test.

FIG. 12. Mutant p53 regulates SREBP target genes in breast cancer cells: Venn diagram illustrating overlap between SREBP target genes and genes changed after mutant p53 knockdown. Significant gene expression changes (p<0.05) from genome-wide expression analysis of MDA-468 cells depleted of mutant p53 were queried against a comprehensive list of SREBP1 target genes (Reed et al., 2008). P-value was determined by the Chi-squared method. (B) Sterol biosynthesis genes regulated by mutant p53. MDA-468.shp53 cells were grown in 3D culture for 8 days in the presence or absence of DOX to knockdown mutant p53. qRT-PCR of three independent experiments of sterol biosynthesis genes not initially identified using IPA. Data presented as mean±st dev. *indicates p<0.05, ** indicates p<0.01 using a two-tailed t-test (C) Sterol biosynthesis genes regulated by mutant p53 in MDA-231 cells. MDA-231.shp53 cells were grown in 3D culture for 8 days in the presence or absence of DOX to knockdown mutant p53. qRT-PCR of three independent experiments of sterol biosynthesis genes. Data presented as mean±st dev. *indicates p<0.05 using a two-tailed t-test. (D) SREBP target genes, including fatty acid biosynthesis genes, regulated by mutant p53. MDA-468.shp53 cells were grown in 3D culture for 8 days in the presence or absence of DOX to knockdown mutant p53. qRT-PCR of three independent experiments of SREBP target genes. Data presented as mean±stdev. **indicates p<0.01 using a two-tailed t-test.

FIG. 13. Mutant p53 is correlated with higher expression of a subset of sterol biosynthesis genes in human breast cancer patient datasets, (Related to FIG. 6): Five human breast cancer patient datasets were analyzed to determine whether tumors bearing mutant p53 correlate with higher expression of sterol biosynthesis genes. Patients were stratified based on TP53 status (wild-type vs. mutant) and expression levels for sterol biosynthesis genes were analyzed. p-value represents the result of a one-sided t-test. See Table 1 for all genes. (A) Farnesyl Diphosphate Synthase (FDPS) exhibited higher expression levels in mutant p53 tumors compared to wild-type p53 tumors (p<0.05) in three out of five datasets. (B) Squalene Epoxidase (SQLE) exhibited higher expression levels in mutant p53 tumors compared to wild-type p53 tumors (p<0.05) in four out of five datasets. (C) 7-Dehydrocholesterol reductase (DHCR7) exhibited higher expression levels in mutant p53 tumors compared to wild-type p53 tumors (p<0.05) across all five datasets.

### SUMMARY OF THE INVENTION

Certain embodiments are directed to a method for determining if a subject having cancer, precancerous cells or a benign tumor should be treated with an inhibitor selected from the group comprising an inhibitor of one or more enzymes in the mevalonate pathway, an inhibitor of geranylgeranyl transferase or an inhibitor of farnesyl transferase, comprising: (i) obtaining a sample of the cancer cells, the precancerous cells or the benign tumor cells from the subject, ii) assaying the cells in the sample for the presence of a mutated p53 gene or a mutant form of p53 protein or a biologically active fragment thereof, and iii) if the cells have the mutated p53 gene or mutant form of the p53 protein, then determining that the subject should be treated with the inhibitor.

In the above method the cancer cells and the precancerous cells are obtained from a tumor or a biological sample from the subject such as tumor biopsy or a biological sample comprising urine, blood, cerebrospinal fluid, sputum, serum, stool or bone marrow. In an embodiment a DNA hybridization assay is used to detect the p53 gene in the sample. The cancer to be treated includes the cancer cells are selected from the group comprising lung cancer, digestive and gastrointestinal cancers, gastrointestinal stromal tumors, gastrointestinal carcinoid tumors, colon cancer, rectal cancer, anal cancer, bile duct cancer, small intestine cancer, and stomach (gastric) cancer, esophageal cancer, gall bladder cancer, liver cancer, pancreatic cancer, appendix cancer, breast cancer, ovarian cancer, renal cancer, cancer of the central nervous system, skin cancer, lymphomas, choriocarcinomas, head and neck cancers, osteogenic sarcomas, and blood cancers. In an embodiment the cancer is breast cancer that is hormone receptor-negative (ER−/PR−).

Another embodiment is a method for treating a subject having cancer, precancerous cells, or a benign tumor that has a mutated p53 gene or mutant p53 protein, by administering to the subject a therapeutically effective amount of an inhibitor of one or more enzymes in the mevalonate pathway, geranylgeranyl transferase, or farnesyl transferase. In an embodiment of the above methods theinhibitor is a statin selected from the group comprising lovastatin, simvastatin, pravastatin, fluvastatin, atorvastatin, and cerivastatin. In some embodiments the statin is lipophilic statin selected from the group comprising simvastatin, lovastatin, fluvastatin, cerevastatin and atrovastatin. In some embodiments the statin is a hydrophilic statin, selected from the group comprising rosuvastatin and pravastatin. In an embodiment the therapeutically effective amount of the statin is from about 0.1 mg/day to about 150 mg/day.

In the method of treatment, the inhibitor(s) is administered orally, by injection, parenterally, by inhalation spray, topically, rectally, nasally, buccally, vaginally or via an implanted reservoir. In another embodiment the inhibitor(s) is administered locally to the site of the cancer or benign tumor. In an embodiment the inhibitor of geranylgeranyl transferase is GGTI-2133, the inhibitor of farnesyl transferase is a member selected from the group comprising FTI-277, and the inhibitor of squalene synthase is YM-5360.1.

Some embodiments are directed to pharmaceutical formulations comprising one or more statins combined with a nonstatin inhibitor of an enzyme in the mevalonate pathway; or combined with one or more compounds selected from the group comprising an inhibitor of an enzyme in the inhibitor of geranylgeranyl transferase, and the inhibitor of farnesyl transferase. In another embodiment a formulation comprises one or more statins each of which is in an amount above 80 mg. In some embodiments the amount is between 80 and 150 mg, and in some it is between 150 and 250 mg, and in some it is between 250 and 350 mg, in some it is between 350 mg and 1 gram. The amount depends on the bioavailability, route of administration, the aggressiveness of the cancer, and whether the cancer is a tumor or circulating cancerous cells, for example.

Another embodiment is directed to a method for treating cancer, reducing precancerous lesions or benign tumors having a p53 mutation in the brain of a subject, comprising administering a therapeutically effective amount of a lipophilic inhibitor of one or more enzymes in the mevalonate pathway (such as one or more lipophilic statins), an inhibitor of geranylgeranyl transferase, an inhibitor of farnesyl transferase or an inhibitor of squalene synthase; or combinations thereof.

In another embodiment the method is for determining if cancer, precancerous lesions or benign tumors will respond to treatment with an inhibitor selected from the group comprising an inhibitor of one or more enzymes in the mevalonate pathway, an inhibitor of geranylgeranyl transferase, or an inhibitor of farnesyl transferase, comprising: (i) obtaining a sample of the cancer cells, the precancerous cells or the benign tumor cells from the subject, (ii) assaying the cells in the sample for the presence of a mutated p53 gene or a mutant form of p53 protein or a biologically active fragment thereof, and (iii) if the cells have the mutated p53 gene or mutant form of the p53 protein, then determining that the cancer will respond to treatment with the inhibitor.

In embodiments of the above methods, the enzyme is HMG-CoA synthase 1, and the inhibitor is 1233A; the enzyme is HMG-CoA reductase and the inhibitor is a statin; the enzyme is mevalonate decarboxylase and the inhibitor is 6-fluormevalonate; the enzyme is isopentyl diphosphate isomerase and the inhibitor is YM-16638; the enzyme is farnesyl diphosphate synthase and the inhibitor is a bisphosphanate that is selected from the group comprising; the enzyme is squalene synthase and the inhibitor is selected from the group comprising YM-53601, qualestatin-1 (zaragozic acid A), RPR-107393, ER-27856, BMS-188494, TAK-475; the enzyme is squalene epoxidase and the inhibitor is TU-2078 or NB-598; the enzyme is anosterol synthase and the inhibitor is Ro 28-8071 fumarate, or BIBB 515; the enzyme is lanosterol 14alpha demethylase and the inhibitor is that is selected from the group comprising SKF 104976, Azalanstat (RS-21607), and Miconazole; the enzyme is cholesterol C4-methyl oxidase and the inhibitor is 3-amino-1,2,4-triazole (ATZ); the enzyme is 7-dehydrocholesterol reductase and the inhibitor is BM 15766 or AY9944; the enzyme is desmosterol reductase and the inhibitor is brassicasterol; the enzyme is farnesyl transferase and the inhibitor is selected from the group comprising Tipifarnib (R115777), Lonafarnib (SCH66336), FTI-277, FTI-276, and FTI-2153; and the enzyme is geranylgeranyl transferase and the inhibitor is a selected from the group comprising GGTI-2133, GGTI-2418, GGTI-298, and GGTI-2154.

An embodiment is further directed to a method for preventing recurrence of cancer, precancerous lesions or a benign tumor having a mutated p53 gene or a mutant form of p53 protein or a biologically active fragment thereof, comprising administering a prophylactically effective amount of an inhibitor of one or more enzymes in the mevalonate pathway, geranylgeranyl transferase, or farnesyl transferase.

In subjects at high risk of developing a tumor/cancer comprising a p53 mutation, such as familial breast cancer, an embodiment is directed to preventing the tumors/cancer by administering one or more therapeutic agent inhibitors in a prophylactic amount.

### DEFINITIONS

As used herein, the terms “animal,” “patient,” or “subject” include mammals, e.g., humans, dogs, cows, horses, kangaroos, pigs, sheep, goats, cats, mice, rabbits, rats, and transgenic non-human animals. The preferred animal, patient, or subject is a human.

A “subject” or “patient” is a mammal, typically a human, but optionally a mammalian animal of veterinary importance, including but not limited to horses, cattle, sheep, dogs, and cats. A “therapeutic agent” is an inhibitor of one or more enzymes in the mevalonate pathway, and inhibitors of geranylgeranyl transferase, such as GGTI-2133, inhibitors of farnesyl transferase such as FTI-277, and inhibitors of squalene synthase such as YM-53601.

A “therapeutically effective amount” of a therapeutic agent is an amount that achieves the intended therapeutic effect of reducing cancerous cells, precancerous cells or benign tumor cells having a p53 protein or gene mutation in a subject. The full therapeutic effect does not necessarily occur by administration of one dose and may occur only after administration of a series of doses. Thus, a therapeutically effective amount may be administered in one or more administrations.

A “prophylactically effective amount” of a drug is an amount of a drug that, when administered to a subject, will have the intended prophylactic effect, e.g., preventing or delaying the onset (or reoccurrence) of the disease or symptoms, or reducing the likelihood of the onset (or reoccurrence) of the disease or symptoms. The full prophylactic effect does not necessarily occur by administration of one dose and may occur only after administration of a series of doses. Thus, a prophylactically effective amount may be administered in one or more administrations.

An “effective amount” of an agent is an amount that produces the desired effect.

“Treating” cancer in a patient refers to taking steps to obtain beneficial or desired results, including clinical results. For purposes of this invention, beneficial or desired clinical results include, but are not limited to alleviation or amelioration of one or more symptoms of the cancer; diminishing the extent of disease; delaying or slowing disease progression; amelioration and palliation or stabilization of the disease state.

The term “p53” as used herein refers to both p53 protein and the TP53 gene; “p53 mutations” refers to mutations in the p53 protein and p53 gene.

The term “TP53” as used herein refers to the gene encoding p53 protein.

The term “p53 protein” as used herein refers a tumor suppressor protein that in humans is encoded by the TP53 gene. p53 is crucial in multicellular organisms, where it regulates multiple cellular process such as cell cycle arrest, cell death, senescence, metabolic pathways and other outcomes thereby acting as a tumor suppressor that is involved in preventing cancer. p53 is also known as UniProt name: Cellular tumor antigen p53, Antigen NY-CO-13, Phosphoprotein p53, Transformation-related protein 53 (TRP53), Tumor suppressor p53.

The terms “polypeptide” and “protein” are used interchangeably as a generic term referring to native protein, fragments, peptides, or analogs of a polypeptide sequence. Hence, native protein, fragments, and analogs are species of the polypeptide genus.

The terms “treat” or “treatment” refer to both therapeutic treatment and prophylactic or preventative measures, wherein the object is to prevent or slow down (lessen) an undesired physiological change or disorder, such as the development, progression or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having cancer and those with benign tumors or precancerous lesions that have a mutant p53 gene.

The term “cancer” is intended to include any member of a class of diseases characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Examples of different types of cancer include, but are not limited to, lung cancer (e.g., non-small cell lung cancer); digestive and gastrointestinal cancers such as colorectal cancer, gastrointestinal stromal tumors, gastrointestinal carcinoid tumors, colon cancer, rectal cancer, anal cancer, bile duct cancer, small intestine cancer, and stomach (gastric) cancer; esophageal cancer; gallbladder cancer; liver cancer; pancreatic cancer; appendix cancer; breast cancer; ovarian cancer; renal cancer (e.g., renal cell carcinoma); cancer of the central nervous system; skin cancer; lymphomas; choriocarcinomas; head and neck cancers; osteogenic sarcomas; and blood cancers. As used herein, a “tumor” comprises one or more cancer cells or benign cells or precancerous cells.

The term “gene” includes the segment of DNA involved in producing a polypeptide chain. Specifically, a gene includes, without limitation, regions preceding and following the coding region, such as the promoter and 3′-untranslated region, respectively, as well as intervening sequences (introns) between individual coding segments (exons).

The term “nucleic acid” or “polynucleotide” includes deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, and mRNA encoded by a gene.

A “single nucleotide polymorphism” or “SNP” occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site, and occurs in at least 1% of the population.

The term “genotype” as used herein includes to the genetic composition of an organism, including, for example, whether a diploid organism is heterozygous or homozygous for one or more variant p53 alleles of interest.

The term “sample” as used herein includes any biological specimen obtained from a subject. Samples include, without limitation, whole blood, plasma, serum, red blood cells, white blood cells (e.g., peripheral blood mononuclear cells), saliva, urine, stool (i.e., feces), tears, nipple aspirate, lymph, fine needle aspirate, any other bodily fluid, a tissue sample (e.g., tumor tissue) such as a biopsy of a tumor, and cellular extracts thereof. In some embodiments, the sample is whole blood or a fractional component thereof such as plasma, serum, or a cell pellet. In certain embodiments, the sample is obtained by isolating circulating cells of a solid tumor from a whole blood cell pellet using any technique known in the art. As used herein, the term “circulating cancer cells” comprises cells that have either metastasized or micro metastasized from a solid tumor and includes circulating tumor cells, and cancer stem cells. In other embodiments, the sample is a formalin fixed paraffin embedded (FFPE) tumor tissue sample, e.g., from a solid tumor.

A nucleic acid sample can be obtained from a subject using routine methods. Such samples comprise any biological matter from which nucleic acid can be prepared. As non-limiting examples, suitable samples include whole blood, serum, plasma, saliva, cheek swab, urine, or other bodily fluid or tissue that contains nucleic acid. In one embodiment, the methods of the present invention are performed using whole blood or fractions thereof such as serum or plasma, which can be obtained readily by non-invasive means and used to prepare genomic DNA. In another embodiment, genotyping involves the amplification of a subject's nucleic acid using PCR. Use of PCR for the amplification of nucleic acids is well known in the art (see, e.g., Mullis et al., The Polymerase Chain Reaction, Birkhauser, Boston, (1994). Generally, protocols for the use of PCR in identifying mutations and polymorphisms in a gene of interest are described in Theophilus et al., “PCR Mutation Detection Protocols,” Humana Press (2002). Further protocols are provided in Innis et al., “PCR Applications: Protocols for Functional Genomics,” 1st Edition, Academic Press (1999). Applicable PCR amplification techniques are described in Ausubel et al., Current Protocols in Molecular Biology, John Wiley & Sons, Inc., New York (1999); Theophilus et al., “PCR Mutation Detection Protocols,” Humana Press (2002); and Innis et al., “PCR Applications: Protocols for Functional Genomics,” 1st Edition, Academic Press (1999). General nucleic acid hybridization methods are described in Anderson, “Nucleic Acid Hybridization,” BIOS Scientific Publishers (1999). Amplification or hybridization of a plurality of transcribed nucleic acid sequences (e.g., mRNA or cDNA) can also be performed using mRNA or cDNA sequences arranged in a microarray. Microarray methods are generally described in Hardiman, “Microarrays Methods and Applications: Nuts & Bolts,” DNA Press (2003) and Baldi et al., “DNA Microarrays and Gene Expression: From Experiments to Data Analysis and Modeling,” Cambridge University Press (2002).

Primer sequences and amplification protocols for evaluating p53 mutations are known to those in the art and have been published. For a list of primer sequences used to sequence p53, refer to: Reles et al. Correlation of p53 Mutations with Resistance to Platinum-based Chemotherapy and Shortened Survival in Ovarian Cancer. Clinical Cancer Research (2001).

Examples of TP53 mutations are described in, e.g., Soussi T. (2007) Cancer Cell 12(4):303-12; Cheung K. J. (2009) Br J. Haematol. 146(3):257-69; Pfeifer G. P. et al. (2009) Hum Genet. 125(5-6):493-506; Petitjean A. et al. (2007) Oncogene 26(15):2157-65.

### DETAILED DESCRIPTION

It has been discovered that cancer cells that have a p53 mutation can be contacted with an inhibitor of one or more enzymes in the mevalonate pathway or enzymes in certain pathways that are offshoots of the mevalonate pathway, to normalize the abnormal phenotype. In vivo results showed that simvastatin reduced tumor size after 21 days of treatment by about 40%. Certain embodiments of the present invention are directed to methods for determining if a subject with cancer or precancerous lesions or a benign tumor, will respond to treatment (i.e. if the patient and the cancer will respond to treatment) with an inhibitor selected from the group comprising an inhibitor of one or more enzymes in the mevalonate pathway, an inhibitor of geranylgeranyl transferase, or an inhibitor of farnesyl transferase, by (i) obtaining a sample of the cancer cells, precancerous cells or benign tumor cells from the subject, (ii) assaying the cells in the sample for the presence of a mutated p53 gene or a mutant form of p53 protein or a biologically active fragment thereof, and (iii) if the cells have the mutated p53 gene or mutant form of the p53 protein, then determining that the subject will respond to treatment with the inhibitor or combinations thereof. Other embodiments are directed to a method for treating a subject having cancer, precancerous cells, or having a benign tumor that has a mutated p53 gene or mutant p53 protein by administering a therapeutically effective amount of an inhibitor selected from the group comprising an inhibitor of one or more enzymes in the mevalonate pathway, an inhibitor of geranylgeranyl transferase, or an inhibitor of farnesyl transferase to the subject. Other embodiments are directed to methods for either reducing the number of precancerous cells that have a p53 mutation or reducing the number of benign tumor cells that have a p53 mutation in a patient by administering a therapeutically effective amount of one or more of the herein described inhibitors/therapeutic agents.

Statins are known to inhibit HMG-CoA reductase in the mevalonate pathway, therefore these agents can be administered therapeutically to treat cancer that has a p53 mutation. (p53 mutation herein generally refers to both a mutation in the TP53 gene or the expressed protein. Lipophilic statins can cross the blood brain barrier (BBB), so these are preferred for treating any brain cancer, precancerous lesion or benign tumor. (Vuletic et al. 2006), Lipophilic statins bypass the liver so that they are useful for non-liver cancer, etc. Hydrophilic statins are preferred for liver cancer, precancerous lesions or benign tumors since they are taken up by the liver. The experiments herein described contacted cells in 3D culture with lipophilic statins, simvastatin or mevastatin, at the following concentrations: 100 nM or 1 μM, which range approximates the clinically achievable serum concentrations in human patients following a 40-80 mg/day dose schedule (Dimitroulakos et al., 1999; Wong et al., 2002).

Any agent that inhibits an enzyme in the mevalonate pathway, or combinations of the agents, can be used to treat cancer or reduce the number of precancerous cells or benign tumor cells if they have a p53 gene or protein mutation. The results of the experiments described below also show that inhibition of geranylgeranyl transferase, a key offshoot of the mevalonate pathway, is also very important for mediating the effects of mutant p53. Therefore certain embodiments are directed to methods for treating cancer, precancerous lesions and benign tumors with inhibitors of geranylgeranyl transferase, such as GGTI-2133. Other enzyme inhibitors of farnesyl transferase such as FTI-277, and of squalene synthase such as YM-53601 are also within the scope of the present invention.

Other embodiments are directed to adjuvant therapies to prevent recurrence of cancer or precancerous cells or benign tumors that have a p53 gene or protein mutations, by administering a prophylactic amount of one of the herein described inhibitors. In some cases, even before the present discoveries, cancerous cells or tumors have been analyzed for the presence of p53 mutations. In an embodiment, subjects who have been treated for cancer or a precancerous lesion that had a p53 protein or gene mutation are treated to prevent recurrence of the cancer or lesion by administering a prophylactic amount of one of the herein described inhibitors.

### SUMMARY

p53 is a frequent target for mutation in mammalian tumors and previous studies have revealed that missense mutant p53 proteins can actively contribute to tumorigenesis. p53 mutations are usually thought to occur is 25-40% of breast cancers, but some studies report that two-thirds of all breast cancers display p53 mutations (Lai et al. (2004) Breast Cancer Res. Treat., 83: 57-66). Aberrant forms of human p53 are associated with poor prognosis, more aggressive tumors, metastasis, and short survival rates in multiple tumor types (Mitsudomi et al., Clin Cancer Res 2000 October; 6(10):4055-63; Koshland, Science (1993) 262:1953), (Petijean et al. 2007). The results described herein implicate the mevalonate pathway as a new therapeutic target for tumors bearing p53 mutations.

The results of experiments described herein show that:
Depletion of endogenous mutant p53 from breast cancer cells is sufficient to induce a phenotypic reversion in 3D culture from a cancerous morphology to a more normal hollow—lumen acinar morphology. Functional transactivation domains are necessary for mutant p53 to disrupt acinar morphogenesis. [Example 2]
Mutant p53 upregulates seventeen genes that encode enzymes in the mevalonate pathway. [Example 3].
The effects of mutant p53 on breast cancer morphology are mediated through the mevalonate pathway. HMG-CoA reductase inhibitors mimic the phenotypic effects of mutant p53 depletion in 3D culture thereby causing the cancer cells to revert to normal morphology or result in a more profound phenotypic effect (i.e. cell death).
The normalizing phenotypic effects following downregulation of mutant p53 can be recapitulated by inhibiting critical enzymes in the mevalonate pathway. This normalization can be reversed by supplementing breast cancer cells depleted of mutant p53 with two key intermediate metabolites produced by this pathway, specifically mevalonic acid (MVA) and mevalonic acid 5-phosphate (MVAP). Thus, flux through the mevalonate pathway is both necessary and sufficient for the phenotypic effects of mutant p53 on breast cancer morphogenesis in 3D culture. HMG-CoA reductase inhibitors mimic the phenotypic effects of mutant p53 depletion in breast cancer cells.
In vivo mouse data shows that treatment with simvastatin reduced tumor size after 21 days of treatment by about 40%. Example 5.
Not only HMG-CoA reductase, but several downstream enzymatic steps in the mevalonate pathway are involved in the ability of mutant p53 to prevent normal morphological behavior of breast cancer cells in 3D culture conditions.
Patient data shows that TP53 mutation correlates with high levels of sterol biosynthesis genes in human tumors [Example 4]. Interestingly, at least one clinical study investigating the effect of statins in breast cancer noted a subgroup-specific protective effect: specifically, a significantly decreased incidence of hormone receptor-negative (ER−/PR−) tumors was documented in patients takings statins, while no such effect was observed for hormone receptor-positive tumors (Kumar et al., 2008). Preclinical models, employing either breast cancer cell lines or mouse models of breast cancer, also support a more dramatic role for statins in ER−/PR− breast cancers (Campbell et al., 2006; Garwood et al., 2010). Further, many studies have shown that the majority of breast tumors that bear p53 mutations are also immunohistochemically classified as ER−/PR− (Han et al., 2011; Sorlie et al., 2001) For example, p53 is mutated in about 25-30% or ductal carcinoma in situ (DCIS) cases of breast cancer. These patients would y benefit from statin therapy./prophylaxis.
The experimental results herein show that a subset of the sterol biosynthesis genes are significantly higher in large cohorts of human breast tumors bearing mutant p53 which shows that the ability of mutant p53 to upregulate the sterol biosynthesis genes is not constrained to a single class of p53 mutations. Thus the present methods for treating cancer or reducing the number of precancerous cells or benign tumor cells with p53 mutations with the described inhibitors of sterol biosynthesis can be broadly used for any p53 mutation.

Breast cancer is thought to arise from mammary epithelial cells found in structures referred to as acini, which collectively form terminal ductal lobular units (TDLU). Each acinus consists of a single layer of polarized luminal epithelial cells surrounding a hollow-lumen (Allred et al., 2001; Bissell et al., 2002). Normal mammary epithelial cells, when grown in a laminin-rich extracellular matrix, form three-dimensional structures highly reminiscent of many aspects of acinar structures found in vivo (Debnath et al., 2003; Petersen et al., 1992), and the processes and pathways that govern and disrupt normal mammary epithelial development in this setting have been defined (Debnath et al., 2002; Muthuswamy et al., 2001; Wrobel et al., 2004; Zhan et al., 2008). Since one of the hallmarks of breast tumorigenesis is the disruption of mammary tissue architecture (Friedrich, 2003), three-dimensional (3D) culture conditions allow one to readily distinguish normal and tumorigenic tissue by morphological phenotype (Kenny et al., 007; Martin et al., 2008; Muthuswamy et al., 2001). Inhibition of key oncogenic signaling pathways are sufficient to phenotypically revert breast cancer cells grown in 3D culture (Beliveau et al., 2010; Bissell et al., 2005; Wang et al., 1998; Weaver et al., 1997). The list of known proteins, modulation of which is sufficient to induce a phenotypic reversion in tumorigenic breast cells grown in 3D culture, is set forth in Table 3. In an embodiment the therapeutic agents of the invention are administered together with one or more of the proteins in Table 3 to treat cancer, precancerous lesions or benign tumors.

The mevalonate pathway has recently been implicated in multiple aspects of tumorigenesis, including proliferation, survival, invasion and metastasis (Clendening et al., 2010; Dimitroulakos et al., 1999; Kidera et al., 2010; Koyuturk et al., 2007; Wejde et al., 1992). Competitive inhibitors of the rate-limiting enzyme in the mevalonate pathway, HMG-CoA reductase, collectively known as statins, have been reported to be cancer-protective for certain malignancies, including breast cancer (Blais et al., 2000; Cauley et al., 2003; Stein E A, 1993); however, there are an equal number of reports against the use of statins to treat breast cancer, for example REFS (Baigent et al., 2005; Browning and Martin, 2007). The statins have already been employed in multiple preclinical models of breast cancer (Kubatka et al., 2011; Shibata et al., 2004) and two reports have demonstrated a significant impact of Simvastatin treatment on growth of MDA-231 breast cancer xenografts in nude mice (Ghosh-Choudhury et al., 2010; Mori et al., 2009).

However, the fact that there are many contradictory reports can be attributed to the fact that until now, it was not known how to identify those types of cancer that would respond to treatment with a statin or other inhibitor to an enzyme in the mevalonate or a closely related pathway. The experiments described herein using breast cancer cells, show that p53 mutations result in enhanced mevalonate production, and that blocking this enzyme or an enzyme in certain closely related pathways in a breast cancer cell having a p53 mutant inhibits cancer cell growth and normalizes the morphology, or even kills the cancer cell.

Enzymes in the mevalonate pathway that can be used in the methods of the present invention include:

Acetylacetyl-CoA Transferase

HMG-CoA Synthase

HMG-CoA reductase

Mevalonate Kinase

Phosphomevalonate Kinase

Mevalonate Decarboxylase

Isopentyl Diphosphate Isomerase

Farnesyl Diphosphate Synthase

Farnesyl Transferase

Geranylgeranyl Transferase

Squalene Synthase

Squalene Epoxidase

Lanosterol Synthase

Lanosterol 14alpha Demethylase

Sterol C14 Reductase

Cholesterol C4-Methyl Oxidase

NAD(P)H Steroid Dehydrogenase

7-Dehydrocholesterol Reductase

Desmosterol Reductase

Other enzymes in closely related pathways that can be targeted to inhibit cancer cell growth include: farnesyl transferase, and geranylgeranyl transferase. The enzymes and certain inhibitors (preflixed with a lower case letter) in the mevalonate pathway include the following:

1. Acetylacetyl-CoA transferase

2. HMG-CoA synthase 1
a. 1233A

3. HMG-CoA reductase
a. Statins (Simvastatin, Mevastatin, Fluvastatin, Atorvastatin, Cerivastatin, Lovastatin)

4. Mevalonate Kinase

5. Phosphomevalonate Kinase

6. Mevalonate Decarboxylase
a. 6-fluoromevalonate

7. Isopentyl Diphosphate Isomerase
a. YM-16638

8. Farnesyl Diphosphate Synthase
a. Bisphosphanates (Risedronate, Zoledronate, Ibandronate, Alendronate, Pamidronate, Neridronate, Olpadronate, Etidronate, Clodronate, Tiludronate)

9. Squalene Synthase
a. YM-53601
b. Squalestatin-1 (zaragozic acid A)
c. RPR-107393
d. ER-27856
e. BMS-188494
f. TAK-475

10. Squalene Epoxidase
a. TU-2078
b. NB-598

11. Lanosterol Synthase
a. Ro 28-8071 fumarate
b. BIBB 515

12. Lanosterol 14alpha Demethylase
a. SKF 104976
b. Azalanstat (RS-21607)
c. Miconazole

13. Sterol C14 Reductase

14. Cholesterol C4-Methyl Oxidase
a. 3-amino-1,2,4-triazole (ATZ)

15. NAD(P)H Steroid Dehydrogenase

16. 7-Dehydrocholesterol Reductase
a. BM 15766
b. AY9944

17. Desmosterol Reductase
a. Brassicasterol

Inhibitors to Mevalonate Pathway Related Enzymes

1. Farnesyl Transferase
a. Tipifarnib (R115777)
b. Lonafarnib (SCH66336)
c. FTI-277
d. FTI-276
e. FTI-2153

2. Geranylgeranyl Transferase
a. GGTI-2133
b. GGTI-2418
c. GGTI-298
d. GGTI-2154
   Identifying Cancer Cells with p53 Mutations

The subgroup of breast cancer patients displaying p53 mutations generally respond poorly to therapy and exhibit rapidly growing tumors and shorter median survival (Lai et al., supra; Reed (1996) J. Clin. Invest., 97:2403-2404). Aberrant forms of human p53 are associated with poor prognosis, more aggressive tumors, metastasis, and short survival rates (Mitsudomi et al., Clin Cancer Res 2000 October; 6(10):4055-63; Koshland, Science (1993) 262:1953). The Gene ID for TP53 is 7157.

Alterations of a wild-type p53 gene according to the present invention encompass all forms of mutations such as insertions, inversions, deletions, and/or point mutations. Somatic mutations are those which occur only in certain tissues, e.g., in the tumor tissue, and are not inherited in the germ line. If only a single allele is somatically mutated, an early neoplastic state is indicated. However, if both alleles are mutated then a late neoplastic state is indicated. Germ line mutations can be found in any of a body's tissues. Patients who have Li-Fraumeni inherit germ-line mutations in TP53, however germ line TP53 mutations are rare. In an embodiment Li-Fraumeni patients can be treated by administering a therapeutic agent that inhibits one or more enzymes in the mevalonate pathway to treat or prevent cancer that has a p53 mutation. The finding of p53 mutations in a benign tumor is also a condition that can be treated prophylactically.

Cancer (and precancerous lesions) that can be treated with the methods of the present invention include any tumor or cancerous cell that has a p53 mutation. Such cancers include breast cancer, neuroblastoma, gastrointestinal carcinoma such as rectum carcinoma, colon carcinoma, familial adenomatous polyposis carcinoma and hereditary non-polyposis colorectal cancer, esophageal carcinoma, labial carcinoma, larygial carcinoma, hypopharyngial carcinoma, tongue carcinoma, salivary gland carcinoma, gastric carcinoma, medullary thyroid carcinoma, papillary thyroid carcinoma, renal carcinoma, kidney parenchymal carcinoma, ovarian carcinoma, cervical carcinoma, uterine corpus carcinoma, endometrium carcinoma, choriocarcinoma, pancreatic carcinoma, prostate carcinoma, testis carcinoma, urinary carcinoma, melanoma, brain tumors such as glioblastoma, astrocytoma, meningioma, medulloblastoma and peripheral neuroectodermal tumors, Hodgkin's lymphoma, non-Hodgkin's lymphoma, Burkitt's lymphoma, acute lymphocytic leukemia (ALL), chronic lymphocytic leukemia (CLL), acute myelologenous leukemia (AML), chronic myelologenous leukemia (CML), adult T-cell leukemia/lymphoma, hepatocellular carcinoma, gallbladder carcinoma, bronchial carcinoma, small cell lung carcinoma, non-small cell lung carcinoma, multiple myeloma, basal cell carcinoma, teratoma, retinoblastoma, choroidal melanoma, seminoma, rhabdomyosarcoma, craniopharyngioma, osteosarcoma, chondrosarcoma, myosarcoma, liposarcoma, fibrosarcoma, Ewing's sarcoma and plasmocytoma. Particular tumors include those of the brain, liver, kidney, bladder, breast, gastric, ovarian, colorectal, prostate, pancreatic, lung, vulval, thyroid, colorectal, oesophageal, sarcomas, glioblastomas, head and neck, leukemias and lymphoid malignancies.

Mutant p53 genes or gene products can be detected in tumor samples or, in some types of cancer, in biological samples such as urine, stool, sputum or serum. For example, TP53 mutations can often be detected in urine for bladder cancer and prostate cancer, sputum for lung cancer, or stool for colorectal cancer. Serum has mostly been tested in the context of colorectal cancer, however this should work for any tumor type that sheds cancer cells into the blood. Cancer cells are found in blood and serum for cancers such as lymphoma or leukemia. The same techniques discussed above for detection of mutant p53 genes or gene products in tumor samples can be applied to other body samples. Cancer cells are sloughed off from tumors and appear in such body samples.

A p53 (TP53) gene mutation in a sample can be identified using any method known in the art. One of the most commonly used methods to “identify” p53 mutants is by utilizing immunohistochemistry (IHC) on tumor sections stained with a p53 antibody. Positive staining with an antibody against p53 is often used as a surrogate for sequencing the gene itself. Some have proposed combining sequencing and IHC, since p53 mutants that are highly expressed tend to be more oncogenic

In one assay nucleic acid from the sample is contacted with a nucleic acid probe that is capable of specifically hybridizing to nucleic acid encoding a mutated p53 protein, or fragment thereof incorporating a mutation, and detecting the hybridization. In a particular embodiment the probe is detectably labeled such as with a radioisotope, a fluorescent agent (rhodamine, fluorescene) or a chromogenic agent. In a particular embodiment the probe is an antisense oligomer. The probe may be from about 8 nucleotides to about 100 nucleotides, or about 10 to about 75, or about 15 to about 50, or about 20 to about 30. Kits for identifying p53 mutations in a sample are available that include an oligonucleotide that specifically hybridizes to or adjacent to a site of mutation in the p53 gene. The p53 Amplichip™ developed by Roche is a good example of this technology; http://www.ncbi.nlm.nih.gov/pmc/articles/PMC2691672/?tool=pubmed.

Using gene expression signatures, it has been shown that most p53 mutations cluster in the basal-like subgroup of breast cancers, which has the poorest prognosis and is notoriously difficult to treat (Perou et al., 2000). Using a combination of expression signatures and data from over 40,000 compounds screened in the NCl-60 cell lines, Mori et al. predicted three FDA-approved drugs to be most effective for treating basal-like breast cancers, two of which, Simvastatin and Lovastatin, are inhibitors of HMG-CoA reductase (Mori et al., 2009). Embodiments of the present invention provide a means for stratifying breast cancer patients based on their p53 mutational status to identify patients who will respond to treatment with a statin or other inhibitor of one or more enzymes in the mevalonate pathway.

Not all p53 mutations are equivalent. Genetic alterations in p53 are often grouped into two classes based on the type of mutant p53 that they produce (Brosh and Rotter, 2009). Contact mutants, exemplified by p53-R273H, involve mutation of residues that are directly involved in protein-DNA contacts. Conformational mutants, typified by p53-R175H, result in conformational distortions in the p53 protein. The experimental results herein show that a subset of the sterol biosynthesis genes are significantly higher in large cohorts of human breast tumors bearing mutant p53 which shows that the ability of mutant p53 to upregulate the sterol biosynthesis genes is not constrained to a single class of p53 mutations. Thus the present methods for treating cancer, precancerous lesions or preventing benign tumors with p53 mutations from becoming cancerous can be broadly used for any p53 mutation.

A mutation in the p53 gene in a sample can be detected by amplifying nucleic acid corresponding to the p53 gene obtained from the sample, or a biologically active fragment, and comparing the electrophoretic mobility of the amplified nucleic acid to the electrophoretic mobility of corresponding wild-type p53 gene or fragment thereof. A difference in the mobility indicates the presence of a mutation in the amplified nucleic acid sequence. Electrophoretic mobility may be determined on polyacrylamide gel. Alternatively, an amplified p53 gene or fragment nucleic acid may be analyzed for detection of mutations using Enzymatic Mutation Detection (EMD) (Del Tito et al, Clinical Chemistry 44:731-739, 1998). EMD uses the bacteriophage resolvase T₄ endonuclease VII, which scans along double-stranded DNA until it detects and cleaves structural distortions caused by base pair mismatches resulting from point mutations, insertions and deletions. Detection of two short fragments formed by resolvase cleavage, for example by gel eletrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T₄ endonuclease VII as demonstrated in U.S. Pat. No. 5,869,245.

In order to detect the mutation of the wild-type p53 gene, a sample or biopsy of the tumor or a sample comprising cancer cells or precancerous cells (such as blood, serum, CSF, stool, urine or sputum) is obtained by methods well known in the art and appropriate for the particular type and location of the tumor. For instance, samples of breast cancer lesions may be obtained by resection, or fine needle aspiration. Means for enriching a tissue preparation for tumor cells are known in the art. For example, the tissue may be isolated from paraffin or cryostat sections. Cancer cells may also be separated from normal cells by flow cytometry or laser capture microdissection. These as well as other techniques for separating tumor from normal cells are well known in the art. If the tumor tissue is highly contaminated with normal cells, detection of mutations is more difficult.

Detection of point mutations may be accomplished by molecular cloning of the p53 allele (or alleles) and sequencing that allele(s) using techniques well known in the art. Alternatively, the polymerase chain reaction (PCR) can be used to amplify gene sequences directly from a genomic DNA preparation from the tumor tissue. The DNA sequence of the amplified sequences can then be determined and mutations identified. The polymerase chain reaction is the preferred method and it is well known in the art and described in Saiki et al., Science 239:487, 1988; U.S. Pat. Nos. 4,683,203; and 4,683,195.

The ligase chain reaction, which is known in the art, can also be used to amplify p53 sequences. See Wu et al., Genomics, Vol. 4, pp. 560-569 (1989). In addition, a technique known as allele specific PCR can be used. (See Ruano and Kidd, Nucleic Acids Research, Vol. 17, p. 8392, 1989.) According to this technique, primers are used which hybridize at their 3′ ends to a particular p53 mutation. If the particular p53 mutation is not present, an amplification product is not observed. Amplification Refractory Mutation System (ARMS) can also be used as disclosed in European Patent Application Publication No. 0332435 and in Newton et al., Nucleic Acids Research, Vol. 17, p. 7, 1989. Insertions and deletions of genes can also be detected by cloning, sequencing and amplification. In addition, restriction fragment length polymorphism, (RFLP) probes for the gene or surrounding marker genes can be used to score alteration of an allele or an insertion in a polymorphic fragment. Single stranded conformation polymorphism (SSCP) analysis can also be used to detect base change variants of an allele. (Orita et al., Proc. Natl. Acad. Sci. USA Vol. 86, pp. 2766-2770, 1989, and Genomics, Vol. 5, pp. 874-879, 1989.) Other techniques for detecting insertions and deletions as are known in the art can be used.

Mismatches, according to the present invention are hybridized nucleic acid duplexes which are not 100% complementary. The lack of total complementarity may be due to deletions, insertions, inversions, substitutions or frameshift mutations. Mismatch detection can be used to detect point mutations in the gene or its mRNA product. While these techniques are less sensitive than sequencing, they are simpler to perform on a large number of tumor samples. An example of a mismatch cleavage technique is the RNase protection method, which is described in detail in Winter et al., Proc. Natl. Acad. Sci. USA, Vol. 82, p. 7575, 1985 and Meyers et al., Science, Vol. 230, p. 1242, 1985. A labeled riboprobe which is complementary to the human wild-type p53 gene coding sequence can also be used. The riboprobe and either mRNA or DNA isolated from the tumor tissue are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the p53 mRNA or gene. If the riboprobe comprises only a segment of the p53 mRNA or gene it will be desirable to use a number of these probes to screen the whole mRNA sequence for mismatches.

In a similar manner, DNA probes can be used to detect mismatches, through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the cellular mRNA or DNA which might contain a mutation can be amplified using PCR before hybridization. Changes in DNA of the p53 gene can also be detected using Southern hybridization, especially if the changes are gross rearrangements, such as deletions and insertions.

DNA sequences of the p53 gene which have been amplified by use of polymerase chain reaction may also be screened using allele-specific probes. These probes include nucleic acid oligomers, each of which contains a region of the p53 gene sequence harboring a known mutation. For example, one oligomer may be about 30 nucleotides in length, corresponding to a portion of the p53 gene sequence. By use of a battery of such allele-specific probes, PCR amplification products can be screened to identify the presence of a previously identified mutation in the p53 gene. Hybridization of allele-specific probes with amplified p53 sequences can be performed, for example, on a nylon filter. Hybridization to a particular probe under stringent hybridization conditions indicates the presence of the same mutation in the tumor tissue as in the allele-specific probe. This is used with the p53 Amplichip described above.

Alteration of wild-type p53 genes can also be detected by screening for alteration of wild-type p53 protein. For example, monoclonal antibodies immunoreactive with p53 can be used to screen a tissue. As mentioned above, one of the common ways to “detect” p53 mutations is to see strong p53 immunostaining in tissue sections (these are not mutant p53 specific antibodies, but simply take advantage of the fact that most mutant p53 proteins are more stable (and thus more abundant) than wild-type p53. Antibodies specific for products of mutant alleles could also be used to detect mutant p53 gene product. Such immunological assays can be done in any convenient format known in the art. These include Western blots, immunohistochemical assays and ELISA assays. Any means for detecting an altered p53 protein or p53 mRNA can be used to detect alteration of wild-type p53 genes or the expression product of the gene. Point mutations may be detected by amplifying and sequencing the mRNA or via molecular cloning of cDNA made from the mRNA (or by sequencing genomic DNA). The sequence of the cloned cDNA can be determined using DNA sequencing techniques which are well known in the art. The cDNA can also be sequenced via the polymerase chain reaction (PCR).

In summary, it has been discovered that mutant p53 can disrupt mammary acinar morphology and that downregulation of mutant p53 in malignant breast cancer cells is sufficient to revert these cells to a normal phenotype. Mutant p53 is recruited to the promoters of many sterol biosynthesis genes leading to their upregulation. Tumors bearing p53 mutations may evolve to become highly reliant on metabolic flux through the mevalonate pathway, making them particularly sensitive to inhibition of this pathway. At a clinical level, inhibition of the mevalonate pathway, either alone or in combination with other therapies, offers a novel, safe and much needed therapeutic option for tumors bearing mutant p53.

### Administration of Therapeutic Agents

A “therapeutic agent” is an inhibitor of one or more enzymes in the mevalonate pathway, and inhibitors of geranylgeranyl transferase, such as GGTI-2133 and inhibitors of farnesyl transferase such as FTI-277. The therapeutically effective amount of a therapeutic agent depends upon a number of factors within the ken of the ordinarily skilled physician, veterinarian, or researcher and will vary depending inter alia on the subject, the activity and bioavailability of the specific agent (s) employed, the age, body weight, general health, gender, and diet of the subject, the time of administration, the route of administration, the rate of excretion, any drug combination, and the degree of expression or activity to be modulated. Contributing factors further include the type, location, aggressiveness and size of cancer, precancerous lesion or benign tumor. Some highly aggressive tumors may require higher therapeutic amounts, for example. The full therapeutic effect does not necessarily occur by administration of one dose and may occur only after administration of a series of doses. Thus, a therapeutically effective amount may be administered in one or more administrations, on the same day or on different days.

All statins block the same enzyme HMGCoA reductase and they have same binding site and mechanism of action. However they have different bioavailability and tissue specificity. In an embodiment, formulations of statins for treating brain cancer or reducing precancerous lesions or benign tumors in the brain or central nervous system comprise one or more lipophilic statins in a therapeutically effective amount.

Simvastatin has been approved by the FDA for up to 80 mg/day, which corresponds to a serum level of about 100 nanomolar to 1 micromolar. In the in vivo experiments described herein using mice, simvastatin was administered at a dose of 200 mg/kg/day, which corresponds to about 100 micromolar serum levels. However, humans can tolerate higher amounts than 80 mg/day, although adverse side effects can occur. Given the lethality of cancer and the high risk of precancerous lesions developing into cancer, the adverse side effects of administering higher than the FDA approved amount of 80 mg/day simvastatin (or other statin) is outweighed by the potential benefits. Therefore in certain embodiments the therapeutically effective amount a statin (or other therapeutic agent) falls within the FDA-approved use, for example to treat a nonaggressive form of cancer, for a precancerous lesion or benign tumor, or for long term administration or prophylactic use. In other embodiments the therapeutically effective amount is higher than the FDA-approved amount, for example for treating a highly aggressive cancer, or where the agent is administered directly to the tumor, or for a non-prolonged period of time.

Certain embodiments are directed to formulations comprising a therapeutically effective amount of one or more statins combined with one or more compounds selected from the group comprising an inhibitor of an enzyme in the mevalonate pathway, an inhibitor of geranylgeranyl transferase, such as GGTI-2133, an inhibitor of farnesyl transferase such as FTI-277, and an inhibitor of squalene synthase such as YM-53601.

Therapeutic agents may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. In some embodiments a slow release preparation comprising the therapeutic agents is administered. The therapeutic agents can be administered as a single treatment or in a series of treatments that continue as needed and for a duration of time that causes one or more symptoms of the cancer to be reduced or ameliorated, or that achieves another desired effect.

The dose(s) vary, for example, depending upon the identity, size, and condition of the subject, further depending upon the route by which the composition is to be administered and the desired effect. Appropriate doses of a therapeutic agent depend upon the potency with respect to the expression or activity to be modulated. The therapeutic agents can be administered to an animal (e.g., a human) at a relatively low dose at first, with the dose subsequently increased until an appropriate response is obtained.

A suitable subject is an individual or animal that has cancer, a precancerous lesion or has a benign tumor that has a p53 mutation. Administration of a therapeutic agent “in combination with” includes parallel administration of two agents to the patient over a period of time, co-administration (in which the agents are administered at approximately the same time, e.g., within about a few minutes to a few hours of one another), and co-formulation (in which the agents are combined or compounded into a single dosage form suitable for administration).

### Pharmaceutical Formulations

The therapeutic agents may be present in the pharmaceutical compositions in the form of salts of pharmaceutically acceptable acids or in the form of bases. The therapeutic agents may be present in amorphous form or in crystalline forms, including hydrates and solvates. Preferably, the pharmaceutical compositions comprise a therapeutically effective amount.

Pharmaceutically acceptable salts of the therapeutic agents described herein include those salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts. Other acids, such as oxalic, while not in themselves pharmaceutically acceptable, may be employed in the preparation of salts useful as intermediates in obtaining pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the therapeutic agents disclosed herein. Water or oil-soluble or dispersible products may be obtained by such quaternization.

The therapeutic agents of the present invention are also meant to include all stereochemical forms of the therapeutic agents (i.e., the R and S configurations for each asymmetric center). Therefore, single enantiomers, racemic mixtures, and diastereomers of the therapeutic agents are within the scope of the invention. Also within the scope of the invention are steric isomers and positional isomers of the therapeutic agents. The therapeutic agents of the present invention are also meant to include compounds which differ only in the presence of one or more isotopically enriched atoms. For example, therapeutic agents in which one or more hydrogens are replaced by deuterium or tritium, or the replacement of one or more carbons by ¹³C- or ¹⁴C-enriched carbon are within the scope of this invention.

In a preferred embodiment, the therapeutic agents of the present invention are administered in a pharmaceutical composition that includes a pharmaceutically acceptable carrier, adjuvant, or vehicle. The term “pharmaceutically acceptable carrier, adjuvant, or vehicle” refers to a non-toxic carrier, adjuvant, or vehicle that does not destroy or significantly diminish the pharmacological activity of the therapeutic agent with which it is formulated. Pharmaceutically acceptable carriers, adjuvants or vehicles that may be used in the compositions of this invention encompass any of the standard pharmaceutically accepted liquid carriers, such as a phosphate-buffered saline solution, water, as well as emulsions such as an oil/water emulsion or a triglyceride emulsion. Solid carriers may include excipients such as starch, milk, sugar, certain types of clay, stearic acid, talc, gums, glycols, or other known excipients. Carriers may also include flavor and color additives or other ingredients. The formulations of the combination of the present invention may be prepared by methods well-known in the pharmaceutical arts and described herein. Exemplary acceptable pharmaceutical carriers have been discussed above. An additional carrier, Cremophor™, may be useful, as it is a common vehicle for Taxol.

The pharmaceutical compositions of the present invention are preferably administered orally, preferably as solid compositions. However, the pharmaceutical compositions may be administered parenterally, by inhalation spray, topically, rectally, nasally, buccally, vaginally or via an implanted reservoir. Sterile injectable forms of the pharmaceutical compositions may be aqueous or oleaginous suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The pharmaceutical compositions employed in the present invention may be orally administered in any orally acceptable dosage form, including, but not limited to, solid forms such as capsules and tablets. In the case of tablets for oral use, carriers commonly used include microcrystalline cellulose, lactose and corn starch. Lubricating agents, such as magnesium stearate, are also typically added. When aqueous suspensions are required for oral use, the active ingredient may be combined with emulsifying and suspending agents. If desired, certain sweetening, flavoring or coloring agents may also be added.

The pharmaceutical compositions employed in the present invention may also be administered by nasal aerosol or inhalation. Such pharmaceutical compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Should topical administration be desired, it can be accomplished using any method commonly known to those skilled in the art and includes but is not limited to incorporation of the pharmaceutical composition into creams, ointments, or transdermal patches.

The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier. The passage of the agent can also be facilitated by its covalent coupling to a peptide vector capable of transporting the agent through the blood-brain barrier. Peptide transport vectors known as blood-brain barrier permeabilizer compounds are disclosed in U.S. Pat. No. 5,268,164. Site specific macromolecules with lipophilic characteristics useful for delivery to the brain are disclosed in U.S. Pat. No. 6,005,004.

Examples of routes of administration comprise parenteral, e.g., intravenous, intradermal, subcutaneous, inhalation, transdermal (topical), transmucosal, and rectal administration; or oral. Solutions or suspensions used for parenteral, intradermal, or subcutaneous application can comprise the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injection comprise sterile aqueous solutions (where water soluble) or dispersions and sterile powders for the extemporaneous preparation of sterile injectable solutions or dispersions. For intravenous administration, suitable carriers comprise physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included in the composition, for example, sugars, polyalcohols such as manitol, sorbitol, or sodium chloride. Prolonged absorption of an injectable composition can be achieved by including in the composition an agent that delays absorption, for example, aluminum monostearate or gelatin.

Sterile injectable solutions can be prepared by incorporating the active compound in the specified amount in an appropriate solvent with one or a combination of ingredients enumerated above, as needed, followed by filtered sterilization. Generally, dispersions are prepared by incorporating the active compound into a sterile vehicle that contains a basic dispersion medium and other ingredients selected from those enumerated above or others known in the art. In the case of sterile powders for the preparation of sterile injectable solutions, the methods of preparation comprise vacuum drying and freeze-drying which yields a powder of the active ingredient plus any additional desired ingredient from a previously sterile-filtered solution thereof.

Oral compositions generally comprise an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be comprised as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and comprise, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

### EXAMPLES

### Example 1

### Experimental Procedures

Plasmids, siRNA, Antibodies and Reagents

pLNCX-Flag-p53-R175H, -G245S, -R248Q, -R248W, -R273H and -L22Q/W23S/W53Q/F54S-R175H, -G245S, -R248Q, -R248W, -R273H were generated from pLNCX-Flag-p53-WT using the Stratagene QuikChange Site-Directed Mutagenesis kit according to the manufacturer's instructions. Mutagenesis primer sequences are provided in Table 2. pcDNA3.1-Myc-mSREBP-1a, -1c and -2 encode the mature forms of the SREBP transcription factors (Datta and Osborne, 2005). All constructs were verified by sequencing. p53 shRNA (2120) in STGM (tet-on) (Brekman et al., 2011) were used to establish cells with stable, inducible p53 knockdown. For transient knockdown experiments, siRNAs targeting SREBP1 (s129) or SREBP2 (s27) were purchased from Invitrogen. All-Stars (Control) and p53 siRNA were purchased from Qiagen.

p53 was detected using mAb 1801, DO-1 or 240. Anti-Actin (A2066), anti-Flag (F3165) and control IgG (I5381) antibodies were purchased from Sigma. Anti-Myc (sc-40) antibody was purchased from Santa Cruz. Anti-SREBP2 (1D2) is a monoclonal antibody raised against human SREBP-2 (hybridoma obtained from ATCC catalogue #CRL2545). Anti-SREBP2 (ab30682) antibody was purchased from Abcam. Alexa Fluor 594-Phalloidin (A12381) was purchased from Invitrogen.

Simvastatin (#10010344) and YM-53601 (#18113) were purchased from Cayman Chemicals. The following drugs were purchased from Sigma Aldrich: ALLN (A6185), Doxycycline (D9891), Simvastatin (S6196), Mevastatin (M2537), FTI-277 (F9803), GGTI-2133 (G5294), DL-Mevalolactone (M4667), DL-Mevalonic Acid 5-Phosphate (79849), Geranylgeranyl Pyrophosphate (#G6025) and Farnesyl Pyrophosphate (#6892). Fatostatin was synthesized by the Medicinal Chemistry Core Facility at the Sanford-Burnham Medical Research Institute as previously described (Kamisuki et al., 2009).

### Cell Lines and Generation of Stable Cell Lines

MDA-468, MDA-231, HEK 293 and Phoenix cells were maintained in DMEM+10% FBS. MCF10A cells were maintained in DMEM/F12 supplemented with 5% horse serum, 10 μg/ml Insulin, 0.5 μg/ml Hydrocortisone and 20 ng/ml Epidermal Growth Factor (EGF). All cells were maintained at 37° C. in 5% CO₂.

To generate stable cell lines with inducible shRNA, constructs were introduced into MDA-231 or MDA-468 cells by the retroviral mediated gene transfer method. Briefly, Phoenix packaging cells were transfected by the calcium phosphate method with either an rtTA plasmid or a vector expressing p53 shRNA or no shRNA. The generated viruses were harvested and MDA-231 or MDA-468 cells were co-infected with the rtTA and one of the vectors. After selection with puromycin (vector with shRNA) and hygromycin (rtTA), clonal cell lines were generated by the limited dilution method. Clonal cell lines were selected based on the level of p53 knockdown. Experiments shown were carried out on clonal cell lines or stable pools (MDA-468.shp53 pool, MDA-468.shp53 clone 1F5 and MDA-231.shp53 clone 1D10). To induce shRNA expression, cells were treated with 8 μg/ml doxycycline (DOX) for time periods indicated in the figure legends.

To generate stable, mutant p53 expressing cells, MCF10A cells were infected with pLNCX-Flag-p53-R175H, -G245S, -R248Q, -R248W, or -R273H, or a transactivation-deficient version of each mutant (mTAD): pLNCX-Flag-p53-22/23/53/54-R175H, -G245S, -R248Q, -R248W, -R273H and selected in G418 to yield stable pools.

To generate shRNA-resistant mutant p53 expressing cells, MDA-231.shp53 (Clone 1D10) cells were infected with pLNCX or pLNCX-Flag-p53-R273H which lacks the target site for the p53 shRNA, found in the 3′ UTR of the p53 mRNA. MDA-468.shp53 (Clone 1F5) cells were likewise infected with pLNCX, pLNCX-Flag-p53-R273H or pLNCX-Flag-p53-22/23/53/54-R273H (p53-R273H-mTAD) to generate shRNA-resistant mutant p53 expressing cells, either containing functional or non-functional transactivation domains, respectively. These cell lines were selected in G418 to generate stable pools.

### Three Dimensional (3D) Culture

Three-dimensional culture was carried out as previously described (Debnath et al., 2003). Briefly, 8-well chamber slides were lined with 50 μl growth factor reduced Matrigel (BD Biosciences). Cells were then seeded at a density of 5,000 cells/well in Assay Medium (DMEM/F12+2% Horse Serum+10 μg/ml Insulin+0.5 μg/ml Hydrocortisone [+5 ng/ml EGF for MCF10A cultures]) containing 2% Matrigel. Cells were refed with Assay Medium containing 2% Matrigel every 4 days. For RNA/protein analysis from 3D cultures, 35 mm plates were lined with 500 μl Matrigel and cells were seeded at a density of 225,000 cells/plate in Assay Medium+2% Matrigel. Cells were harvested using Cell Recovery Solution (BD Biosciences) according to the manufacturer's instructions.

### Immunostaining and Microscopy

Cells were fixed using 2% formaldehyde at room temperature for at least 30 min. Cells were permeabilized for 10 min at 4° C. with 0.5% Triton X-100 and subsequently blocked for 1 hr at room temperature with PBS+0.1% Tween-20+0.1% BSA+10% goat serum. Primary antibodies were incubated with the cultures for 1-2 hr at room temperature, followed by washing, and addition of fluorescently-conjugated secondary antibodies for 40 min at room temperature. Nuclei were counterstained with DRAQ5 (Cell Signaling #4084) or Propidium Iodide (Sigma #P4170). Confocal microscopy was conducted using an Olympus IX81 confocal microscope and analyzed using Fluoview software.

### Microarray and Data Analysis

RNA was isolated from three independent experiments of MDA-468.shp53 cells cultured under 3D conditions for 8 days in the presence or absence of DOX, reversed transcribed and hybridized to an Affymetrix GeneChip expression array. Data was processed using the Robust Multichip Average (RMA) algorithm to give expression signals and paired t-test was applied for each probe. Probes with 1% significance were selected for Ingenuity Pathway Analysis.

For Gene Ontology (GO) analysis, probe sets with the Detection Above Background (DABG) p-value <0.05 in at least one sample were used. Gene expression was calculated based on the mean value of all the probe sets for a gene. Gene expression changes were estimated by comparing cells grown in the presence of DOX versus those grown in the absence of DOX, and the three sample sets were analyzed separately. The GO annotation of genes was based on the NCBI Gene database. For each GO term, two p-values were calculated using Fisher's exact test by examining whether a significant fraction of genes associated with the GO term were up-regulated or down-regulated beyond the 1*standard deviation of all genes based on log 2(ratio). The smaller p-value was used to represent the trend of regulation. GEO Accession Number: GSE31812.

### Quantitative RT-PCR

RNA was isolated from cells using the Qiagen RNeasy Mini Kit according to the manufacturer's instructions. Complementary DNA was transcribed using Qiagen Quantitect reverse transcription kit. Real-time PCR was carried out on an ABI StepOne Plus using SYBR green dye. Transcript levels were assayed in triplicate and normalized to RPL32 mRNA expression. Relative levels were calculated using the Comparative-Ct Method (ΔΔC_{T} method). All primers, unless otherwise noted, were designed with Primer Express (Applied Biosystems). qRT-PCR primer sequences are provided in Table 2.

### Drug Treatments

Simvastatin was activated by alkaline hydrolysis to the acidic form prior to usage as previously described (Sadeghi et al., 2000). Briefly, 5 mg of the Simvastatin pro-drug was dissolved in 0.125 ml of 95% ethanol, followed by 0.15 ml of 0.1 N NaOH and the solution was incubated at 50° C. for 2 hr. The final solution was brought to a pH of ˜7.2. Working solutions were stored in DMSO.

Cells were treated on Day 1 or Day 4 of the 3D protocol (as described in the figure legends) and refed every 4 days with fresh drug. The drugs used were:
Simvastatin or Mevastatin at the following concentrations: 100 nM or 1 μM. This range approximates clinically achievable serum concentrations in human patients (Dimitroulakos et al., 1999; Wong et al., 2002),
6-Fluoromevalonate (200 μM) as previously described (Cuthbert and Lipsky, 1990) and YM-53601, FTI-277 or GTI-2133 at or above their reported IC₅₀ in cells (Lerner et al., 1995; Ugawa et al., 2000; Vasudevan et al., 1999), and with
Fatostatin at either 2 μM or 20 μM as previously described (Kamisuki et al., 2009).

### Add-Back Experiments

MDA-468.shp53 or MDA-231.shp53 cells were cultured under 3D conditions in the presence (+DOX) or absence (−DOX) of doxycycline to deplete mutant p53. On Day 1 of 3D culture, cells cultured in the presence of doxycycline were supplemented with DL-Mevalolactone (1 mM)/DL-Mevalonic Acid 5-Phosphate (1 mM) or Geranylgeranyl pyrophosphate (25 μM) and re-fed every 4 days.

MDA-468 or MDA-231 cells were pretreated with DL-Mevalolactone (1 mM)/DL-Mevalonic Acid 5-Phosphate (1 mM) or Geranylgeranyl pyrophosphate (25 μM and then treated with Simvastatin (1 μM).

### Co-Immunoprecipitation of p53

To detect exogenously expressed proteins, sub-confluent HEK 293 cells were transiently transfected with mutant p53 (Flag-p53-R273H) using Lipofectamine 2000 (Invitrogen). Twenty-four hours post-transfection, cells were subjected to formaldehyde crosslinking (1% formaldehyde for 15 min), lysed in RIPA Buffer (150 mM NaCl, 0.1% SDS, 0.5% deoxycholate, 1% NP-40, 5 mM EDTA, 50 mM Tris pH 8.0, 0.5 mM PMSF, protease inhibitors [1 μM benzamidine, 3 μg/ml leupeptin, 0.1 μg/ml bacitracin, and 1 μg/ml macroglobulin]) and sonicated. Anti-Flag antibody (4 μg) with protein A/G Sepharose beads (70 μl 1:1 slurry) were used to immunoprecipitate p53 from 2 mg whole cell lysate. Samples were then subjected to SDS-Page and immunoblotted with anti-Myc or anti-Flag antibodies.

### Quantitative Chromatin Immunoprecipitation

Chromatin Immunoprecipitation (ChIP) experiments were carried out as previously described (Beckerman et al., 2009). Briefly, MDA-468 cells were treated with 1% formaldehyde prior to lysis in RIPA Buffer and sonication to yield 500 bp fragments. Protein A/G Sepharose beads were conjugated to anti-p53 antibodies (1801/DO-1) which were subsequently used to immunoprecipitate p53 from 1 mg whole cell lysate. Quantitative ChIP was carried out on an ABI StepOne Plus using SYBR green dye. Genomic Locations of SRE-1 sites within the promoters of sterol biosynthesis genes were located using a literature search: HMGCS 1 (Inoue et al., 1998), HMGCR (Boone et al., 2009), MVK (Bishop et al., 1998), FDPS (Ishimoto et al., 2010), FDFT1 (Inoue et al., 1998), SQLE (Nagai et al., 2002) and CYP51A1 (Halder et al., 2002), respectively. ChIP primer sequences are provided in Table 2.

### Patient Data

### Hierarchical Clustering

Expression data for the sterol biosynthesis genes were extracted from individual cohorts (FW-MDG, MicMa, Ull, DBCG and Miller). Expression values per gene per dataset were standardized to have mean 0 and standard deviation 1 and further merged across the five datasets.

Unsupervised hierarchical clustering was used to discover groups based on the expression pattern of the sterol biosynthesis genes. In total, 17 sterol biosynthesis genes were used in the unsupervised hierarchical clustering.

Expression values of the 17 sterol biosynthesis genes on 812 samples (where rows indicate the identity of the genes, columns indicate the identity of the patients) were clustered using hierarchical clustering with Euclidean distance and ward linkage. Note that gene MVD was not present in the DBCG dataset; expression values of MVD on 615 samples were used for the distance calculation. The Kaplan-Meier survival curves were plotted for the resulting groups and the differences in clinical indications among the clusters were tested by a logrank test,

### Univariate Survival Analysis for Sterol Biosynthesis Genes

Breast cancer specific death was used as survival endpoint for the analysis (n=533 for MVD and n=723 for others). To remove batch effect across different cohorts, individual gene from each expression dataset was standardized to have mean 0 and standard deviation 1. Expression values per gene per cohort were pulled across the datasets. A univariate Cox proportional hazards model per sterol biosynthesis gene was then fitted:

h(t|X)=h₀(t)exp(β₁X)

where X is the expression vector from the specific gene (variable), β₁ is the coefficient associated with a specific gene, and h₀(t) is the (common) baseline hazard function.
The Hazard Ratio (HR) was used as an accuracy measure for the risk group prediction for categorical predictors. The larger the HR, the better is the discrimination between the groups of the patients, such as low- and high-risk. In our study, continuous covariates entering the Cox models were scaled into mean 0 with standard deviation 1. Thus the estimated HR on the standardized data characterized the relative risk for 1-standard-deviation increase in risk estimation by a specific sterol biosynthesis gene.

Benjamini Hochberg procedure (Benjamini and Hochberg, 1995) was used to adjust multiple comparisons across the tested genes (n=17).

### Expression of Sterol Biosynthesis Gene Versus TP53 Mutation Status

One-tailed t test was performed to assess the significance of the increases in expression level for TP53 mutated samples to those with wild type. The alternative hypothesis Hₐ was expression level of TP53 mutated samples is higher than that of wild type samples.

For individual gene, the test was carried out on five breast cancer datasets: FW-MDG (Haakensen et al., 2010; Muggerud et al., 2010), MicMa (Enerly et al., 2011; Wiedswang et al., 2003), ULL (Langerød et al., 2007), DBCG (Kyndi et al., 2009; Myhre et al., 2010; Nielsen et al., 2006) and Miller (Miller et al., 2005) respectively.

### Combine p Values

Since the datasets do not contain the same patients, the conducted tests in each of the datasets for one gene were independent. An overall significance per gene across datasets was obtained using the Fisher's Omnibus (Fisher, 1932). Benjamini Hochberg procedure (Benjamini and Hochberg, 1995) was used to adjust multiple comparisons across the tested genes. Fisher's method (Fisher, 1932) is used to combine the p values from several independent tests bearing upon the same overall hypothesis (H₀) into one test statistic F:

Fᵢ=−2Σⱼ log(pᵢⱼ)

where pᵢ is the p-value from the hypothesis testing for gene i in the jth dataset (j=1, . . . k; k is the total number of tests being combined; k was five in the study). When all the null hypotheses are true, test statistic F has a chi-square distribution with 2 k degrees of freedom, Therefore, the corresponding overall p-value p₀ᵢ for one gene across all dataset was computed by:

p₀ᵢ=1−χ²(Fᵢ,2k)

### Gene Annotation Mapping

The expression sets were annotated using Entrez gene identities. Genes of interest were mapped to each of the individual sets through Entrez gene IDs. For FW-MDG and MicMa set, the original Agilent probes were mapped to Entrez IDs using BioMart through R library biomaRt (Ensembl release 54/NCBI36 (hg18) human assembly). For Miller set, Affymetrix HG u133a probes were mapped to Entrez IDs by BioMart under the same release. For ULL set, annotations for Stanford 43k cDNA array were retrieved from SMD SOURCE (http://smd.stanford.edu/cgi-bin/source/sourceSearch) under UniGene Build Number 222. Gene identity conversion on DBCG expression set was done using the provided chip annotation file for Applied Biosystem Human Genome Survey Microarray. For the probes shared the same Entrez gene identity, probe(s) with the largest interquartile range (IQR: difference between the third and first quartiles) among the multiple hits were selected. If this still left with more than one hit per Entrez ID, the expression values of those probes for each sample were further averaged.

### Datasets

### FW-MDG

Two expression sets FW (n=109) (Muggerud et al., 2010) and MDG (n=143) (Haakensen et al., 2010) were both from Agilent Whole Human Genome Oligo Microarrays 44k two color system. In addition, they both are early stage breast cancer cohorts and clinically similar. In this study, the two datasets were merged by gene-median centering on the original probe level. Normal samples in the MDG set in the study were also excluded. In total, 139 breast tumors expression profiles with available information on TP53 status entered the analysis. Among these, 28 samples with mutated TP53 status and 111 samples with wild-type status.

### MicMa

This cohort (Wiedswang et al., 2003) consists of mainly stage I and II breast cancers. mRNA expression profiling was performed on Agilent catalogue design whole human genome 4×44K one color oligo array. Among the 112 tumor samples with available TP53 status in this sets, 39 samples with mutated TP53 status and 73 samples with wild-type status.

### ULL

This cohort consists of mainly stage I and II breast cancers. Eighty tumors, along with one normal breast tissue sample, were analyzed using Stanford cDNA 43k two color microarrays. The normal sample in the study was excluded, which left 80 tumor samples for the analysis. Among these, 20 samples with mutated TP53 status and 60 samples with wild-type status.

### DBCG

The DBCG series comprise a collection of tumor tissues from 3,083 high-risk Danish breast cancer patients diagnosed in the period 1982-1990 (Kyndi et al., 2009; Myhre et al., 2010; Nielsen et al., 2006). The profiling was carried out on the Applied Biosystems Human Genome Survey one color Microarray. For this study, there were 46 samples with mutated TP53 status and 104 samples with wild-type status.

### Miller

The Miller dataset (Miller et al., 2005) was downloaded from NCBI's Gene Expression Omnibus (GEO, http://www.ncbi.nlm.nih.gov/geo/) with identifier GSE3494. Data were preprocessed and normalized as described previously (van Vliet et al., 2008). Among the 247 samples, there were 58 samples with mutated TP53 status and 189 samples with wild type status.

### Example 2

Controls were compared to the 3D morphologies of two metastatic breast tumor cell lines that each expresses exclusively a single mutant form of the p53 allele: MDA-231 (R280K) and MDA-468 (R273H). These cells were engineered to stably express a miR30-based doxycycline-inducible shRNA targeting endogenous mutant p53 in the 3′ UTR (designated MDA-231.shp53 and MDA-468.shp53). In both cases mutant p53 reduction by shRNA led to dramatic changes in the behavior of the cells when cultured in a 3D microenvironment. MDA-231 cells, when grown in 3D culture, normally exhibit an extremely disordered and invasive morphology, which has been characterized as “stellate” (Kenny et al., 2007). Depleting these cells of mutant p53 in 3D culture conditions almost completely abrogated the stellate morphology of large, invasive structures with bridging projections (FIG. 1A). Instead, MDA-231 cells with reduced mutant confirm p53 developed smaller, less invasive appearing cell clusters. Depletion of mutant p53 and the accompanying phenotypic effects were highly sensitive to doxycycline-inducible shRNA (FIG. 7A-D). This reduction in invasive behavior in 3D culture supports the recent findings that mutant p53 promotes the invasion of breast cancer cells (Adorno et al., 2009; Muller et al., 2009). Nevertheless, when plated in 3D culture, MDA-231 cells with reduced mutant p53 did not assume the ordered acinus-like morphology that is characteristic of non-malignant mammary epithelial cells.

MDA-468 cells have a less invasive, but highly disorganized appearance, and have been classified as “grape-like” rather than “stellate” (Kenny et al., 2007). Under 3D culture conditions, MDA-468.shp53 cells displayed three types of cellular morphologies (1) constellations of cells with a highly disordered “malignant” appearance that comprise about 30-40% of the population, (2) spherical cell clusters with an “intermediate” morphology that, while disordered, appear less malignant (about 55-65% of the population) and (3) a very small proportion (<5%) of structures that closely resemble small acini and contain a hollow-lumen (examples of these categories are shown in FIG. 1C). Strikingly, when mutant p53 was depleted from these cells, a significant proportion of the population underwent a full phenotypic reversion from highly disorganized structures to acinus-like structures with a hollow-lumen (FIG. 1D). These reverted structures also display proper localization of key integrins, suggesting that they have regained apicobasal polarization (FIG. 7E). Consistent with previous studies implicating programmed cell death in the process of luminal clearance (Debnath et al., 2002), dying cells within the luminal space were occasionally identified (FIG. 1D; image in right panel reveals dying cell within the central luminal region).

There was a significant increase in the hollow-lumen population upon mutant p53 depletion using either a stable pool of MDA-468.shp53 cells (FIG. 1F) or a stable clone derived from these cells (FIG. 1G), with nearly 50% of the MDA-468.shp53 cells showing this acinus-like morphology. In some cases there was a concomitant decrease predominantly in the intermediate population upon the reversion to hollow-lumen structures, and sometimes there was a decrease in both the malignant and intermediate populations. The stable clone of MDA-468.shp53 cells exhibited the highest degree of reversion, therefore all further experiments were carried out using these cells. Importantly, since both of these breast cancer cell lines express only mutant p53, these phenotypic changes may be attributed directly to the reduction in mutant p53 levels.

To confirm and expand upon these observations, MDA-468.shp53 cells were engineered to express an shRNA-resistant version of the p53 mutant that is endogenously found in these cells (p53-R273H) or a control vector (FIG. 2A-B). Introducing excess mutant p53 into these already malignant cells prevented the phenotypic reversion that normally occurs after depleting cells of mutant p53 (FIG. 2B). In fact, exogenous mutant p53, combined with the endogenous level of mutant p53 led to an even more exaggerated malignant phenotype (highly disorganized and invasive) than parental cells (compare left panels of FIGS. 2A and 2B).

Wild-type p53 primarily functions as a transcription factor and the transactivation domains of p53 have previously been implicated in oncogenic functions of mutant p53 such as survival and resistance to chemotherapeutics (Lin et al., 1995; Matas et al., 2001; Yan and Chen, 2010). To interrogate the role of the transactivation domains in the effects of mutant p53 in 3D culture, MDA-468.shp53 cells were engineered to express an shRNA-resistant version of the endogenous mutant p53 that had been mutated at four key residues (L22Q/W23S/W53Q/F54S), shown previously to render its transactivation domains non-functional (Lin et al., 1994; Venot et al., 1999). As opposed to mutant p53 with functional transactivation domains (FIG. 2B), the transactivation-dead version of mutant p53 failed to rescue the phenotypic reversion (FIG. 2C), suggesting that the oncogenic effects in this system were due to transcriptional changes mediated by mutant p53. In order to test whether the effects of mutant p53 on 3D morphology of breast cancer cells were generalizable between tumor-derived mutants of p53, the endogenous mutant p53 in MDA-231 cells (R280K) were replaced with an shRNA-resistant version of p53-R273H, the mutant that is endogenously expressed in MDA-468 cells. While control cells behaved like the cells with just the shRNA-targeting p53, expression of p53-R273H partially prevented the phenotypic changes of knocking down the endogenous p53-R280K (FIG. 7F). The non-malignant human mammary epithelial cell line, MCF10A, was engineered to express Flag-tagged versions of the five most frequent p53 mutants found in breast tumors (p53-R175H, -R248Q, -R273H, -R248W, -G245S) (http://p53.free.fr). MCF10A cells infected with a control vector exhibited normal acinar morphogenesis. However, in agreement with recently published findings (Zhang et al., 2011), expression of the four most frequent mutant p53 proteins led to an inhibition of luminal clearance, reminiscent of the filled lumen phenotype observed in ductal carcinoma in situ (DCIS) lesions (FIG. 8B-F). To examine whether this phenotype was also dependent on the transactivation capacity of p53 mutants, MCF10A cells expressing transactivation-deficient versions (mTAD) of these same five p53 mutants were engineered, which were unable to block luminal clearance (FIG. 81). Thus, not only can depletion of mutant p53 from breast cancer cells lead to a phenotypic reversion in 3D culture, but also mutant p53 expression in non-malignant mammary epithelial cells is sufficient to disrupt their morphology in 3D culture.

### Example 3

### Mutant p53 Upregulates 17 Genes Encoding Enzymes in the Mevalonate Pathway

Since the transactivation activity of mutant p53 is very likely to be critical for its phenotypic effects in 3D culture, genome-wide expression profiling on MDA-468.shp53 cells grown in 3D culture was performed, with or without mutant p53 knockdown. 989 genes were identified as significantly altered (p<0.01) following shRNA-mediated downregulation of endogenous mutant p53, suggesting that mutant p53 acts promiscuously to affect many cellular processes. To guide our identification of those pathways/processes necessary for mutant p53 function in 3D culture, two analysis methods were employed, Ingenuity Pathway Analysis (IPA) and Gene Ontology (GO) Analysis. Since each of these analysis tools has a unique approach for grouping genes according to the pathway or process in which their protein products are reported to function, both methods were exploited in hopes that the pathways/processes that were identified using the two analyses would be more likely to have functional significance. The mevalonate pathway was the most overrepresented cellular pathway using IPA (labeled “Steroid Biosynthesis Pathway” by Ingenuity); in fact, it was the only pathway detected with 99% confidence (p<0.01) following mutant p53 downregulation (FIG. 3A). This pathway, along with the related isoprenoid biosynthetic process, was also detected using GO analysis and was significantly downregulated upon mutant p53 ablation across three independent experiments (FIG. 3B).

Of the many steps that convert Acetyl-CoA to cholesterol, seven genes (HMGCR, MVK, MVD, FDPS, SQLE, LSS, DHCR7) encoding enzymes within the mevalonate pathway were found to be significantly reduced by mutant p53 depletion according to the IPA. In separate experiments, using qRT-PCR expression of all of these genes was confirmed as being markedly reduced (p<0.005) when mutant p53 was depleted by shRNA (FIG. 3C). It was also confirmed that p53-mediated regulation of a subset of these genes in MDA-468 cells occurs as a result of RNA transcription (as opposed to mRNA stability or some later point of regulation) by using primers for nascent transcripts that anneal to intronic regions (data not shown).

The genes that were affected by mutant p53 knockdown encode key enzymes throughout the mevalonate pathway (FIG. 9), including the rate-limiting enzyme, 3-Hydroxy-3-methylglutaryl-CoA reductase (HMG-CoA reductase). The mevalonate pathway is responsible for de novo cholesterol synthesis as well as for many important non-sterol isoprenoid derivatives (FIG. 9) (Goldstein and Brown, 1990).

Elevated or deregulated activity of the mevalonate pathway has been demonstrated in a number of different tumors, including breast cancer (Koyuturk et al., 2007; Wong et al., 2002), and high levels of many of the enzymes in this pathway have been shown to have prognostic significance in breast cancer (Clendening et al., 2010), including breast cancer. This pathway was demonstrated to be necessary for DNA synthesis (Langan and Volpe, 1986; Quesney-Huneeus et al., 1979) and a number of studies have suggested that malignant cells are more highly dependent on the continuous availability of metabolites produced by the mevalonate pathway than their non-malignant counterparts (Buchwald, 1992; Larsson, 1996). While the mevalonate pathway has been explored most extensively in the context of cholesterol production, which is necessary for membrane integrity and thus cell division, many of the intermediate metabolites and final products play key roles in other essential cellular processes. For example, farnesyl pyrophosphate (FPP) and geranylgeranyl pyrophosphate (GGPP) are critical for post-translational modifications of Ras and RhoA, respectively (Casey et al., 1989; Yoshida et al., 1991). Cholesterol is necessary for the formation of steroid hormones, such as estrogen, progesterone and vitamin D (Goldstein and Brown, 1990). Coenzyme Q₁₀ (ubiquinone) plays a vital role in cell respiration, dolichol is essential for N-linked glycosylation of proteins and mevalonic acid has been suggested to promote the cell cycle directly (Graaf et al., 2004; Quesney-Huneeus et al., 1979).

Many of these biologically active intermediate enzymatic products can be readily taken up by cells in culture (Denoyelle et al., 2001). To test whether this pathway is necessary for the phenotypic effects of mutant p53, add-back experiments were performed in which breast cancer cells grown in 3D culture were depleted of mutant p53 and supplemented with intermediate metabolites produced by the mevalonate pathway. Addition of the two earliest metabolites, mevalonic acid (MVA) and mevalonic acid phosphate (MVAP), was sufficient to dramatically inhibit the phenotypic reversion caused by mutant p53 knockdown in MDA-468 cells (FIGS. 4A and 10A). This confirms that activity of the mevalonate pathway is sufficient to compensate for the loss of mutant p53 and suggests that up-regulation of at least the initial steps of the mevalonate pathway is necessary for the effects of mutant p53 on tissue architecture.

HMG-CoA reductase, which catalyzes the formation of mevalonic acid, is the rate limiting step in cholesterol biosynthesis and is famously the target of numerous cholesterol reducing statins (Katz et al., 2005). The use of statins is well established in the clinic to treat patients with hypercholesterolemia and there have been multiple reports demonstrating that statins can exhibit anti-cancer activity; however, their anti-tumorigenic mechanism has not been firmly established (Campbell et al., 2006; Cao et al., 2011; Koyuturk et al., 2007; Shibata et al., 2003).

It was hypothesized that pharmacologic inhibition of the rate-limiting enzyme in the mevalonate pathway might be sufficient to mimic the effects of knocking down mutant p53. Strikingly, treatment of breast cancer cells in 3D culture with Simvastatin, a lipophilic statin, used at clinically achievable concentrations (Wong et al., 2002), resulted in a reduction in growth in both cell lines, in addition to extensive cell death in MDA-468 cells (FIG. 4B) and a significant reduction of the invasive morphology of MDA-231 cells (FIG. 4C). In fact, in MDA-231 cells the morphological changes seen with either statin treatment or mutant p53 knockdown in were virtually the same. The consequence of inhibiting sterol biosynthesis in MDA-468 cells was even more dramatic than mutant p53 downregulation alone (cell death as opposed to formation of structures with a hollow lumen). On the other hand, inhibition of HMG-CoA reductase in wild-type p53 expressing MCF10A cells did not result in gross morphologic changes when used at clinically achievable concentrations (FIG. 10C). This suggests that breast cancer cells bearing mutations in p53 upregulate the mevalonate pathway and eventually become dependent upon its activity for survival. Similar results were obtained with another lipophilic statin, Mevastatin (FIGS. 10D and E). Importantly, supplementation of mevalonic acid, the enzymatic product of HMG-CoA reductase, to either MDA-468 or MDA-231 cells treated with a statin blocked many of the phenotypic effects of statins (Compare FIG. 4D to 4B or 4C). These results indicate that the effects of statins on breast cancer cells in 3D culture occur because of the function of HMG-CoA reductase to produce mevalonic acid, and further implicate the upregulated mevalonate pathway in the malignant 3D phenotype of these cells. An experiment tested whether flux through the mevalonate pathway was sufficient to disrupt normal acinar morphogenesis. MCF10A cells were cultured in 3D culture with or without supplementation of mevalonic acid (MVA) and demonstrate that similar to overexpression of tumor-derived mutants of p53, exogenous mevalonic acid is sufficient to block luminal clearance in MCF10A cells (FIG. 4E).

An experiment was done to test whether statin treatment had an impact on anchorage-independent growth and demonstrate that Simvastatin can significantly impair anchorage-independent growth in both MDA-468 and MDA-231 cells (FIG. 11A). Inhibition of HMG-CoA reductase has previously been reported to induce cell cycle arrest and/or apoptosis in a variety of cell lines grown in traditional (2D) cell culture (Jakobisiak et al., 1991; Sanchez et al., 2008). In line with these findings, it was noted that a G1 cell cycle arrest, with a concomitant drop in S phase, in both breast cancer cell lines treated with 24 hours of Simvastatin at varying concentrations (FIG. 11B-D). The observed phenotypic effects of statins in 3D culture may be due to a combination of factors (i.e. decreased growth, increased death and decreased invasion).

Another experiment tested whether inhibition of later enzymes within the mevalonate pathway would have similar phenotypic effects as mutant p53 depletion from breast cancer cells grown in 3D culture. An inhibitor of Mevalonate Decarboxylase, 6-Fluoromevalonate was applied at a concentration of 200 μM, which was the published dosage in Cuthbert and Lipsky, 1009. 6-fluoromevalonate had remarkably similar phenotypic effects on both MDA-468 and MDA-231 cells grown in 3D culture to that seen with inhibition of HMG-CoA reductase by statins (FIG. 10). Thus, not only HMG-CoA reductase, but several downstream enzymatic steps in the mevalonate pathway are involved in the ability of mutant p53 to prevent normal morphological behavior of breast cancer cells in 3D culture conditions.

Because the mevalonate pathway is not only vital for producing cellular cholesterol but also is important for many other biologically active intermediate metabolites, an experiment examined whether the phenotypic effects of mutant p53 knockdown were due to decreased cholesterol synthesis or the production of an earlier metabolite. To do this, three inhibitors that inhibit distinct actions of the mevalonate pathway were utilized. YM-53601 inhibits squalene synthase (and thus cholesterol production) at submicromolar concentrations (Ugawa et al., 2000), but spares all upstream intermediate metabolites. FTI-277 blocks farnesylation of proteins via inhibition of farnesyltransferase at nanomolar concentrations in whole cells, but has no effect geranylgeranyl transferase or squalene synthesis at low micromolar concentrations (Lerner et al., 1995). GTI-2133 blocks geranylgeranylation of target proteins via inhibition of geranylgeranyl transferase, while sparing farnesylation and squalene synthesis (Vasudevan et al., 1999). See FIG. 9.

While inhibition of squalene synthase (YM-53601, 1 uM) and farnesyl transferase (FTI-277, 1 uM) had only a mild effect on the growth of MDA-231 cells in 3D culture, inhibition of gerananylgeranylation (GTI-2133, 1 uM) had a profound impact on both the growth and the invasive morphology of these cells in 3D culture (FIG. 5A). At higher concentrations of 10 uM the squalene synthase inhibitor YM-53601 killed all the cancer cells (FIG. 5A). To examine whether downregulation of geranylgeranylation is necessary for the phenotypic effects observed after mutant p53 depletion or HMG-CoA Reductase inhibition, add-back experiments were performed adding GGPP to cells either depleted of mutant p53 or cells treated with Simvastatin (FIGS. 5B and C, respectively). Since both were sufficient, these experiments demonstrate that exogenous precursors to prenylation are sufficient to rescue the invasive phenotype of MDA-231 cells in 3D culture, this shows that protein prenylation is a vital component of why breast cancers have selected for mutant p53 upregulation of the mevalonate pathway.

### Example 4

### Patient Data from Five Datasets Shows that a TP53 Mutation Correlates with Elevated Expression of the Mevalonate Pathway Genes

To investigate whether the regulation of the mevalonate pathway by mutant p53 is generalizable to human patients, five datasets were examined consisting of a total of 728 breast cancer patients. Each of these tumor specimens was previously subjected to both genome-wide expression analysis as well as sequencing of TP53 (Enerly et al., 2011; Haakensen et al., 2010; Kyndi et al., 2009; Langerod et al., 2007; Miller et al., 2005; Muggerud et al., 2010; Myhre et al., 2010; Nielsen et al., 2006; Wiedswang et al., 2003). After stratifying patients based on the p53 mutational status of their tumors, the expression level of the sterol biosynthesis genes that had been previously identified as being regulated by mutant p53 in breast cancer cells grown in 3D culture was investigated. Remarkably, eleven of these sterol biosynthesis enzymes exhibited significantly higher expression levels in mutant p53 breast tumors compared to those bearing wild-type p53 across multiple datasets (FIG. 6A and Table 1).

The reciprocal analysis was also performed on these same breast cancer patient datasets, stratifying based on expression of the mevalonate pathway genes and examining the mutation rate of TP53. Three main clusters were observed from the hierarchical clustering of expression matrix from 17 sterol biosynthesis genes on 812 human breast cancer patient samples (728 of these had known TP53 mutational status). Cluster I has the lowest sterol biosynthesis gene expression pattern and the lowest rate of TP53 mutations (46/327=14.1%). Cluster III exhibits an intermediate expression level in these sterol biosynthesis genes and an intermediate rate of TP53 mutations (94/272=34.6%). Cluster II has the highest expression pattern of sterol biosynthesis genes and exhibits the highest rate of TP53 mutations (51/129=39.5%) (FIG. 6B).

To test the biological significance of elevation of the mevalonate pathway in mutant p53 tumors, it was examined whether upregulation of this pathway correlated with patient prognosis. It was demonstrated that cluster I, which has the lowest expression level of the mevalonate pathway genes is correlated with a favorable prognosis, while cluster III, which has an intermediate expression pattern, correlates with an intermediate prognosis and cluster II, which has the highest expression of the mevalonate pathway genes, is associated with a significantly poorer survival probability. Therefore, not only was elevation of the mevalonate pathway significantly correlated to a higher rate of p53 mutations, but these breast cancer patients also had a significantly decreased survival (FIG. 6C). Each sterol biosynthesis gene was then examined individually to investigate which genes contribute most to the prognostic value. Elevated expression of nine out of the seventeen sterol biosynthesis genes correlated with significantly poorer prognosis in these breast cancer patients (FIG. 6D). Since breast cancer cells bearing mutant p53 appear to be particularly sensitive to inhibition of the mevalonate pathway in the 3D culture system, the fact that multiple members of this pathway are upregulated in mutant p53-expressing human tumors and correlate with a poor prognosis has important therapeutic implications.

### Example 5

### In Vivo Data

Simvastatin significantly impacts tumor growth in vivo. 2×10⁶ MDA-231 cells were injected subcutaneously into 8 week-old NOD-SCID mice. Fourteen days after implantation mice were paired by equal tumor volumes and randomized to either a Simvastatin (200 mg/kg/day) or Control (placebo) group (N=5 for each group). Tumor measurements were performed weekly using calipers. After 21 days of treatment, mice were sacrificed and tumors were extracted and weighed. Tumor volumes as a function of time (left) and tumor weights at day 21 (right) are presented. *denotes p<0.01, **denotes p<0.001 using a two-tailed students t-test.

Table 1. Mutant p53 is Correlated with Higher Expression of a Subset of Sterol Biosynthesis Genes in Human Breast Cancer Patient Datasets, (Related to FIG. 6)

TABLE 1

| | | | | | | | False Detection |
|---|---|---|---|---|---|---|---|
| Gene Symbol | FW-MDG | MicMa | ULL | DBCG | Miller | fisher_p | Rate |
| ACAT2 | 0.011748 | 0.347815 | 0.704917 | 0.003674 | 2.25E−08 | 8.18E−09 | 4.08E−08 |
| HMGCS1 | 0.127913 | 0.15145 | 0.007134 | 0.001947 | 2.02E−05 | 1.20E−07 | 4.80E−07 |
| HMGCR | 0.11815 | 0.752179 | 0.837145 | 0.387852 | 0.082508 | 0.286012 | 0.384015 |
| MVK | 0.157677 | 0.100831 | 0.009703 | 0.022681 | 0.453415 | 0.002896 | 0.005265 |
| PMVK | 0.955099 | 0.994949 | 0.696255 | 0.990954 | 0.998388 | 0.99992 | 0.99992 |
| MVD | 0.00392 | 0.000109 | 0.065227 | NA | 0.004984 | 3.10E−07 | 1.03E−06 |
| IDI1 | 0.027657 | 0.028669 | 3.81E−05 | 0.015609 | 3.88E−06 | 1.14E−10 | 7.62E−10 |
| FDPS | 0.492438 | 0.003366 | 0.009796 | 0.057343 | 5.30E−05 | 8.89E−07 | 2.54E−06 |
| FDFT1 | 0.085485 | 0.508364 | 0.385131 | 0.541292 | 0.751081 | 0.451846 | 0.554807 |
| SQLE | 0.198428 | 0.043982 | 0.000874 | 1.97E−05 | 5.97E−09 | 1.23E−13 | 1.23E−12 |
| LBS | 0.291881 | 0.034423 | 0.059208 | 0.804716 | 0.000391 | 0.000258 | 0.000573 |
| CYP51A1 | 0.473917 | 0.444983 | 0.104892 | 0.636533 | 0.009544 | 0.057907 | 0.089087 |
| TM7SF2 | 0.653223 | 0.896114 | 0.8571 | 0.553747 | 0.440326 | 0.937773 | 0.99992 |
| SC4MOL | 0.621928 | 0.095479 | 0.438532 | 0.200135 | 0.001093 | 0.007209 | 0.012015 |
| NSDHL | 0.107219 | 0.12337 | 0.013431 | 0.007662 | 0.001344 | 1.54E−05 | 3.85E−05 |
| DHCR7 | 0.003674 | 0.000334 | 0.000355 | 0.002898 | 1.88E−09 | 5.55E−16 | 1.11E−14 |
| DHCR24 | 0.709416 | 0.625622 | 0.803540 | 0.995518 | 0.993919 | 0.995484 | 0.99992 |

(A) Five human breast cancer patient datasets, FW-MDG, MicMa, ULL, DBCG and Miller were analyzed to determine whether tumors bearing mutant p53 correlated with higher expression of sterol biosynthesis genes. Patients were stratified based on TP53 status (wild-type vs. mutant) and expression levels for sterol biosynthesis genes were analyzed. p-value represents the result of a one-sided t-test for seventeen sterol biosynthesis genes. The right-hand column provides the False Discovery Rate (FDR) for each gene across the five datasets.

TABLE 2
Table 2. Primer Sequences
qRT-PCR

| Gene Symbol | Forward Primer | Reverse Primer |
|---|---|---|
| RPL32 | TTCCTGGTCCACAACGTCAAG | TGTGAGCGATCTCGGCAC |
| ACAT2 | GCGGCGCGGACCAT | CCTGGACAGGAACAGCAGCTA |
| HMGCS1 | GGGCAGGGCATTATTAGGCTAT | TTAGGTTGTCAGCCTCTATGTTGAA |
| HMGCR | GGCCCAGTTGTGCGTCTT | CGAGCCAGGCTTTCACTTCT |
| MVK | TGGACCTCAGCTTACCCAACA | GACTGAAGCCTGGCCACATC |
| PMVK | CCGCGTGTCTCACCCTTT | GACCGTGCCCTCAGCTCAT |
| MVD | TGAACTCCGCGTGCTCATC | CGGTACTGCCTGTCAGCTTCT |
| IDI1 | TTTCCAGGTTGTTTTACGAATACG | TCCTCAAGCTCGGCTGGAT |
| FDPS | CTTCCTATAGCTGCAGCCATGTAC | GCATTGGCGTGCTCCTTCT |
| FDFT1 | TCAGACCAGTCGCAGTTTCG | CTGCGTTGCGCATTTCC |
| SQLE | CGTGCTCCTCTTGGTACCTCAT | CGGTCAAGGCGGAGATTATC |
| LSS | TGCAGAAGGCTCATGAGTTCCT | TCTGGTAGTCGGGAGGGTTATC |
| CYP51A1 | TGCAGCCTGGCTCTTACCA | AGCTCTGTCCCTGCGTCTGA |
| TM7SF2 | GCCACCCTCACCGCTTT | GCTACCTGCGCCTTCATGTAG |
| SC4MOL | GAAAAGCCGGCACCAAGA | TCAAAGAGAGAATCAGCTCAAACTG |
| NSDHL | AGAATCAGGCCAAGAGATGCA | TGTGCTGCCCCAGGAATC |
| DHCR7 | GGCATCCCAGCTCCAACTC | GGGCTCTCTCCAGTTTACAGATGA |
| DHCR24 | CAAGTACGGCCTGTTCCAACA | CGCACAAAGCTGCCATCA |
| INSIG1 | CCCAACACCTGGCATCATC | ACCACCCCAACCGAGAAGAG |
| ACAT1 | GCAGCGAAGAGGCTCAATG | GCAGCGTCAGCAAATGCTACT |
| AACS | ACCCACTGTTCATCATGTTCTCAT | CGGAATGCACCATGCACTT |
| FASN | CGCTCGGCATGGCTATCT | CTCGTTGAAGAACGCATCCA |
| LDLR | AAGCCATTCACTTCCCCAATC | GCCTCACCGTGCATGTTTTA |
| ELOVL6 | CCAGTCAACTCCTCGCACTTT | TGACCGTGTCCGGTATTTCC |
| SCD | CGGGCGGCAGGTTTC | CTGGGACAAGGTGATGAACATG |

ChIP

| Gene Symbol | (Relative to TSS) | Forward Primer | Reverse Primer |
|---|---|---|---|
| HMGCR | (−2100) | GAGGAAGCGGCACATGGA | TGGTATGGACACAAGGTAGAAAGG |
| HMGCR | (−1100) | TTTTCAAGGTCGGGAGTGATG | ACTTTTTCATATGCCACCTCCTTT |
| HMGCR | (−150) | TGGGACTCGAACGGCTATTG | GAACAGGCACCGCACCAT |
| HMGCR | (+1000) | GCAGAGTCGTAGGAAGCATTTGT | TGGGACGCCGAAATCATG |
| HMGCR | (+2500) | GATGAAGGTGGACGATTGAATTC | CCGTTGCCCTGTGATTACG |
| HMGCR | (+4200) | TTGGTCTTTCCCCTAACCCTTT | AACTGCCACTCTAGCAAGAATTCA |
| HMGCS1 | (−115) | GAGGGAAAATCCTAGCGAGTCA | AGTCCGGCTTCTACCAATCAAA |
| MVK | (−80) | CACTCCCAGGGACTTGTTTCC | GCCGACACGGGTTTTCC |
| FDPS | (−260) | CAGCTGCCCAGGAAGATAATG | CCCCGCTGTGGCTTTG |
| FDFT1 | (−140) | CTCCAATGAGCTTCTAGAGTGTTATCA | GGAAGACCCCGGCCAAT |
| SQLE | (−450) | GCGGAATGAATGGAAACGTT | TTGAGGAGAAGCCTGGAGTGA |
| CYP51A1 | (−190) | GCACCCGGGCACACAA | AGGCGATCAATCCCTGAGAA |
| CDKN1A | (+11443) | TCTGTCTCGGCAGCTGACAT | ACCACAAAAGATCAAGGTGAGTGA |
| (“Negative Site”) | | | |

Site-Directed Mutagenesis

| Gene Symbol | Mutant | Forward Primer | Reverse Primer |
|---|---|---|---|
| TP53 | R175H | ACGGAGGTTGTGAGGCACTGCCCCCACCATGAG | GCGCTCATGGTGGGGGCAGTGCCTCACAACCTC |
| | | CGC | CGT |
| TP53 | R273H | AACAGCTTTGAGGTGCATGTTTGTGCCTGTCCTG | CCCAGGACAGGCACAAACATGCACCTCAAAGCT |
| | | GG | GTT |
| TP53 | R248W | ATGGGCGGCATGAACTGGAGGCCCATCCTCACC | GGTGAGGATGGGCCTCCAGTTCATGCCGCCCAT |
| TP53 | R248Q | ATGGGCGGCATGAACCAGAGGCCCATCCTCACC | GGTGAGGATGGGCCTCTGGTTCATGCCGCCCAT |
| TP53 | G245S | AGTTCCTGCATGGGCTCCATGAACCGGAGGCCC | GGGCCTCCGGTTCATGGAGCCCATGCAGGAACT |
| TP53 | L22Q, W23S | CTCTGAGTCAGGAAACATTTTCAGACCAATCGAA | CAGGAAGTAGTTTCGATTGGTCTGAAAATGTTTC |
| | | ACTACTTCCTG | CTGACTCAGAG |
| TP53 | W53Q, F54S | CCCCGGACGATATTGAACAACAGTCCACTGAAGA | GGACCTGGGTCTTCAGTGGACTGTTGTTCAATAT |
| | | CCCAGGTCC | CGTCCGGGG |

TABLE 3

| | List of known proteins, modulation of which |
|---|---|
| | is sufficient to induce a phenotypic reversion in |
| | tumorigenic breast cells grown in 3D culture, |
| Protein | Reference(s) |
| MMP-9 | (Beliveau et al., 2010) |
| ADAM17 (TACE-1) | (Kenny and Bissell, 2007) |
| Fibronectin | (Sandal et al., 2007) |
| Integrin β1 | (Wang et al., 2002; Wang et al., 1998; |
| | Weaver et al., 1997) |
| Integrin β4 | (Dutta and Shaw, 2008; Gabarra et al., 2010; |
| | Weaver et al., 1997) |
| Integrin α2 | (Zutter et al., 1995) |
| Dystroglycan | (Muschler et al., 2002) |
| E-Cadherin | (Fournier et al., 2009; Meiners et al., 1998; Wang |
| | et al., 2002) |
| CEACAM1 | (Huang et al., 1999) |
| ErbB1 (EGFR) | (Beliveau et al., 2010; Itoh et al., 2007; |
| | Muthuswamy et al., 2001; Wang et al., 2002; |
| | Wang et al., 1998) |
| PI3K | (Beliveau et al., 2010; Isakoff et al., 2005; |
| | Liu et al., 2004; Wang et al., 2002) |
| MEK-1/2 (p42/p44 | (Wang et al., 2002; Wang et al., 1998) |
| MAPK) | |
| Rap-1 | (Itoh et al., 2007) |
| HOXD10 | (Carrio et al., 2005) |
| TACC2 (AZU-1) | (Chen et al., 2000) |

See also FIG. 2 in Bissell, M. J., Kenny, P. A., and Radisky, D. C. (2005).

Microenvironmental regulators of tissue structure and function also regulate tumor induction and progression: the role of extracellular matrix and its degrading enzymes. Cold Spring Harb Symp Quant Biol 70, 343-356.

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application (and in the list below) are hereby expressly incorporated by reference as if set forth herein in their entirety, except where terminology is not consistent with the definitions herein. Although specific terms are employed, they are used as in the art unless otherwise indicated.

### REFERENCES

Adorno, M., Cordenonsi, M., Montagner, M., Dupont, S., Wong, C., Hann, B., Solari, A., Bobisse, S., Rondina, M. B., Guzzardo, V., et al. (2009). A Mutant-p53/Smad complex opposes p63 to empower TGFbeta-induced metastasis. Cell 137, 87-98.
Allred, D. C., Mohsin, S. K., and Fuqua, S. A. (2001). Histological and biological evolution of human premalignant breast disease. Endocr Relat Cancer 8, 47-61.
Baigent, C., Keech, A., Kearney, P. M., Blackwell, L., Buck, G., Pollicino, C., Kirby, A., Sourjina, T., Peto, R., Collins, R., et al. (2005). Efficacy and safety of cholesterol-lowering treatment: prospective meta-analysis of data from 90,056 participants in 14 randomised trials of statins. Lancet 366, 1267-1278.
Beckerman, R., Donner, A. J., Mattia, M., Peart, M. J., Manley, J. L., Espinosa, J. M., and Prives, C. (2009). A role for Chk1 in blocking transcriptional elongation of p21 RNA during the S-phase checkpoint. Genes Dev 23, 1364-1377.
Beliveau, A., Mott, J. D., Lo, A., Chen, E. I., Koller, A. A., Yaswen, P., Muschler, J., and Bissell, M. J. (2010). Raf-induced MMP9 disrupts tissue architecture of human breast cells in three-dimensional culture and is necessary for tumor growth in vivo. Genes Dev 24, 2800-2811.
Bengoechea-Alonso, M. T., and Ericsson, J. (2007). SREBP in signal transduction: cholesterol metabolism and beyond. Curr Opin Cell Biol 19, 215-222.
Benjamini, Y., and Hochberg, Y. (1995). Controlling the false discovery rate: a practical and powerful approach to multiple testing. Journal of the Royal Statistical Society Series B (Methodological) 57, 289-300.
Bensaad, K., Tsuruta, A., Selak, M. A., Vidal, M. N., Nakano, K., Bartrons, R., Gottlieb, E., and Vousden, K. H. (2006). TIGAR, a p53-inducible regulator of glycolysis and apoptosis. Cell 126, 107-120.
Bishop, R. W., Chambliss, K. L., Hoffmann, G. F., Tanaka, R. D., and Gibson, K. M. (1998). Characterization of the mevalonate kinase 5′-untranslated region provides evidence for coordinate regulation of cholesterol biosynthesis. Biochem Biophys Res Commun 242, 518-524.
Bissell, M. J., Kenny, P. A., and Radisky, D. C. (2005). Microenvironmental regulators of tissue structure and function also regulate tumor induction and progression: the role of extracellular matrix and its degrading enzymes. Cold Spring Harb Symp Quant Biol 70, 343-356.
Bissell, M. J., Radisky, D. C., Rizki, A., Weaver, V. M., and Petersen, O. W. (2002). The organizing principle: microenvironmental influences in the normal and malignant breast. Differentiation 70, 537-546.
Blais, L., Desgagne, A., and LeLorier, J. (2000). 3-Hydroxy-3-methylglutaryl coenzyme A reductase inhibitors and the risk of cancer: a nested case-control study. Arch Intern Med 160, 2363-2368.
Boone, L. R., Niesen, M. I., Jaroszeski, M., and Ness, G. C. (2009). In vivo identification of promoter elements and transcription factors mediating activation of hepatic HMG-CoA reductase by T3. Biochem Biophys Res Commun 385, 466-471.
Bossi, G., Marampon, F., Maor-Aloni, R., Zani, B., Rotter, V., Oren, M., Strano, S., Blandino, G., and Sacchi, A. (2008). Conditional RNA interference in vivo to study mutant p53 oncogenic gain of function on tumor malignancy. Cell Cycle 7, 1870-1879.
Brekman, A., Singh, K. E., Polotskaia, A., Kundu, N., and Bargonetti, J. (2011). A p53-independent role of Mdm2 in estrogen-mediated activation of breast cancer cell proliferation. Breast Cancer Res 13, R3.
Brosh, R., and Rotter, V. (2009). When mutants gain new powers: news from the mutant p53 field. Nat Rev Cancer 9, 701-713.
Browning, D. R., and Martin, R. M. (2007). Statins and risk of cancer: a systematic review and metaanalysis. Int J Cancer 120, 833-843.
Buchwald, H. (1992). Cholesterol inhibition, cancer, and chemotherapy. Lancet 339, 1154-1156.
Campbell, M. J., Esserman, L. J., Zhou, Y., Shoemaker, M., Lobo, M., Borman, E., Baehner, F., Kumar, A. S., Adduci, K., Marx, C., et al. (2006). Breast cancer growth prevention by statins. Cancer Res 66, 8707-8714.
Cao, Z., Fan-Minogue, H., Bellovin, D. I., Yevtodiyenko, A., Arzeno, J., Yang, Q., Gambhir, S. S., and Felsher, D. W. (2011). MYC Phosphorylation, Activation, and Tumorigenic Potential in Hepatocellular Carcinoma Are Regulated by HMG-CoA Reductase. Cancer Res 71, 2286-2297.
Casey, P. J., Solski, P. A., Der, C. J., and Buss, J. E. (1989). p21ras is modified by a farnesyl isoprenoid. Proc Natl Acad Sci USA 86, 8323-8327.
Cauley, J. A., Zmuda, J. M., Lui, L. Y., Hillier, T. A., Ness, R. B., Stone, K. L., Cummings, S. R., and Bauer, D. C. (2003). Lipid-lowering drug use and breast cancer in older women: a prospective study. J Womens Health (Larchmt) 12, 749-756.
Chicas, A., Molina, P., and Bargonetti, J. (2000). Mutant p53 forms a complex with Spl on HIV-LTR DNA. Biochem Biophys Res Commun 279, 383-390.
Clendening, J. W., Pandyra, A., Boutros, P. C., El Ghamrasni, S., Khosravi, F., Trentin, G. A., Martirosyan, A., Hakem, A., Hakem, R., Jurisica, I., et al. (2010). Dysregulation of the mevalonate pathway promotes transformation. Proc Natl Acad Sci USA 107, 15051-15056.
Cuthbert, J. A., and Lipsky, P. E. (1990). Inhibition by 6-fluoromevalonate demonstrates that mevalonate or one of the mevalonate phosphates is necessary for lymphocyte proliferation. J Biol Chem 265, 18568-18575.
Datta, S., and Osborne, T. F. (2005). Activation domains from both monomers contribute to transcriptional stimulation by sterol regulatory element-binding protein dimers. J Biol Chem 280, 3338-3345.
Davidoff, A. M., Herndon, J. E., 2nd, Glover, N. S., Kerns, B. J., Pence, J. C., Iglehart, J. D., and Marks, J. R. (1991). Relation between p53 overexpression and established prognostic factors in breast cancer. Surgery 110, 259-264.
Debnath, J., Mills, K. R., Collins, N. L., Reginato, M. J., Muthuswamy, S. K., and Brugge, J. S. (2002). The role of apoptosis in creating and maintaining luminal space within normal and oncogene-expressing mammary acini. Cell 111, 29-40.
Debnath, J., Muthuswamy, S. K., and Brugge, J. S. (2003). Morphogenesis and oncogenesis of MCF-10A mammary epithelial acini grown in three-dimensional basement membrane cultures. Methods 30, 256-268.
Denoyelle, C., Vas se, M., Korner, M., Mishal, Z., Ganne, F., Vannier, J. P., Soria, J., and Soria, C. (2001). Cerivastatin, an inhibitor of HMG-CoA reductase, inhibits the signaling pathways involved in the invasiveness and metastatic properties of highly invasive breast cancer cell lines: an in vitro study. Carcinogenesis 22, 1139-1148.
Di Agostino, S., Strano, S., Emiliozzi, V., Zerbini, V., Mottolese, M., Sacchi, A., Blandino, G., and Piaggio, G. (2006). Gain of function of mutant p53: the mutant p53/NF-Y protein complex reveals an aberrant transcriptional mechanism of cell cycle regulation. Cancer Cell 10, 191-202.
Dimitroulakos, J., Nohynek, D., Backway, K. L., Hedley, D. W., Yeger, H., Freedman, M. H., Minden, M. D., and Penn, L. Z. (1999). Increased sensitivity of acute myeloid leukemias to lovastatin-induced apoptosis: A potential therapeutic approach. Blood 93, 1308-1318.
Enerly, E., Steinfeld, I., Kleivi, K., Leivonen, S. K., Aure, M. R., Russnes, H. G., Ronneberg, J. A., Johnsen, H., Navon, R., Rodland, E., et al. (2011). miRNA-mRNA Integrated Analysis Reveals Roles for miRNAs in Primary Breast Tumors. PLoS One 6, e16915.
Fisher, S. R. A. (1932). Statistical methods for research workers (Genesis Publishing Pvt Ltd).
Friedrich, M. J. (2003). Studying cancer in 3 dimensions: 3-D models foster new insights into tumorigenesis. JAMA 290, 1977-1979.
Fuchs, D., Berges, C., Opelz, G., Daniel, V., and Naujokat, C. (2008). HMG-CoA reductase inhibitor simvastatin overcomes bortezomib-induced apoptosis resistance by disrupting a geranylgeranyl pyrophosphate-dependent survival pathway. Biochem Biophys Res Commun 374, 309-314.
Garwood, E. R., Kumar, A. S., Baehner, F. L., Moore, D. H., Au, A., Hylton, N., Flowers, C. I., Garber, J., Lesnikoski, B. A., Hwang, E. S., et al. (2010). Fluvastatin reduces proliferation and increases apoptosis in women with high grade breast cancer. Breast Cancer Res Treat 119, 137-144.
Ghosh-Choudhury, N., Mandal, C. C., and Ghosh Choudhury, G. (2010). Simvastatin induces derepression of PTEN expression via NFkappaB to inhibit breast cancer cell growth. Cell Signal 22, 749-758.
Goldstein, J. L., and Brown, M. S. (1990). Regulation of the mevalonate pathway. Nature 343, 425-430.
Goldstein, J. L., DeBose-Boyd, R. A., and Brown, M. S. (2006). Protein sensors for membrane sterols. Cell 124, 35-46.
Graaf, M. R., Richel, D. J., van Noorden, C. J., and Guchelaar, H. J. (2004). Effects of statins and farnesyltransferase inhibitors on the development and progression of cancer. Cancer Treat Rev 30, 609-641.
Haakensen, V. D., Biong, M., Lingjaerde, O. C., Holmen, M. M., Frantzen, J. O., Chen, Y., Navjord, D., Romundstad, L., Luders, T., Bukholm, I. K., et al. (2010). Expression levels of uridine 5′-diphospho-glucuronosyltransferase genes in breast tissue from healthy women are associated with mammographic density. Breast Cancer Res 12, R65.
Halder, S. K., Fink, M., Waterman, M. R., and Rozman, D. (2002). A cAMP-responsive element binding site is essential for sterol regulation of the human lanosterol 14alpha-demethylase gene (CYP51). Mol Endocrinol 16, 1853-1863.
Han, J. S., Cao, D., Molberg, K. H., Sarode, V. R., Rao, R., Sutton, L. M., and Peng, Y. (2011). Hormone Receptor Status Rather Than HER2 Status Is Significantly Associated With Increased Ki-67 and p53 Expression in Triple-Negative Breast Carcinomas, and High Expression of Ki-67 but Not p53 Is Significantly Associated With Axillary Nodal Metastasis in Triple-Negative and High-Grade Non-Triple-Negative Breast Carcinomas. Am J Clin Pathol 135, 230-237.
Horton, J. D., Goldstein, J. L., and Brown, M. S. (2002). SREBPs: activators of the complete program of cholesterol and fatty acid synthesis in the liver. J Clin Invest 109, 1125-1131.
Hussain, S. P., and Harris, C. C. (1998). Molecular epidemiology of human cancer: contribution of mutation spectra studies of tumor suppressor genes. Cancer Res 58, 4023-4037.
Inoue, J., Sato, R., and Maeda, M. (1998). Multiple DNA elements for sterol regulatory element-binding protein and NF-Y are responsible for sterol-regulated transcription of the genes for human 3-hydroxy-3-methylglutaryl coenzyme A synthase and squalene synthase. J Biochem 123, 1191-1198.
Ishimoto, K., Tachibana, K., Hanano, I., Yamasaki, D., Nakamura, H., Kawai, M., Urano, Y., Tanaka, T., Hamakubo, T., Sakai, J., et al. (2010). Sterol-regulatory-element-binding protein 2 and nuclear factor Y control human farnesyl diphosphate synthase expression and affect cell proliferation in hepatoblastoma cells. Biochem J 429, 347-357.
Jakobisiak, M., Bruno, S., Skierski, J. S., and Darzynkiewicz, Z. (1991). Cell cycle-specific effects of lovastatin. Proc Natl Acad Sci USA 88, 3628-3632.
Kamisuki, S., Mao, Q., Abu-Elheiga, L., Gu, Z., Kugimiya, A., Kwon, Y., Shinohara, T., Kawazoe, Y., Sato, S., Asakura, K., et al. (2009). A small molecule that blocks fat synthesis by inhibiting the activation of SREBP. Chem Biol 16, 882-892.
Katz, M. S., Minsky, B. D., Saltz, L. B., Riedel, E., Chessin, D. B., and Guillem, J. G. (2005). Association of statin use with a pathologic complete response to neoadjuvant chemoradiation for rectal cancer. Int J Radiat Oncol Biol Phys 62, 1363-1370.
Kenny, P. A., Lee, G. Y., Myers, C. A., Neve, R. M., Semeiks, J. R., Spellman, P. T., Lorenz, K., Lee, E. H., Barcellos-Hoff, M. H., Petersen, O. W., et al. (2007). The morphologies of breast cancer cell lines in three-dimensional assays correlate with their profiles of gene expression. Mol Oncol 1, 84-96.
Kidera, Y., Tsubaki, M., Yamazoe, Y., Shoji, K., Nakamura, H., Ogaki, M., Satou, T., Itoh, T., Isozaki, M., Kaneko, J., et al. (2010). Reduction of lung metastasis, cell invasion, and adhesion in mouse melanoma by statin-induced blockade of the Rho/Rho-associated coiled-coil-containing protein kinase pathway. J Exp Clin Cancer Res 29, 127.
Kim, E., Giese, A., and Deppert, W. (2009). Wild-type p53 in cancer cells: when a guardian turns into a blackguard. Biochem Pharmacol 77, 11-20.
Koyuturk, M., Ersoz, M., and Altiok, N. (2007). Simvastatin induces apoptosis in human breast cancer cells: p53 and estrogen receptor independent pathway requiring signalling through JNK. Cancer Lett 250, 220-228.
Kubatka, P., Zihlavnikova, K., Kajo, K., Pec, M., Stollarova, N., Bojkova, B., Kassayova, M., and Orendas, P. (2011). Antineoplastic effects of simvastatin in experimental breast cancer. Klin Onkol 24, 41-45.
Kumar, A. S., Benz, C. C., Shim, V., Minami, C. A., Moore, D. H., and Esserman, L. J. (2008). Estrogen receptor-negative breast cancer is less likely to arise among lipophilic statin users. Cancer Epidemiol Biomarkers Prey 17, 1028-1033.
Kyndi, M., Overgaard, M., Nielsen, H. M., Sorensen, F. B., Knudsen, H., and Overgaard, J. (2009). High local recurrence risk is not associated with large survival reduction after postmastectomy radiotherapy in high-risk breast cancer: a subgroup analysis of DBCG 82 b&c. Radiother Oncol 90, 74-79.
Lang, G. A., Iwakuma, T., Suh, Y. A., Liu, G., Rao, V. A., Parant, J. M., Valentin-Vega, Y. A., Terzian, T., Caldwell, L. C., Strong, L. C., et al. (2004). Gain of function of a p53 hot spot mutation in a mouse model of Li-Fraumeni syndrome. Cell 119, 861-872.
Langan, T. J., and Volpe, J. J. (1986). Obligatory relationship between the sterol biosynthetic pathway and DNA synthesis and cellular proliferation in glial primary cultures. J Neurochem 46, 1283-1291.
Langerød, A., Zhao, H., Borgan, 0., Nesland, J., Bukholm, I., Ikdahl, T., Karesen, R., Borresen-Dale, A., and Jeffrey, S. (2007). TP53 mutation status and gene expression profiles are powerful prognostic markers of breast cancer. Breast Cancer Res 9, R30.
Langerod, A., Zhao, H., Borgan, O., Nesland, J. M., Bukholm, I. R., Ikdahl, T., Karesen, R., Borresen-Dale, A. L., and Jeffrey, S. S. (2007). TP53 mutation status and gene expression profiles are powerful prognostic markers of breast cancer. Breast Cancer Res 9, R30.
Laptenko, O., and Prives, C. (2006). Transcriptional regulation by p53: one protein, many possibilities. Cell Death Differ 13, 951-961.
Larsson, O. (1996). HMG-CoA reductase inhibitors: role in normal and malignant cells. Crit. Rev Oncol Hematol 22, 197-212.
Lerner, E. C., Qian, Y., Blaskovich, M. A., Fossum, R. D., Vogt, A., Sun, J., Cox, A. D., Der, C. J., Hamilton, A. D., and Sebti, S. M. (1995). Ras CAAX peptidomimetic FTI-277 selectively blocks oncogenic Ras signaling by inducing cytoplasmic accumulation of inactive Ras-Raf complexes. J Biol Chem 270, 26802-26806.
Lim, L. Y., Vidnovic, N., Ellisen, L. W., and Leong, C. O. (2009). Mutant p53 mediates survival of breast cancer cells. Br J Cancer 101, 1606-1612.
Lin, J., Chen, J., Elenbaas, B., and Levine, A. J. (1994). Several hydrophobic amino acids in the p53 amino-terminal domain are required for transcriptional activation, binding to mdm-2 and the adenovirus 5 E1B 55-kD protein. Genes Dev 8, 1235-1246.
Lin, J., Teresky, A. K., and Levine, A. J. (1995). Two critical hydrophobic amino acids in the N-terminal domain of the p53 protein are required for the gain of function phenotypes of human p53 mutants. Oncogene 10, 2387-2390.
Martin, K. J., Patrick, D. R., Bissell, M. J., and Fournier, M. V. (2008). Prognostic breast cancer signature identified from 3D culture model accurately predicts clinical outcome across independent datasets. PLoS One 3, e2994.
Matas, D., Sigal, A., Stambolsky, P., Milyaysky, M., Weisz, L., Schwartz, D., Goldfinger, N., and Rotter, V. (2001). Integrity of the N-terminal transcription domain of p53 is required for mutant p53 interference with drug-induced apoptosis. EMBO J 20, 4163-4172.
Miller, L. D., Smeds, J., George, J., Vega, V. B., Vergara, L., Ploner, A., Pawitan, Y., Hall, P., Klaar, S., Liu, E. T., et al. (2005). An expression signature for p53 status in human breast cancer predicts mutation status, transcriptional effects, and patient survival. Proc Natl Acad Sci USA 102, 13550-13555.
Minutolo, F., Asso, V., Bertini, S., Betti, L., Ciriaco, M., Danesi, R., Gervasi, G., Ghilardi, E., Giovanetti, E., Giannaccini, G., et al. (2005). Variously substituted (phosphonoacetamido)oxy analogues of geranylgeranyl diphosphate (GGdP) as GGdP-transferase (GGTase) inhibitors and antiproliferative agents. Med Chem 1, 239-244.
Mirza, A., Wu, Q., Wang, L., McClanahan, T., Bishop, W. R., Gheyas, F., Ding, W., Hutchins, B., Hockenberry, T., Kirschmeier, P., et al. (2003). Global transcriptional program of p53 target genes during the process of apoptosis and cell cycle progression. Oncogene 22, 3645-3654.
Mori, S., Chang, J. T., Andrechek, E. R., Potti, A., and Nevins, J. R. (2009). Utilization of genomic signatures to identify phenotype-specific drugs. PLoS One 4, e6772.
Muggerud, A. A., Hallett, M., Johnsen, H., Kleivi, K., Zhou, W., Tahmasebpoor, S., Amini, R. M., Botling, J., Borresen-Dale, A. L., Sorlie, T., et al. (2010). Molecular diversity in ductal carcinoma in situ (DCIS) and early invasive breast cancer. Mol Oncol 4, 357-368.
Muller, M., Schleithoff, E. S., Stremmel, W., Melino, G., Krammer, P. H., and Schilling, T. (2006). One, two, three—p53, p63, p73 and chemosensitivity. Drug Resist Updat 9, 288-306.
Muller, P. A., Caswell, P. T., Doyle, B., Iwanicki, M. P., Tan, E. H., Karim, S., Lukashchuk, N., Gillespie, D. A., Ludwig, R. L., Gosselin, P., et al. (2009). Mutant p53 drives invasion by promoting integrin recycling. Cell 139, 1327-1341.
Muthuswamy, S. K., Li, D., Lelievre, S., Bissell, M. J., and Brugge, J. S. (2001). ErbB2, but not ErbB 1, reinitiates proliferation and induces luminal repopulation in epithelial acini. Nat Cell Biol 3, 785-792.
Myhre, S., Mohammed, H., Tramm, T., Alsner, J., Finak, G., Park, M., Overgaard, J., Borresen-Dale, A. L., Frigessi, A., and Sorlie, T. (2010). In silico ascription of gene expression differences to tumor and stromal cells in a model to study impact on breast cancer outcome. PLoS One 5, e14002.
Nagai, M., Sakakibara, J., Nakamura, Y., Gejyo, F., and Ono, T. (2002). SREBP-2 and NF-Y are involved in the transcriptional regulation of squalene epoxidase. Biochem Biophys Res Commun 295, 74-80.
Nielsen, H. M., Overgaard, M., Grau, C., Jensen, A. R., and Overgaard, J. (2006). Study of failure pattern among high-risk breast cancer patients with or without postmastectomy radiotherapy in addition to adjuvant systemic therapy: long-term results from the Danish Breast Cancer Cooperative Group DBCG 82 b and c randomized studies. J Clin Oncol 24, 2268-2275.
Olive, K. P., Tuveson, D. A., Ruhe, Z. C., Yin, B., Willis, N. A., Bronson, R. T., Crowley, D., and Jacks, T. (2004). Mutant p53 gain of function in two mouse models of Li-Fraumeni syndrome. Cell 119, 847-860.
Olivier, M., Langerod, A., Carrieri, P., Bergh, J., Klaar, S., Eyfjord, J., Theillet, C., Rodriguez, C., Lidereau, R., Bieche, I., et al. (2006). The clinical value of somatic TP53 gene mutations in 1,794 patients with breast cancer. Clin Cancer Res 12, 1157-1167.
Peart, M. J., and Prives, C. (2006). Mutant p53 gain of function: the NF-Y connection. Cancer Cell 10, 173-174.
Perou, C. M., Sorlie, T., Eisen, M. B., van de Rijn, M., Jeffrey, S. S., Rees, C. A., Pollack, J. R., Ross, D. T., Johnsen, H., Akslen, L. A., et al. (2000). Molecular portraits of human breast tumours. Nature 406, 747-752.
Petersen, O. W., Ronnov-Jessen, L., Howlett, A. R., and Bissell, M. J. (1992). Interaction with basement membrane serves to rapidly distinguish growth and differentiation pattern of normal and malignant human breast epithelial cells. Proc Natl Acad Sci USA 89, 9064-9068.
Petitjean, A., Achatz, M. I., Borresen-Dale, A. L., Hainaut, P., and Olivier, M. (2007). TP53 mutations in human cancers: functional selection and impact on cancer prognosis and outcomes. Oncogene 26, 2157-2165.
Quesney-Huneeus, V., Wiley, M. H., and Siperstein, M. D. (1979). Essential role for mevalonate synthesis in DNA replication. Proc Natl Acad Sci USA 76, 5056-5060.
Reed, B. D., Charos, A. E., Szekely, A. M., Weissman, S. M., and Snyder, M. (2008). Genome-wide occupancy of SREBP1 and its partners NFY and SP1 reveals novel functional roles and combinatorial regulation of distinct classes of genes. PLoS Genet. 4, e1000133.
Riganti, C., Pinto, H., Bolli, E., Belisario, D. C., Calogero, R. A., Bosia, A., and Cavallo, F. (2011). Atorvastatin modulates anti-proliferative and pro-proliferative signals in Her2/neu-positive mammary cancer. Biochem Pharmacol.
Riley, T., Sontag, E., Chen, P., and Levine, A. (2008). Transcriptional control of human p53-regulated genes. Nat Rev Mol Cell Biol 9, 402-412.
Rotter, V. (1983). p53, a transformation-related cellular-encoded protein, can be used as a biochemical marker for the detection of primary mouse tumor cells. Proc Natl Acad Sci USA 80, 2613-2617.
Sadeghi, M. M., Collinge, M., Pardi, R., and Bender, J. R. (2000). Simvastatin modulates cytokine-mediated endothelial cell adhesion molecule induction: involvement of an inhibitory G protein. J Immunol 165, 2712-2718.
Sampath, J., Sun, D., Kidd, V. J., Grenet, J., Gandhi, A., Shapiro, L. H., Wang, Q., Zambetti, G. P., and Schuetz, J. D. (2001). Mutant p53 cooperates with ETS and selectively up-regulates human MDR1 not MRP1. J Biol Chem 276, 39359-39367.
Sanchez, C. A., Rodriguez, E., Varela, E., Zapata, E., Paez, A., Mas so, F. A., Montano, L. F., and Loopez-Marure, R. (2008). Statin-induced inhibition of MCF-7 breast cancer cell proliferation is related to cell cycle arrest and apoptotic and necrotic cell death mediated by an enhanced oxidative stress. Cancer Invest 26, 698-707.
Sato, R. (2010). Sterol metabolism and SREBP activation. Arch Biochem Biophys 501, 177-181.
Shachaf, C. M., Perez, O. D., Youssef, S., Fan, A. C., Elchuri, S., Goldstein, M. J., Shirer, A. E., Sharpe, 0., Chen, J., Mitchell, D. J., et al. (2007). Inhibition of HMGcoA reductase by atorvastatin prevents and reverses MYC-induced lymphomagenesis. Blood 110, 2674-2684.
Shibata, M. A., Ito, Y., Morimoto, J., and Otsuki, Y. (2004). Lovastatin inhibits tumor growth and lung metastasis in mouse mammary carcinoma model: a p53-independent mitochondrial-mediated apoptotic mechanism. Carcinogenesis 25, 1887-1898.
Shibata, M. A., Kavanaugh, C., Shibata, E., Abe, H., Nguyen, P., Otsuki, Y., Trepel, J. B., and Green, J. E. (2003). Comparative effects of lovastatin on mammary and prostate oncogenesis in transgenic mouse models. Carcinogenesis 24, 453-459.
Sorlie, T., Perou, C. M., Tibshirani, R., Aas, T., Geisler, S., Johnsen, H., Hastie, T., Eisen, M. B., van de Rijn, M., Jeffrey, S. S., et al. (2001). Gene expression patterns of breast carcinomas distinguish tumor subclasses with clinical implications. Proc Natl Acad Sci USA 98, 10869-10874.
Soussi, T., and Lozano, G. (2005). p53 mutation heterogeneity in cancer. Biochem Biophys Res Commun 331, 834-842.
Stambolsky, P., Tabach, Y., Fontemaggi, G., Weisz, L., Maor-Aloni, R., Siegfried, Z., Shiff, I., Kogan, I., Shay, M., Kalo, E., et al. (2010). Modulation of the vitamin D3 response by cancer-associated mutant p53. Cancer Cell 17, 273-285.
Stein E A, L. P., Steiner P. (1993). Lovastatin 5-year safety and efficacy study. Lovastatin Study Groups I through IV. Arch Intern Med 153, 1079-1087.
Ugawa, T., Kakuta, H., Moritani, H., Matsuda, K., Ishihara, T., Yamaguchi, M., Naganuma, S., Iizumi, Y., and Shikama, H. (2000). YM-53601, a novel squalene synthase inhibitor, reduces plasma cholesterol and triglyceride levels in several animal species. Br J Pharmacol 131, 63-70.
Unger, T., Mietz, J. A., Scheffner, M., Yee, C. L., and Howley, P. M. (1993). Functional domains of wild-type and mutant p53 proteins involved in transcriptional regulation, transdominant inhibition, and transformation suppression. Mol Cell Biol 13, 5186-5194.
Vallett, S. M., Sanchez, H. B., Rosenfeld, J. M., and Osborne, T. F. (1996). A direct role for sterol regulatory element binding protein in activation of 3-hydroxy-3-methylglutaryl coenzyme A reductase gene. J Biol Chem 271, 12247-12253.
van Vliet, M. H., Reyal, F., Horlings, H. M., van de Vijver, M. J., Reinders, M. J., and Wessels, L. F. (2008). Pooling breast cancer datasets has a synergetic effect on classification performance and improves signature stability. BMC Genomics 9, 375.
Vargo-Gogola, T., and Rosen, J. M. (2007). Modelling breast cancer: one size does not fit all. Nat Rev Cancer 7, 659-672.
Vasudevan, A., Qian, Y., Vogt, A., Blaskovich, M. A., Ohkanda, J., Sebti, S. M., and Hamilton, A. D. (1999). Potent, highly selective, and non-thiol inhibitors of protein geranylgeranyltransferase-I. J Med Chem 42, 1333-1340.
Venot, C., Maratrat, M., Siena, V., Conseiller, E., and Debussche, L. (1999). Definition of a p53 transactivation function-deficient mutant and characterization of two independent p53 transactivation subdomains. Oncogene 18, 2405-2410.
Vogelstein, B., Lane, D., and Levine, A. J. (2000). Surfing the p53 network. Nature 408, 307-310.
Wang, F., Weaver, V. M., Petersen, O. W., Larabell, C. A., Dedhar, S., Briand, P., Lupu, R., and Bissell, M. J. (1998). Reciprocal interactions between beta1-integrin and epidermal growth factor receptor in three-dimensional basement membrane breast cultures: a different perspective in epithelial biology. Proc Natl Acad Sci USA 95, 14821-14826.
Weaver, V. M., Petersen, O. W., Wang, F., Larabell, C. A., Briand, P., Damsky, C., and Bissell, M. J. (1997). Reversion of the malignant phenotype of human breast cells in three-dimensional culture and in vivo by integrin blocking antibodies. J Cell Biol 137, 231-245.
Wejde, J., Blegen, H., and Larsson, O. (1992). Requirement for mevalonate in the control of proliferation of human breast cancer cells. Anticancer Res 12, 317-324.
Wiedswang, G., Borgen, E., Karesen, R., Kvalheim, G., Nesland, J. M., Qvist, H., Schlichting, E., Sauer, T., Janbu, J., Harbitz, T., et al. (2003). Detection of isolated tumor cells in bone marrow is an independent prognostic factor in breast cancer. J Clin Oncol 21, 3469-3478.
Wong, W. W., Dimitroulakos, J., Minden, M. D., and Penn, L. Z. (2002). HMG-CoA reductase inhibitors and the malignant cell: the statin family of drugs as triggers of tumor-specific apoptosis. Leukemia 16, 508-519.
Wrobel, C. N., Debnath, J., Lin, E., Beausoleil, S., Roussel, M. F., and Brugge, J. S. (2004). Autocrine CSF-1R activation promotes Src-dependent disruption of mammary epithelial architecture. J Cell Biol 165, 263-273.
Wu, W. S., Heinrichs, S., Xu, D., Garrison, S. P., Zambetti, G. P., Adams, J. M., and Look, A. T. (2005). Slug antagonizes p53-mediated apoptosis of hematopoietic progenitors by repressing puma. Cell 123, 641-653.
Yahagi, N., Shimano, H., Matsuzaka, T., Najima, Y., Sekiya, M., Nakagawa, Y., Ide, T., Tomita, S., Okazaki, H., Tamura, Y., et al. (2003). p53 Activation in adipocytes of obese mice. J Biol Chem 278, 25395-25400.
Yan, W., and Chen, X. (2010). Characterization of functional domains necessary for mutant p53 gain of function. J Biol Chem 285, 14229-14238.
Yoshida, Y., Kawata, M., Katayama, M., Horiuchi, H., Kita, Y., and Takai, Y. (1991). A geranylgeranyltransferase for rhoA p21 distinct from the farnesyltransferase for ras p21S. Biochem Biophys Res Commun 175, 720-728.
Zhan, L., Rosenberg, A., Bergami, K. C., Yu, M., Xuan, Z., Jaffe, A. B., Allred, C., and Muthuswamy, S. K. (2008). Deregulation of scribble promotes mammary tumorigenesis and reveals a role for cell polarity in carcinoma. Cell 135, 865-878.
Zhang, Y., Yan, W., and Chen, X. (2011). Mutant p53 disrupts MCF-10A cell polarity in 3-dimensional culture via epithelial-to-mesenchymal transitions. J Biol Chem.
Zhuang, L., Lin, J., Lu, M. L., Solomon, K. R., and Freeman, M. R. (2002). Cholesterol-rich lipid rafts mediate akt-regulated survival in prostate cancer cells. Cancer Res 62, 2227-2231.

### REFERENCES FOR TABLE 3

Arias-Romero, L. E., Villamar-Cruz, O., Pacheco, A., Kosoff, R., Huang, M., Muthuswamy, S. K., and Chemoff, J. (2010). A Rac-Pak signaling pathway is essential for ErbB2-mediated transformation of human breast epithelial cancer cells. Oncogene 29, 5839-5849.
Beliveau, A., Mott, J. D., Lo, A., Chen, E. I., Koller, A. A., Yaswen, P., Muschler, J., and Bissell, M. J. (2010). Raf-induced MMP9 disrupts tissue architecture of human breast cells in three-dimensional culture and is necessary for tumor growth in vivo. Genes Dev 24, 2800-2811.
Carrio, M., Arderiu, G., Myers, C., and Boudreau, N. J. (2005). Homeobox D10 induces phenotypic reversion of breast tumor cells in a three-dimensional culture model. Cancer Res 65, 7177-7185.
Chen, H. M., Schmeichel, K. L., Mian, I. S., Lelievre, S., Petersen, O. W., and Bissell, M. J. (2000). AZU-1: a candidate breast tumor suppressor and biomarker for tumor progression. Mol Biol Cell 11, 1357-1367.
Debnath, J., Walker, S. J., and Brugge, J. S. (2003). Akt activation disrupts mammary acinar architecture and enhances proliferation in an mTOR-dependent manner. J Cell Biol 163, 315-326.
Dutta, U., and Shaw, L. M. (2008). A key tyrosine (Y1494) in the beta4 integrin regulates multiple signaling pathways important for tumor development and progression. Cancer Res 68, 8779-8787.
Fournier, M. V., Fata, J. E., Martin, K. J., Yaswen, P., and Bissell, M. J. (2009). Interaction of E-cadherin and PTEN regulates morphogenesis and growth arrest in human mammary epithelial cells. Cancer Res 69, 4545-4552.
Furuta, S., Jiang, X., Gu, B., Cheng, E., Chen, P. L., and Lee, W. H. (2005). Depletion of BRCA1 impairs differentiation but enhances proliferation of mammary epithelial cells. Proc Natl Acad Sci USA 102, 9176-9181.
Gabarra, V., Cho, S., Ramirez, M., Ren, Y., Chen, L. L., Cheung, A., Cao, X., Rennard, R., Unruh, K. R., Graff, C. P., et al. (2010). Antibodies directed to alpha6beta4 highlight the adhesive and signaling functions of the integrin in breast cancer cell lines. Cancer Biol Ther 9, 437-445.
Huang, J., Hardy, J. D., Sun, Y., and Shively, J. E. (1999). Essential role of biliary glycoprotein (CD66a) in morphogenesis of the human mammary epithelial cell line MCF10F. J Cell Sci 112 (Pt 23), 4193-4205.
Irie, H. Y., Pearline, R. V., Grueneberg, D., Hsia, M., Ravichandran, P., Kothari, N., Natesan, S., and Brugge, J. S. (2005). Distinct roles of Akt1 and Akt2 in regulating cell migration and epithelial-mesenchymal transition. J Cell Biol 171, 1023-1034.
Irie, H. Y., Shrestha, Y., Selfors, L. M., Frye, F., Lida, N., Wang, Z., Zou, L., Yao, J., Lu, Y., Epstein, C. B., et al. (2010). PTK6 regulates IGF-1-induced anchorage-independent survival. PLoS One 5, e11729.
Isakoff, S. J., Engelman, J. A., Irie, H. Y., Luo, J., Brachmann, S. M., Pearline, R. V., Cantley, L. C., and Brugge, J. S. (2005). Breast cancer-associated PIK3CA mutations are oncogenic in mammary epithelial cells. Cancer Res 65, 10992-11000.
Itoh, M., Nelson, C. M., Myers, C. A., and Bissell, M. J. (2007). Rapl integrates tissue polarity, lumen formation, and tumorigenic potential in human breast epithelial cells. Cancer Res 67, 4759-4766.
Jung, K. K., Liu, X. W., Chirco, R., Fridman, R., and Kim, H. R. (2006). Identification of CD63 as a tissue inhibitor of metalloproteinase-1 interacting cell surface protein. EMBO J 25, 3934-3942. Kenny, P. A., and Bissell, M. J. (2007). Targeting TACE-dependent EGFR ligand shedding in breast cancer. J Clin Invest 117, 337-345.
Litzenburger, B. C., Kim, H. J., Kuiatse, I., Carboni, J. M., Attar, R. M., Gottardis, M. M., Fairchild, C. R., and Lee, A. V. (2009). BMS-536924 reverses IGF-IR-induced transformation of mammary epithelial cells and causes growth inhibition and polarization of MCF7 cells. Clin Cancer Res 15, 226-237.
Liu, H., Radisky, D. C., Wang, F., and Bissell, M. J. (2004). Polarity and proliferation are controlled by distinct signaling pathways downstream of PI3-kinase in breast epithelial tumor cells. J Cell Biol 164, 603-612.
Liu, X. W., Taube, M. E., Jung, K. K., Dong, Z., Lee, Y. J., Roshy, S., Sloane, B. F., Fridman, R., and Kim, H. R. (2005). Tissue inhibitor of metalloproteinase-1 protects human breast epithelial cells from extrinsic cell death: a potential oncogenic activity of tissue inhibitor of metalloproteinase-1. Cancer Res 65, 898-906.
Liu, Y., Chen, N., Cui, X., Zheng, X., Deng, L., Price, S., Karantza, V., and Minden, A. (2010). The protein kinase Pak4 disrupts mammary acinar architecture and promotes mammary tumorigenesis. Oncogene 29, 5883-5894.
Lochter, A., Galosy, S., Muschler, J., Freedman, N., Werb, Z., and Bissell, M. J. (1997). Matrix metalloproteinase stromelysin-1 triggers a cascade of molecular alterations that leads to stable epithelial-to-mesenchymal conversion and a premalignant phenotype in mammary epithelial cells. J Cell Biol 139, 1861-1872.
Meiners, S., Brinkmann, V., Naundorf, H., and Birchmeier, W. (1998). Role of morphogenetic factors in metastasis of mammary carcinoma cells. Oncogene 16, 9-20.
Muschler, J., Levy, D., Boudreau, R., Henry, M., Campbell, K., and Bissell, M. J. (2002). A role for dystroglycan in epithelial polarization: loss of function in breast tumor cells. Cancer Res 62, 7102-7109.
Muthuswamy, S. K., Li, D., Lelievre, S., Bissell, M. J., and Brugge, J. S. (2001). ErbB2, but not ErbB1, reinitiates proliferation and induces luminal repopulation in epithelial acini. Nat Cell Biol 3, 785-792.
Overholtzer, M., Zhang, J., Smolen, G. A., Muir, B., Li, W., Sgroi, D. C., Deng, C. X., Brugge, J. S., and Haber, D. A. (2006). Transforming properties of YAP, a candidate oncogene on the chromosome 11q22 amplicon. Proc Natl Acad Sci USA 103, 12405-12410.
Reginato, M. J., Mills, K. R., Becker, E. B., Lynch, D. K., Bonni, A., Muthuswamy, S. K., and Brugge, J. S. (2005). Bim regulation of lumen formation in cultured mammary epithelial acini is targeted by oncogenes. Mol Cell Biol 25, 4591-4601.
Sandal, T., Valyi-Nagy, K., Spencer, V. A., Folberg, R., Bissell, M. J., and Maniotis, A. J. (2007). Epigenetic reversion of breast carcinoma phenotype is accompanied by changes in DNA sequestration as measured by Alul restriction enzyme. Am J Pathol 170, 1739-1749.
Sun, T., Aceto, N., Meerbrey, K. L., Kessler, J. D., Zhou, C., Migliaccio, I., Nguyen, D. X., Pavlova, N. N., Botero, M., Huang, J., et al. (2011). Activation of multiple proto-oncogenic tyrosine kinases in breast cancer via loss of the PTPN12 phosphatase. Cell 144, 703-718. Wang, F., Hansen, R. K., Radisky, D., Yoneda, T., Barcellos-Hoff, M. H., Petersen, O. W., Turley, E. A., and Bissell, M. J. (2002). Phenotypic reversion or death of cancer cells by altering signaling pathways in three-dimensional contexts. J Natl Cancer Inst 94, 1494-1503.
Wang, F., Weaver, V. M., Petersen, O. W., Larabell, C. A., Dedhar, S., Briand, P., Lupu, R., and Bissell, M. J. (1998). Reciprocal interactions between beta1-integrin and epidermal growth factor receptor in three-dimensional basement membrane breast cultures: a different perspective in epithelial biology. Proc Natl Acad Sci USA 95, 14821-14826.
Weaver, V. M., Petersen, O. W., Wang, F., Larabell, C. A., Briand, P., Damsky, C., and Bissell, M. J. (1997). Reversion of the malignant phenotype of human breast cells in three-dimensional culture and in vivo by integrin blocking antibodies. J Cell Biol 137, 231-245.
Whyte, J., Thornton, L., McNally, S., McCarthy, S., Lanigan, F., Gallagher, W. M., Stein, T., and Martin, F. (2010). PKCzeta regulates cell polarisation and proliferation restriction during mammary acinus formation. J Cell Sci 123, 3316-3328.
Wrobel, C. N., Debnath, J., Lin, E., Beausoleil, S., Roussel, M. F., and Brugge, J. S. (2004). Autocrine CSF-1R activation promotes Src-dependent disruption of mammary epithelial architecture. J Cell Biol 165, 263-273.
Xian, W., Pappas, L., Pandya, D., Selfors, L. M., Derksen, P. W., de Bruin, M., Gray, N. S., Jonkers, J., Rosen, J. M., and Brugge, J. S. (2009). Fibroblast growth factor receptor 1-transformed mammary epithelial cells are dependent on RSK activity for growth and survival. Cancer Res 69, 2244-2251.
Zhan, L., Rosenberg, A., Bergami, K. C., Yu, M., Xuan, Z., Jaffe, A. B., Allred, C., and Muthuswamy, S. K. (2008). Deregulation of scribble promotes mammary tumorigenesis and reveals a role for cell polarity in carcinoma. Cell 135, 865-878.
Zhang, Y., Yan, W., and Chen, X. (2011). Mutant p53 disrupts MCF-10A cell polarity in 3-dimensional culture via epithelial-to-mesenchymal transitions. J Biol Chem.
Zutter, M. M., Santoro, S. A., Staatz, W. D., and Tsung, Y. L. (1995). Re-expression of the alpha 2 beta 1 integrin abrogates the malignant phenotype of breast carcinoma cells. Proc Natl Acad Sci USA 92, 7411-7415.

### REFERENCES THAT SUPPORT STATINS AS THERAPEUTIC AGENTS IN BREAST CANCER INCLUDE

(http://www.ncbi.nlm.nih.gov/pubmed/21813413); Ghosh-Choudhury et al. 2010
(http://www.ncbi.nlm.nih.gov/pubmed/20060890); Garwood et al. 2010
(http://www.ncbi.nlm.nih.gov/pubmed/19728082); Kang et al. 2009
(http://www.ncbi.nlm.nih.gov/pubmed/19360310); Mori et al. 2009
(http://www.ncbi.nlm.nih.gov/pubmed/19714244); Sanchez et al. 2008
(http://www.ncbi.nlm.nih.gov/pubmed/18608208); Kumar et al. 2008
(http://www.ncbi.nlm.nih.gov/pubmed/18463402); Kwan et al. 2008 Ahern
(http://www.ncbi.nlm.nih.gov/pubmed/17674197); Koyuturk et al. 2007
(http://www.ncbi.nlm.nih.gov/pubmed/17125918); Campbell et al. 2007
(http://www.ncbi.nlm.nih.gov/pubmed/16951186); and Kusama et al. 2006
(http://www.ncbi.nlm.nih.gov/pubmed/16773203).

### REFERENCES THAT DO NOT SUPPORT THE USE OF STATINS TO TREAT BREAST CANCER INCLUDE

Hippisley-Cox et al. 2010 (http://www.ncbi.nlm.nih.gov/pubmed/20488911); Boudreau et al.
2010 (http://www.ncbi.nlm.nih.gov/pubmed/20377474); Cuzick et al. 2011
(http://www.ncbi.nlm.nih.gov/pubmed/21441069); Jacobs et al. 2011
(http://www.ncbi.nlm.nih.gov/pubmed/21343395); Woditschka et al. 2010
(http://www.ncbi.nlm.nih.gov/pubmed/20729289); Eaton et al. 2009
(http://www.ncbi.nlm.nih.gov/pubmed/20044629); Haukka et al. 2010
(http://www.ncbi.nlm.nih.gov/pubmed/19739258); Lubet et al. 2009
(http://www.ncbi.nlm.nih.gov/pubmed/19196723); Kuoppala et al. 2008
(http://www.ncbi.nlm.nih.gov/pubmed/18707867); Taylor et al. 2008
(http://www.ncbi.nlm.nih.gov/pubmed/18414198); Boudreau et al. 2007
(http://www.ncbi.nlm.nih.gov/pubmed/17372235); and Coogan et al. 2007
(http://www.ncbi.nlm.nih.gov/pubmed/17235211).

What is claimed is:

1. A method for determining if a subject having cancer, precancerous cells or a benign tumor will respond to treatment with an inhibitor selected from the group comprising an inhibitor of one or more enzymes in the mevalonate pathway, an inhibitor of geranylgeranyl transferase, or an inhibitor of farnesyl transferase, comprising:
(i) obtaining a sample of the cancer cells, the precancerous cells or the benign tumor cells from the subject,
(ii) assaying the cells in the sample for the presence of a mutated p53 gene or a mutant form of p53 protein or a biologically active fragment thereof, and
(iii) if the cells have the mutated p53 gene or mutant form of the p53 protein, then determining that the subject will respond to treatment with the inhibitor.

2. The method of claim 1, wherein the cancer cells and the precancerous cells are obtained from a tumor or a biological sample from the subject.

3. The method of claim 1, wherein the mutation is detected using an amplification assay, a hybridization assay or by molecular cloning and sequencing or microarray analysis.

4. The method of claim 1, wherein the sample is a tumor biopsy or a biological sample comprising urine, blood, cerebrospinal fluid, sputum, serum, stool or bone marrow.

5. The method of claim 1, in which the p53 gene in the sample is amplified by polymerase chain reaction or a ligase chain reaction.

6. The method of claim 1, in which a DNA hybridization assay is used to detect the p53 gene in the sample.

7. The method of claim 1, wherein the cancer cells are selected from the group comprising lung cancer, digestive and gastrointestinal cancers, gastrointestinal stromal tumors, gastrointestinal carcinoid tumors, colon cancer, rectal cancer, anal cancer, bile duct cancer, small intestine cancer, and stomach (gastric) cancer, esophageal cancer, gall bladder cancer, liver cancer, pancreatic cancer, appendix cancer, breast cancer, ovarian cancer, renal cancer, cancer of the central nervous system, skin cancer, lymphomas, choriocarcinomas, head and neck cancers, osteogenic sarcomas, and blood cancers.

8. The method of claim 1, wherein the cancer is breast cancer that is hormone receptor-negative (ER−/PR−).

9. The method of claim 1, wherein the inhibitor is a statin selected from the group comprising rosuvastatin, lovastatin, simvastatin, pravastatin, rosuvastatin, fluvastatin, atorvastatin, and cerivastatin.

10. The method of claim 1, wherein the inhibitor of geranylgeranyl transferase is GGTI-2133, the inhibitor of farnesyl transferase is selected from the group comprising FTI-277, and the inhibitor of squalene synthase is YM-5360.1.

11. The method of claim 1, wherein the enzyme is HMG-CoA synthase 1, and the inhibitor is 1233A; the enzyme is HMG-CoA reductase and the inhibitor is a statin; the enzyme is mevalonate decarboxylase and the inhibitor is 6-fluormevalonate; the enzyme is isopentyl diphosphate isomerase and the inhibitor is YM-16638; the enzyme is farnesyl diphosphate synthase and the inhibitor is a bisphosphanate that is selected from the group comprising; the enzyme is squalene synthase and the inhibitor is selected from the group comprising YM-53601, qualestatin-1 (zaragozic acid A), RPR-107393, ER-27856, BMS-188494, TAK-475; the enzyme is squalene epoxidase and the inhibitor is TU-2078 or NB-598; the enzyme is anosterol synthase and the inhibitor is Ro 28-8071 fumarate, or BIBB 515; the enzyme is lanosterol 14alpha demethylase and the inhibitor is that is selected from the group comprising SKF 104976, Azalanstat (RS-21607), and Miconazole; the enzyme is cholesterol C4-methyl oxidase and the inhibitor is 3-amino-1,2,4-triazole (ATZ); the enzyme is 7-dehydrocholesterol reductase and the inhibitor is BM 15766 or AY9944; the enzyme is desmosterol reductase and the inhibitor is brassicasterol; the enzyme is farnesyl transferase and the inhibitor is selected from the group comprising Tipifarnib (R115777), Lonafarnib (SCH66336), FTI-277, FTI-276, and FTI-2153; and the enzyme is geranylgeranyl transferase and the inhibitor is selected from the group comprising GGTI-2133, GGTI-2418, GGTI-298, and GGTI-2154.

12. A method for treating a subject having cancer, precancerous cells, or a benign tumor that has a mutated p53 gene or mutant p53 protein, by administering to the subject a therapeutically effective amount of an inhibitor of one or more enzymes in the mevalonate pathway, geranylgeranyl transferase, and farnesyl transferase.

13. The method of claim 12, wherein the cancer is breast cancer that is hormone receptor-negative (ER−/PR−).

14. The method of claim 12, wherein the inhibitor is a statin selected from the group comprising rosuvastatin, lovastatin, simvastatin, pravastatin, rosuvastatin, fluvastatin, atorvastatin, and cerivastatin.

15. The method of claim 12, wherein the inhibitor of geranylgeranyl transferase is GGTI-2133, the inhibitor of farnesyl transferase is selected from the group comprising FTI-277, and the inhibitor of squalene synthase is YM-5360.1.

16. The method of claim 12, wherein the enzyme is HMG-CoA synthase 1, and the inhibitor is 1233A; the enzyme is HMG-CoA reductase and the inhibitor is a statin; the enzyme is mevalonate decarboxylase and the inhibitor is 6-fluormevalonate; the enzyme is isopentyl diphosphate isomerase and the inhibitor is YM-16638; the enzyme is farnesyl diphosphate synthase and the inhibitor is a bisphosphanate that is selected from the group comprising; the enzyme is squalene synthase and the inhibitor is selected from the group comprising YM-53601, qualestatin-1 (zaragozic acid A), RPR-107393, ER-27856, BMS-188494, TAK-475; the enzyme is squalene epoxidase and the inhibitor is TU-2078 or NB-598; the enzyme is anosterol synthase and the inhibitor is Ro 28-8071 fumarate, or BIBB 515; the enzyme is lanosterol 14alpha demethylase and the inhibitor is that is selected from the group comprising SKF 104976, Azalanstat (RS-21607), and Miconazole; the enzyme is cholesterol C4-methyl oxidase and the inhibitor is 3-amino-1,2,4-triazole (ATZ); the enzyme is 7-dehydrocholesterol reductase and the inhibitor is BM 15766 or AY9944; the enzyme is desmosterol reductase and the inhibitor is brassicasterol; the enzyme is farnesyl transferase and the inhibitor is selected from the group comprising Tipifarnib (R115777), Lonafarnib (SCH66336), FTI-277, FTI-276, and FTI-2153; and the enzyme is geranylgeranyl transferase and the inhibitor is selected from the group comprising GGTI-2133, GGTI-2418, GGTI-298, and GGTI-2154.

17. The method of claim 14, wherein the statin is a lipophilic statin selected from the group comprising simvastatin, lovastatin, fluvastatin, cerevastatin and atrovastatin.

18. The method of claim 14, wherein the statin is a hydrophilic statin, selected from the group comprising rosuvastatin and pravastatin.

19. The method of claim 14, wherein the therapeutically effective amount of the statin is from about 0.1 mg/day to about 150 mg/day.

20. The method of claim 12, wherein the inhibitor is administered orally, by injection, parenterally, by inhalation spray, topically, rectally, nasally, buccally, vaginally or via an implanted reservoir.

21. The method of claim 12, wherein the inhibitor is administered locally to the site of the cancer or benign tumor

22. A pharmaceutical formulation comprising one or more statins in a total amount of between 80 mg and 1 gm.

23. The formulation of claim 22, further comprising a member selected from the group comprising a non-statin inhibitor of an enzyme in the mevalonate pathway, an inhibitor of an enzyme in the inhibitor of geranylgeranyl transferase, and an inhibitor of farnesyl transferase.

24. A method for treating cancer, reducing precancerous lesions or benign tumors having a p53 protein or gene mutation in the brain of a subject, comprising administering a therapeutically effective amount of a lipophilic inhibitor selected from the group comprising an inhibitor of one or more enzymes in the mevalonate pathway, an inhibitor of geranylgeranyl transferase, or an inhibitor of farnesyl transferase.

25. The method of claim 24, wherein the inhibitor of an enzyme in the mevalonate pathway is a lipophilic statin selected from the group comprising simvastatin, lovastatin, fluvastatin, cerevastatin and atrovastatin.

26. A method for determining if cancer or precancerous lesions or benign tumors in a mammal will be responsive to treatment with an inhibitor selected from the group comprising an inhibitor of one or more enzymes in the mevalonate pathway, an inhibitor of geranylgeranyl transferase, or an inhibitor of farnesyl transferase, comprising:
(iv) obtaining a sample of the cancer cells, the precancerous cells or the benign tumor cells from the subject,
(v) assaying the cells in the sample for the presence of a mutated p53 gene or a mutant form of p53 protein or a biologically active fragment thereof, and
(vi) if the cells have the mutated p53 gene or mutant form of the p53 protein, then determining that the cancer will respond to treatment with the inhibitor.

27. A method for preventing recurrence of cancer, precancerous lesions or a benign tumor having a mutated p53 gene or a mutant form of p53 protein or a biologically active fragment thereof, comprising administering a prophylactically effective amount of an inhibitor selected from the group comprising an inhibitor of one or more enzymes in the mevalonate pathway, an inhibitor of geranylgeranyl transferase, or an inhibitor of farnesyl transferase.

28. The method of claim 25, wherein the therapeutically effective amount of the statin is from about 80 mg/day to about 1 gram/day.

29. A method of preventing cancer in a subject at high risk of developing comprising a p53 protein or gene mutation, comprising administering an inhibitor selected from the group comprising an inhibitor of one or more enzymes in the mevalonate pathway, an inhibitor of geranylgeranyl transferase, or an inhibitor of farnesyl transferase in a prophylactically effective amount.
